(19) United States
(12) Patent Application Publication
(10) Pub. No.: US 2020/0407712 A1
(43) Pub. Date: Dec. 31, 2020

(54) ARRAY-BASED CYCLIC PEPTIDE LIBRARIES

(71) Applicant: HealthTell Inc., San Ramon, CA (US)

(72) Inventors: James G. BOYD, San Ramon, CA (US); Joseph B. LEGUTKI, San Ramon, CA (US)

(21) Appl. No.: 16/968,512

(22) PCT Filed: Feb. 8, 2019
(86) PCT No.: PCT/US2019/017326

§ 371 Date: Aug. 7, 2020

Related U.S. Application Data
(60) Provisional application No. 62/628,815, filed on Feb. 9, 2018.

(51) Int. Cl. C12N 15/10; G01N 33/68

(52) U.S. Cl. CPC C12N 15/1093; G01N 33/6845; G01N 33/6848

(57) ABSTRACT

Provided herein are in situ generated conformationally constrained peptide arrays, methods for synthesizing such arrays, and methods, systems and assays comprising the use of the synthesized constrained peptide arrays for characterizing protein-target Interactions including: antibody-target interactions, receptor agonist interactions, receptor antagonist interactions, enzyme substrate interactions, enzyme inhibitor interactions, and other protein-protein interactions.

### RELATED APPLICATIONS

This application claims the benefit of U.S. Provisional Patent Application No. 62/628,815 filed on Feb. 9, 2018, which is herein incorporated by reference in its entirety.

### BACKGROUND OF THE INVENTION

Peptide microarrays may be used to detect and characterize peptide-protein or peptide-peptide interactions, including for disease detection and diagnosis. However, the peptide arrays largely include linear peptides on the array surface, which may be too flexible and may not provide sufficient three-dimensional structure required for efficiently detecting protein-peptide or peptide-peptide interactions.

### SUMMARY OF THE INVENTION

Provided herein are conformationally constrained peptide arrays, methods for synthesizing such arrays, and methods, systems and assays comprising the use of in situ synthesized constrained peptide arrays for detecting and characterizing peptide-protein or protein-protein interactions. The compositions, methods and systems disclosed herein may reduce or eliminate non-binding conformational structures, thereby increasing the concentration of binding conformational structures available for binding on the surface of the array. This in turn would lead to an increase in binding of binding molecules to their cognate partner(s) on the surface of the array, increasing sensitivity, specificity or both of arrays incorporating the structurally constrained peptides disclosed herein.

In one aspect, disclosed herein is a peptide array, comprising at least one cyclic peptide feature which comprises peptides of Formula (I):

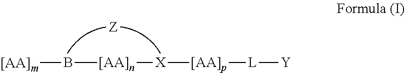

wherein,
each AA is independently a natural or unnatural amino acid residue;
m, n, and p are each independently an integer from 0 to 100;
X is a natural or unnatural amino acid residue;
B is an acid residue; wherein when m is not 0, B is a natural or unnatural amino acid residue;
   wherein each AA, B, and X residue is connected to adjacent residues through peptide bonds;
Z is a linker that connects residues X and B;
L is a tether that is optionally present; and
Y is a point of connection connecting the tether to a solid support having a reactive surface.

In some embodiments, the peptide array is synthesized in situ.

In another aspect, disclosed herein is a method of synthesizing a peptide array, comprising at least one cyclic peptide feature which comprises peptides of Formula (I):

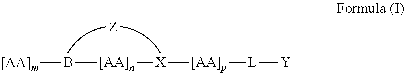

wherein,
   each AA is independently a natural or unnatural amino acid residue;
   m, n, and p are each independently an integer from 0 to 100;
   X is a natural or unnatural amino acid residue;
   B is an acid residue; wherein when m is not 0, B is a natural or unnatural amino acid residue;
      wherein each AA, B, and X residue is connected to adjacent residues through peptide bonds;
   Z is a linker that connects residues X and B;
      wherein Z is

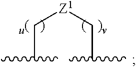

u and v are independently 0-5; and Z¹ is a covalent or non-covalent linkage;
L is a tether that is optionally present; and
Y is a point of connection connecting the tether to a solid support having a reactive surface;
the method comprising the step of:
(a) reacting a functionalized peptide of Formula (II):

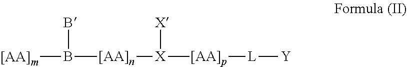

wherein,
   wherein X′ and B′ are complementary groups that combine to form Z¹; or
   X′ and B′ are groups that each combine with a third group Z² to form Z¹;
under conditions that cause linkage Z¹ to form.

In some embodiments, the peptide array is synthesized in situ.

In another aspect, disclosed herein is a method for determining which peptide features of a peptide array have successfully cyclized after a cyclization step, and the % to which they have successfully cyclized, using MALDI mass spectrometry.

In another aspect, disclosed herein is a method for determining which peptide features of a cyclic peptide microarray have successfully cyclized after a cyclization step, and the % to which they have successfully cyclized, the method comprising:
(a) labeling the uncyclized fraction of the cyclic peptide feature with an affinity handle or a reporter probe;
(b) detecting the uncyclized fraction.

In one aspect, disclosed herein is a method for characterizing protein binding to peptide targets, the method comprising:
(a) synthesizing a peptide array comprising at least one cyclic peptide feature which comprises peptides of Formula (I):

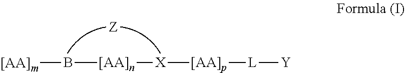

wherein,
each AA is independently a natural or unnatural amino acid residue;
m, n, and p are each independently an integer from 0 to 100;
X is a natural or unnatural amino acid residue;
B is an acid residue; wherein when m is not 0, B is a natural or unnatural amino acid residue;
   wherein each AA, B, and X residue is connected to adjacent residues through peptide bonds;
Z is a linker that connects residues X and B;
L is a tether that is optionally present; and
Y is a point of connection connecting the tether to a solid support having a reactive surface;
(b) contacting said peptide array with said protein at one or more concentrations in the presence and absence of a plurality of competitor molecules at one or more concentrations to obtain one or more individual peptide features, wherein the identified one or more individual peptide features exhibit a binding signal measured in the presence of the plurality of competitor molecules at one or more concentrations within a predetermined threshold of the binding signal measured in the absence of the plurality of competitor peptides; and
(c) characterizing binding of the protein against the peptide features on the peptide array.

In some embodiments, the peptide array is synthesized in situ.

In some embodiments, the method further comprises the steps:
(d) determining which peptide features of said peptide array have successfully cyclized after a cyclization step, and the % to which they have successfully cyclized;
(e) identifying peptide features from the array that have not cyclized to a second predetermined threshold; and
(f) removing peptide features in step (e) from the characterization in step (c).

In another aspect, disclosed herein is a method for characterizing protein binding to peptide targets, the method comprising:
(a) synthesizing a peptide array comprising at least one cyclic peptide feature which comprises peptides of Formula (I):

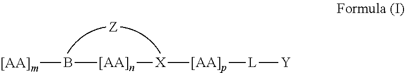

wherein,
each AA is independently a natural or unnatural amino acid residue;
m, n, and p are each independently an integer from 0 to 100;
X is a natural or unnatural amino acid residue;
B is an acid residue; wherein when m is not 0, B is a natural or unnatural amino acid residue;
wherein each AA, B, and X residue is connected to adjacent residues through peptide bonds;
Z is a linker that connects residues X and B;
L is a tether that is optionally present; and
Y is a point of connection connecting the tether to a solid support having a reactive surface;
(b) contacting said peptide array with said protein at one or more concentrations to obtain one or more individual peptide features, wherein the identified one or more individual peptide features exhibit a binding signal measured at one or more concentrations; and
(c) characterizing binding of the protein against the peptide features on the peptide array.

In some embodiments, the peptide array is synthesized in situ.

In some embodiments, the method further comprises the steps:
(d) determining which peptide features of said peptide array have successfully cyclized after a cyclization step, and the % to which they have successfully cyclized;
(e) identifying peptide features from the array that have not cyclized to a predetermined threshold; and
(f) removing peptide features in step (e) from the characterization in step (c).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings in the following.

FIG. 1 illustrates the many low energy conformations available to a linear peptide and how this conformational flexibility dilutes the concentration of complementary conformations available for binding.

FIG. 2 illustrates how conformational constraint eliminates non-complementary conformations and increases the concentration of the protein-bound complex.

FIG. 3 shows examples of conformational constraints: 1) disulfide, 2) thioether, 3) lactam 4) Click triazole, 5) olefin, 6) metal chelation, 7) polynucleic acid intramolecular hybridization, 8) dielectrophile.

FIG. 4 shows one example of how fluorescence labeling of uncyclized peptides can be detected through first labeling with a biotin molecule, followed by detection with a fluorescently labeled streptavidin.

FIG. 5A shows a comparison of yields between Click and head-to-sidechain (H2S) lactam chemistry. Cyclization yields with both chemistries were high for short sequences (X-[AA]ₙ-Z, n<5). H2S lactam chemistry gave consistently high yields for longer sequences (n>5).

FIG. 5B shows comparisons of head-to-sidechain (H2S) and head-to-tail (H2T) cyclization yields for lactam cyclization.

FIG. 6A shows specific fluorescence labeling of uncyclized microarray peptides in the presence of mostly cyclized peptides by first labeling with a biotin molecule, followed by detection with a fluorescently labeled streptavidin.

FIG. 6B demonstrates that biotin labeled intensities from an example peptide, BEPANPSKNSTX, correspond to MALDI data. MALDI determined that cyclization efficiency of Click was 25%, while lactam was 95%.

FIG. 7 shows biotin labeling intensities grouped by MALDI efficiency for both the Click and lactam cyclization. Peptides were binned into the following categories: Click chemistry and lactam both greater than 97% cyclic, Click less than 40% cyclic with lactam greater than 97% cyclic, and Click less than 30% cyclic and lactam between 60 and 95% cyclic.

FIG. 8 shows fluorescence intensity distributions for both the Click and lactam arrays. Linear features lack one of the cyclization residues and are thus solely acyclic. Cyclic features contain both required cyclization residues.

FIG. 9 illustrates the extracellular domain of CD20 which Rituximab targets. Rituximab is known to interact with the amino acid loop EPANPSEKNSPSTQY which is bounded on either side by cysteine residues that form a constrained disulfide bridge.

FIG. 10 shows Rituximab binding to cyclic and linear in situ synthesized anchored peptides of the sequence BAEANPSX in both the Click and lactam cyclized arrays.

FIG. 11 shows cyclization efficiency of both the Click and lactam features used for Rituximab binding assays. Biotinylation data indicates peptide YNBAEANPSX has improved cyclization in lactam chemistry.

FIG. 12 shows the MALDI Mass spectra of the Click cyclization product from Example 5, as determined in Example 7. Top spectrum: DBCO-acid does not react with peptide indicating no azidopeptide remaining following Cu(I) cyclization. (Calculated monoisotopic m/z for cyclized peptide=1574.8, found 1574.4). Bottom spectrum: DBCO-acid reacts quantitatively with azidopeptide that did not undergo Cu(I) cyclization (Calculated monoisotopic m/z for cyclized peptide=1879.8, found 1879.4).

FIG. 13 shows the MALDI Mass spectra of head-to-tail lactam cyclization product from Example 13. Calculated monoisotopic m/z for cyclized peptide=1684.8, found 1684.7). Dark circles indicate peaks from external MW standards added to the matrix. Inset shows relative peak intensities of cyclized lactam product (84%) and linear form (16%).

FIG. 14 shows the MALDI Mass spectra of head-to-sidechain lactam cyclization product from Example 14. Calculated monoisotopic m/z for cyclized peptide=1684.8, found 1684.7). Dark circles indicate peaks from external MW standards added to the matrix. Inset shows relative peak intensities of cyclized lactam product (97%) and linear form (3%).

FIG. 15 shows monoclonal 2H7 binding to lactam constrained peptide arrays from Example 23.

FIG. 16 shows monoclonal 2H7 binding to Click constrained peptide arrays from Example 23.

FIG. 17 shows the Silicon Oxide surface prepared for peptide array synthesis as prepared in Example 1.

FIG. 18 shows the electrospray mass spectrum of cyclo-(KϵAEANPSE)-PEG₃-biotin (HTP115) as synthesized in Example 24.

FIG. 19 shows Rituximab binding to biotinyl peptides captured on streptavidin plates for both cyclic and linear peptides. Assays were run in triplicate.

FIG. 20 shows the synthetic scheme for the synthesis of disulfide constrained peptides on SiO₂ wafers with thallium (III) oxidation as performed in Example 26.

FIG. 21 shows the MALDI mass spectra of: (top) the thiol-protected precursor peptide; and (bottom) sidechain-to-sidechain disulfide cyclization product from Example 26.

FIG. 22 shows the synthetic scheme for the synthesis of disulfide constrained peptides on SiO₂ wafers with air oxidation as performed in Example 27.

FIG. 23 shows MALDI mass spectra of selected disulfide constrained microarray peptides from Example 27. The inset shows expanded spectrum showing both oxidized (cyclic) and reduced (linear) forms of the peptide.

FIG. 24 shows MALDI mass spectra of diaphorase labeled with Alexa Fluor™ 555-NHS (top) and Alexa Fluor™ 647-NHS (bottom).

FIG. 25 shows enzymatic activity of Alexa Fluor™-labeled-diaphorase samples.

FIG. 26A shows the per peptide intensities of the TCEP reduced disulfide arrays as a control for a completely linear array in Example 29 for peptide features with 0, 2, 4, 6, 8, or 10 residues between the cysteine residues. The dashed line is the detector saturation.

FIG. 26B shows the per peptide intensities of disulfide arrays following oxidation in Example 29 for peptide features with 0, 2, 4, 6, 8, or 10 residues between the cysteine residues. The dashed line is the detector saturation.

FIG. 26C shows the delta value of cyclic oxidized disulfide vs linear TCEP reduced arrays to determine the degree of cyclization in Example 29 for peptide features with 0, 2, 4, 6, 8, or 10 residues between the cysteine residues. Delta is calculated as the per peptide difference in log 10 transformed intensities (LFG) between TCEP reduced and oxidized disulfides.

FIG. 27A shows Alexa Fluor™ 555-diaphorase binding to linear and lactam constrained microarrays in Example 31. Intensities are plotted as the log 10 transformed median intensity from replicate arrays.

FIG. 27B shows CLUSTAL sequence alignment of the sequences that exhibit the greatest enhanced affinity in the constrained arrays relative to the linear arrays.

FIG. 28 shows substitution analysis of lactam constrained diaphorase binding peptides, indicating which residues are important for binding, as determined in Example 31.

FIG. 29A shows Alexa Fluor™ 555-diaphorase binding to lactam constrained microarrays and Non-Immunoglobulin Serum Components (NISC) as a counter-screen in Example 31. Intensities are plotted as the log 10 transformed median intensity from replicate arrays.

FIG. 29B shows CLUSTAL sequence alignment of the sequences that exhibit the greatest enhanced affinity in the constrained arrays relative to the NISC counter-screen.

FIG. 30 shows substitution analysis of lactam constrained diaphorase binding peptides that show selectivity over NISC, as determined in Example 31.

### DETAILED DESCRIPTION OF THE INVENTION

Many screens based on peptide arrays suffer from the fact that peptide libraries largely comprise linear peptides. Linear peptides may not present binding structures recognized by a binding partner, in part due to the multiple degrees of conformational freedom both in their backbone and their side chains. Under normal conditions, for example in aqueous solution and at ambient temperature, a peptide molecule rapidly interconverts between the many low energy conformations available to it. Amongst the population of many and varied conformations is/are one(s) complementary to and suitable for a binding to a partner molecule, usually a polypeptide, a protein, a receptor, or an antibody. In order for a population of rapidly interconverting peptide conformers to adopt a single conformation, or a small subset of the allowable conformations, there may be an unfavorable change entropy. This entropy change (ΔS) results in an unfavorable change in the free energy of binding (ΔG) which is related to the observed equilibrium binding constant by the free energy of binding equation (ΔG=ΔH−TΔS=RT ln nK, where R is the ideal gas constant, T is the temperature in ° K, and K is the equilibrium binding constant). Thus linear peptides with many available conformations are predisposed to weaker binding interactions than well-structured peptides with fewer allowed conformations.

Additionally, there are many peptide-protein binding interactions for which little structural information exists, for example, membrane bound proteins such as GPCRs. In these examples a specific peptide design approach is likely to be slow and ineffective and a more effective approach to discover new peptide ligands is also through the use of peptide libraries and peptide arrays. With this approach standard peptide synthesis techniques are used to synthesize simultaneously large numbers of unique peptide sequences (up to 10⁷) on a solid support surface and in a fashion such that the identity of each peptide at each location is known. Following a post-synthesis deprotection step, peptides on an array surface can be tested for complementarity in an assay.

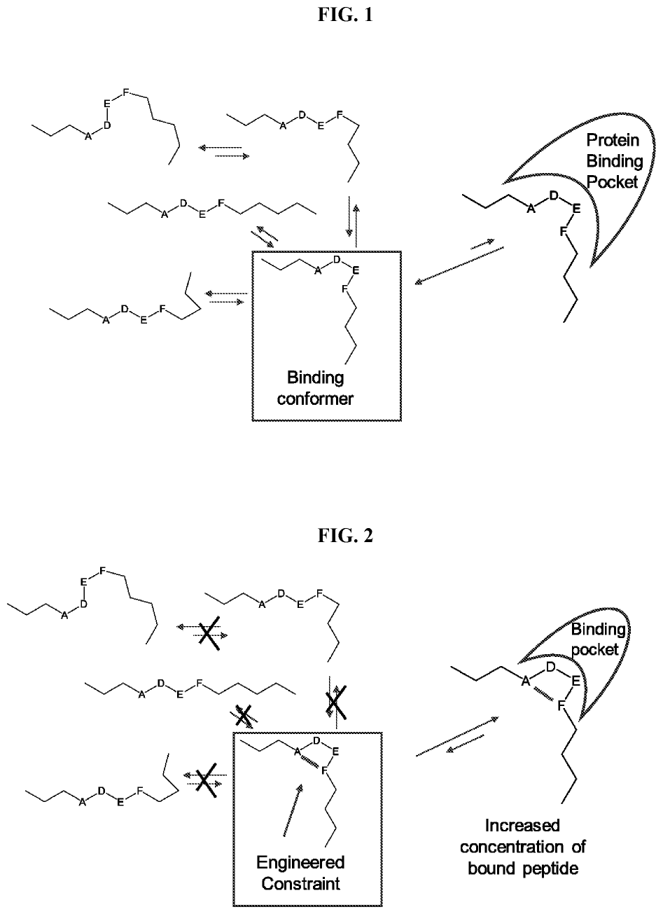

There are many biological screening and diagnostic processes that rely on peptide-protein interactions, for example, antibody-target interactions, receptor agonist interactions, receptor antagonist interactions, enzyme substrate interactions, enzyme inhibitor interactions, and other protein-protein interactions. These interactions can likewise be screened using a peptide array. However, many of these screens may suffer from the fact that the peptide libraries are linear peptides. Linear peptides are floppy structures that have multiple degrees of conformational freedom both in their backbone and their side chains. Under normal conditions, for example in aqueous solution and at ambient temperature, a peptide molecule is rapidly interconverting between the many low energy conformations available to it (FIG. 1). Considering just the backbone atoms, a medium sized peptide of 10 amino acid residues has many thousands of energetically accessible conformations, and at any particular instant individual peptide molecules are sampling each of these. Amongst the population of many and varied conformations is/are one(s) complementary to and suitable for a binding to a partner molecule, usually a protein, protein receptor, or an antibody. In order for a population of peptide molecules to adopt a single conformation, or a small subset of the allowable conformations there is an unfavorable change entropy. This entropy change (ΔS) results in an unfavorable change in the free energy of binding (ΔG) which is related to the observed equilibrium binding constant by the free energy of binding equation (ΔG=ΔH−TΔS=RT ln K, where K is the equilibrium binding constant). Thus floppy peptides with many available conformations are predisposed to weaker binding interactions than well-structured peptides with fewer allowed conformations.

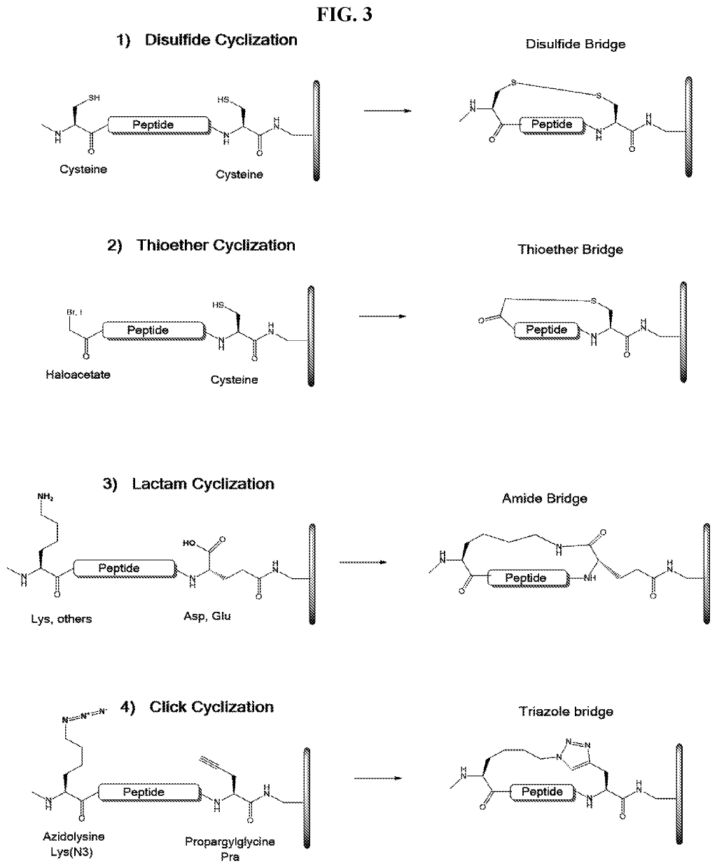

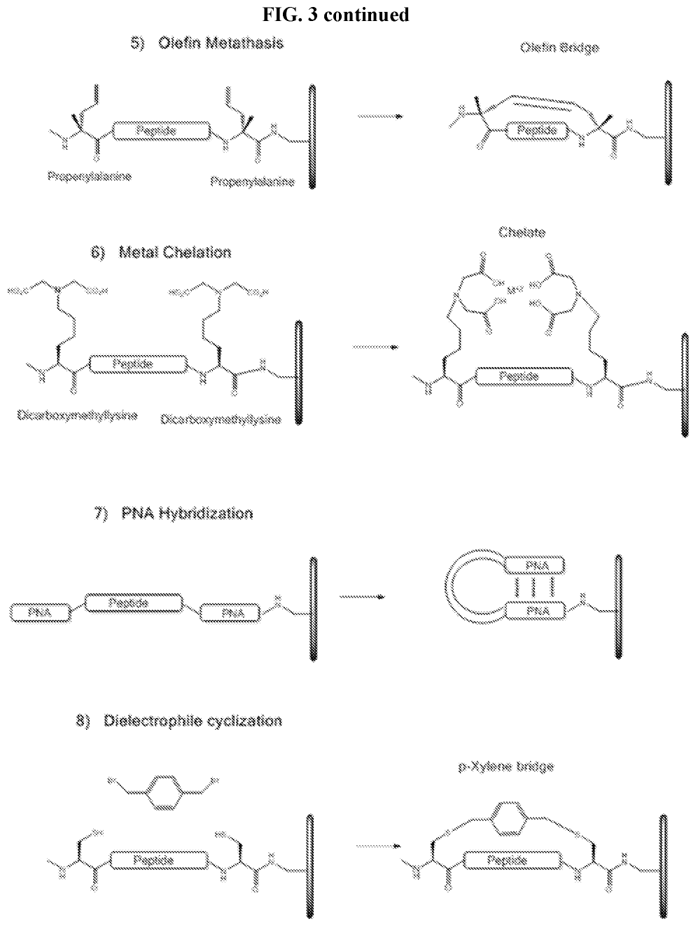

One way to resolve this problem is reduce or eliminate some or all of the nonproductive conformations by making structural changes to the peptide, including the engineering of a covalent bond or several covalent bonds that freeze out degrees of freedom and thus reduce the entropy penalty that is paid during the binding event (FIG. 2). An additional pathway to reduce the degrees of conformational freedom is through a covalent bridge, often a disulfide between two cysteine residues within the peptide. Other options include thioethers, ethers, triazole (Click), and amide (lactam) bridges between natural amino acids or non-natural amino acids and other molecules (FIG. 3). Disulfides have been used extensively due to their ease of use and compatibility with biological systems such as phage libraries. Other covalent bridges, for example amide bridges, are more stable and unlikely to change once formed. Another pathway to reduce the degrees of conformational freedom is through a non-covalent interaction, such as metal chelation, a salt bridge, or hydrogen bonding.

The methods disclosed herein with regards to covalent constriction have limits to their utility and dependability. In order for the constraining bridge to form, the functional groups that form the bridge need to come within a reaction distance and trajectory in order for the bond to form. Factors that can hinder this process include length (number of atoms between the two reactive sites), number of rotatable bonds between the species, number of rigid bonds, temperature, solvent, and others. Since the peptides on an array are diverse structures, one sequence will be predisposed to coming into a productive conformation that promotes the subsequent reaction while another will resist adoption of productive conformations. Thus for a particular peptide with a particular constraining chemistry and particular reaction conditions a particular yield would be observed. But under the exact same conditions, with a change in peptide sequence, or length, or both, a different yield can and often will be observed.

Typical yields for cyclization reaction can range between 0-100% and seemingly similar peptide sequences can give drastically different yields. A yield of between 0-100% means that a mixture of linear and constrained products will reside at that peptide feature. Either linear or constrained peptides could be complimentary, or non-complimentary, to a protein of interest, so it is also desirable to know whether 0% constrained, 100% constrained, or a mixture of constrained and unconstrained is present. A challenge with peptide arrays is the vast number of peptides involved (up to 10⁷, or greater) makes analysis of each and every feature a cumbersome process, so an enabling technology that provides information on the percentage of constrained structures within each and every feature within an array is useful.

The technologies disclosed herein will enable reliable, high-throughput, low-cost and comprehensive binding characterization of peptide-protein interactions utilizing a more biologically relevant array of constrained peptides, rather than an array of linear peptides. The technologies disclosed herein include a highly scalable array-based conformationally constrained peptide library platform based on in situ peptide synthesis. The methods and assays disclosed herein also provide the ability to identify antibody binding regions, including epitopes and putative epitopes, as well as protein targets to antibodies, allowing elucidation of possible off-target proteins that could play a role in, for example, adverse or non-target interactions. In addition, constrained peptides that are useful for mapping antibody epitopes are likely to present that epitope in a conformation that well represents the epitope's native conformation as found within the antibody's actual protein target. The constrained peptides when coupled to available carrier and adjuvant technologies are likely to be effective immunogens for generation of an immune response, for example a polyclonal antibody that recognizes the original protein target. Thus constrained peptide arrays are a useful source of peptide leads that can be developed into peptide-based vaccines. The methods and assays disclosed herein also provide the ability to identify functional peptide leads that can be developed into peptide therapeutic agents for example, peptide agonist or antagonist ligands for protein receptors. Also disclosed herein are assays to determine the extent of beneficial cyclization events contained in such arrays, in order to allow more rigorous analysis of the data to identify, characterize, and/or categorize such binding events.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The terms below, as used herein, have the following meanings, unless indicated otherwise:

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. Unless stated otherwise specifically in the specification, an alkyl group may be optionally substituted as described below.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein an sp2-hybridized carbon of the alkenyl residue is attached to the rest of the molecule by a single bond. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-c₅ alkenyl, a C₂-C₄ alkenyl, a C₂-C₃ alkenyl, or a C₂ alkenyl. Unless stated otherwise specifically in the specification, an alkenyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

The term “aromatic” refers to a planar ring having a delocalized π-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Cycloalkyl” refers to a monocyclic or polycyclic non-aromatic radical, wherein each of the atoms forming the ring (i.e., skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycicoalkyl radicals include, for example, cyclopropyl, cyclobutyl, cyclopentyl, cyclohexyl, cycloheptyl, and cyclooctyl. In some embodiments, the monocyclic cycicoalkyl is cyclopropyl, cyclobutyl, cyclopentyl or cyclohexyl. In some embodiments, the monocyclic cycicoalkyl is cyclopentyl. Polycyclic radicals include, for example, adamantyl, norbornyl, decalinyl, and 3,4-dihydronaphthalen-1(2H)-one. Unless otherwise stated specifically in the specification, a cycloalkyl group may be optionally substituted.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heteroalkyl” refers to an alkyl radical as described above where one or more carbon atoms of the alkyl is replaced with a O, N (i.e., NH, N-alkyl) or S atom. “Heteroalkylene” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g., —NH₂, —NHR, —NR₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

“Amino acid” refers to the class of organic compounds that contain at least one amino group (typically —NH₂, or —NH(alkyl)) and one carboxyl group (—COOH). Amino acids are most typically alpha-amino acids, which contain one optionally substituted carbon atom, the alpha-carbon, between the amino and carboxyl groups, e.g. H₂N—CH(R)—COOH. The optional substitution (R) is referred to as the “side chain.” Amino acids can contain more than one carbon between the amino and carboxyl groups, e.g. 2 carbon atoms (beta-amino acids), 3 carbon atoms (gamma-amino acids), and the like. “Natural amino acids” refer to those commonly used in building naturally occurring proteins, and are the L-isomer, alpha-amino acids. These amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. “Unnatural amino acids” include those that are D-isomers of the natural amino acids, beta-amino acids and the like, and those with sidechains not found in the natural amino acids. Many unnatural amino acids are known and used in the art and may include functionality on their side chains such as alkynes, azides, haloacetates, acetates, aldehydes, thiols, amines, carboxyls, haloalkanes, alkenes, or the like. All natural and unnatural amino acids are contemplated in the present disclosure.

“Peptide” refers to a molecule produced by covalently coupled chain of amino acids generally coupled between the backbone amine and backbone carboxyl groups, but may be coupled between sidechain amine and carboxyl groups. A peptide may be comprised of natural amino acids, comprised of unnatural amino acids or comprised of both natural and unnatural amino acids.

The terms “cyclic” “constrained” “conformationally constrained,” “constrained cyclic,” and the like, as used herein refer to peptides, or peptide features of an array, in which two amino acid residues are attached to each other by covalent bond(s) or non-covalent interactions, other than through the peptide backbone.

The terms “linear” and the like, as used herein refer to peptides, or peptide features of an array, in which amino acids are connected only through the peptide backbone. That is, they are not cyclic, nor conformationally constrained.

### Array Platform and Synthesis

Disclosed herein are methods and processes that provide for array platforms that allow for increased diversity and fidelity of chemical library synthesis. The array platforms comprise a plurality of individual features on the surface of the array. Each feature typically comprises a plurality of individual molecules synthesized in situ on the surface of the array, wherein the molecules are identical within a feature, but the sequence or identity of the molecules differ between features on the array. The array molecules include, but are not limited to, peptides, peptide-mimetics, and combinations thereof and the like, wherein the array molecules may comprise natural or non-natural monomers within the molecules.

Arrays disclosed herein are synthesized using in situ synthesis of peptide arrays on reactive silicon oxide wafers, as disclosed in U.S. Provisional Pat. App. No. 62/317,353, filed Apr. 1, 2016, U.S. Provisional Pat. App. No. 62/472,504, filed Mar. 16, 2017, and PCT Pat. App. No. PCT/US17/25546, filed Mar. 31, 2017, which are hereby incorporated by reference for such purposes. Briefly, the technologies are based on merged peptide synthesis chemistry with semiconductor manufacturing processes by utilizing mask-based photolithography to pattern, in situ, libraries containing up to 10 million or more peptides on an eight-inch wafer. This wafer is diced into 13 microscope-slide dimensioned chips for downstream analysis. With such a peptide library chips, protein-peptide binding profile assays can be scaled to more than 10 million interactions per day at a fraction of the cost of current characterization platforms utilizing constrained peptide arrays.

In one aspect, disclosed herein is a peptide array, comprising at least one cyclic peptide feature which comprises peptides of Formula (I):

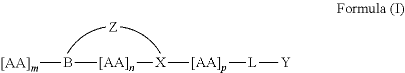

wherein,
each AA is independently a natural or unnatural amino acid residue;
m, n, and p are each independently an integer from 0 to 100;
X is a natural or unnatural amino acid residue;
   B is an acid residue; wherein when m is not 0, B is a natural or unnatural amino acid residue;
wherein each AA, B, and X residue is connected to adjacent residues through peptide bonds;
Z is a linker that connects residues X and B;
L is a tether that is optionally present; and
Y is a point of connection connecting the tether to a solid support having a reactive surface.

In some embodiments, each peptide of Formula (I) is independently represented by Formula (Ia) or Formula (Ib):

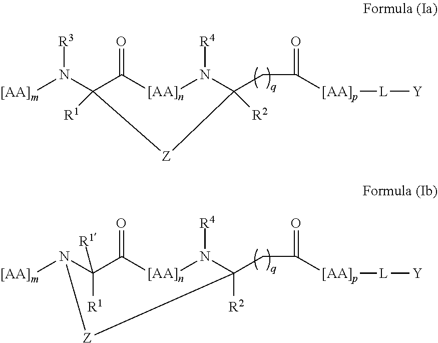

wherein:
   q is 0-5;
   R¹ and R² are each independently hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl;
   R^{1′} is a natural or unnatural amino acid sidechain;
   R³ and R⁴ are each independently hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl.

In some embodiments, q is 0 to 5. In some embodiments, q is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, q is 0, 1, 2, 3, 4, or 5.

In some embodiments, Z of the at least one cyclic peptide feature is

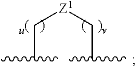

wherein u and v are independently 0-5; and Z¹ is a covalent or non-covalent linkage.

In some embodiments, u is 0 to 5. In some embodiments, u is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, u is 0, 1, 2, 3, 4, or 5.

In some embodiments, v is 0 to 5. In some embodiments, v is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, v is 0, 1, 2, 3, 4, or 5.

It is the intention of this disclosure for each Z to be connecting B and X in either direction. In some embodiments, Z as pictured connects to B and X from left-to-right. In some embodiments, Z as pictured connects to B and X from right-to-left.

In some embodiments, linker Z which connects residues X and B, comprises Z¹; wherein Z¹ is a covalent linkage. In some embodiments, Z¹ is:

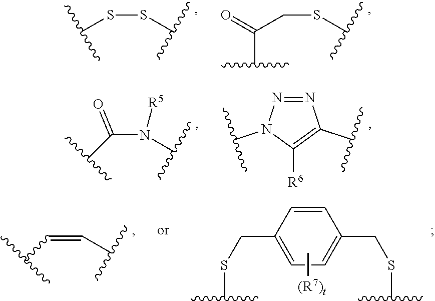

wherein:
   R⁵ is hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl;
   R⁶ and R⁷ are each independently halogen, hydroxy, C₁-C₆ alkyl, C₁-C₆ alkoxyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl; and
   t is 0-4.

In some embodiments, t is 0 to 5. In some embodiments, t is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, t is 0, 1, 2, 3, 4, or 5.

In some embodiments, R¹ and R² are each independently hydrogen, or C₁-C₆ alkyl; R³ and R⁴ are each independently hydrogen or C₁-C₆ alkyl; R⁵ is hydrogen or C₁-C₆ alkyl; and R⁶ and R⁷ are each independently halogen, or C₁-C₆ alkyl.

In some embodiments, R¹ and R² are each hydrogen; R³ and R⁴ are each hydrogen; and R⁵ is hydrogen. In some embodiments, R¹ and R² are each hydrogen; R³ and R⁴ are each hydrogen; R⁵ is hydrogen; and t is 0.

In some embodiments, linker Z which connects residues X and B, comprises Z¹; wherein Z¹ is

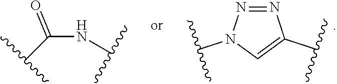 [SMILES: *NC(*)=O ; *c1cn(*)nn1]

In some embodiments, Z¹ is

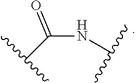 [SMILES: *NC(*)=O]

In some embodiments, Z is

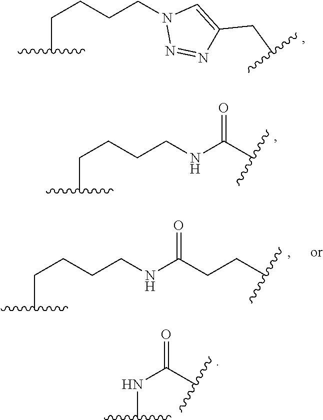 [SMILES: *CCCCn1cc(C*)nn1 ; *CCCCNC(*)=O ; *CCCCNC(=O)CC* ; *NC(*)=O]

In some embodiments, linker Z which connects residues X and B, comprises Z¹; wherein Z¹ is

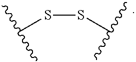 [SMILES: *SS*]

In some embodiments, Z is

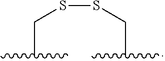 [SMILES: *CSSC*]

In some embodiments, linker Z which connects residues X and B, comprises Z¹; wherein Z¹ is a non-covalent linkage. In some embodiments, Z¹ is a metal chelate, a salt bridge, or nucleic acid hybridization.

In some embodiments, linker Z which connects residues X and B, comprises Z¹; wherein Z¹ is a metal chelate. In some embodiments, Z¹ is

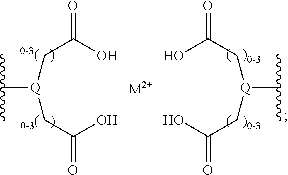

wherein Q is N or CH, and M²⁺ is a divalent metal cation. In some embodiments, M²⁺ is Mg²⁺, Cu²⁺, Ni²⁺, Zn²⁺ or Co²⁺. In some embodiments, M²⁺ is Mg²⁺ or Zn²⁺.

In some embodiments, linker Z which connects residues X and B, comprises Z¹; wherein Z¹ is a nucleic acid hybridization. In some embodiments, Z¹ is

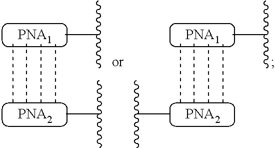

wherein PNA₁ and PNA₂ are polynucleic acids with complementary base pair sequences.

In some embodiments, the peptide of Formula (I) of the at least one peptide feature is synthesized from a functionalized peptide of Formula (II):

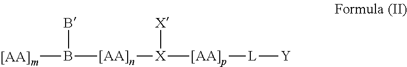

wherein,
each AA is a natural or unnatural amino acid residue;
m, n, and p are each independently an integer from 0 to 100;
X is a natural or unnatural amino acid residue which comprises cyclic precursor X′;
B is an acid residue which comprises cyclic precursor B′;
   wherein when m is not 0, B is a natural or unnatural amino acid residue;
   wherein each AA, B, and X residue is connected to the neighboring residues through peptide bonds;
L is a tether;
Y is a point of connection connecting the tether to a solid support having a reactive surface; and
wherein X′ and B′ are complementary groups that combine to form Z¹; or
X′ and B′ are groups that each combine with a third group Z² to form Z¹.

In some embodiments, X′ and B′ of Formula (II) are each selected from the group consisting of a thiol, an amine, a carboxylic acid, a haloacetate, a haloalkane, a dihaloalkane, an alkyne, an azide, an alkene, a natural amino acid side chain, an unnatural amino acid side chain, an N-terminal amino group, and a C-terminal carboxyl group.

In some embodiments, X of Formula (II) comprises a sidechain comprising X′. In some embodiments, X of Formula (II) comprises X′ at the C-terminal head of X. In some embodiments, wherein X is linked to [AA]ₚ-L- through an amino acid sidechain.

In some embodiments, B of Formula (II) comprises a sidechain comprising B′. In some embodiments, B of Formula (II) comprises B′ at the N-terminal tail of B. In some embodiments, B of Formula (II) is a natural or unnatural amino acid.

In some embodiments, when m of Formula (II) is 0, B is a natural or unnatural amino acid, or

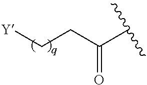

wherein q is 1-4. In some embodiments, q is 1 to 4. In some embodiments, q is 1 to 2, 1 to 3, 1 to 4, 2 to 3, 2 to 4, or 3 to 4. In some embodiments, q is 1, 2, 3, or 4.

In some embodiments, B′ of Formula (II) is

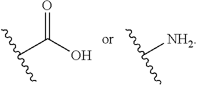

In some embodiments, X of Formula (II) is a natural or unnatural amino acid wherein X′ is

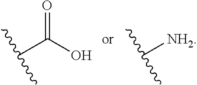

In some embodiments, one of X and B of Formula (II) is a suitably modified lysine, ornithine, diaminopropionic, or diaminobutyric acid.

In some embodiments, one of X and B of Formula (II) is a suitably modified aspartic or glutamic acid.

In some embodiments, X′ of Formula (II) is

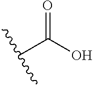 [SMILES: *C(=O)O]

and B′ of the at least one peptide feature is

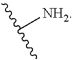 [SMILES: *N]

In some embodiments, B′ of Formula (II) is

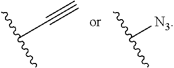

In some embodiments, X′ of Formula (II) is

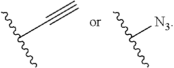

In some embodiments, B′ of Formula (II) is

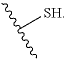 [SMILES: *S]

In some embodiments, X′ of Formula (II) is

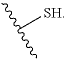 [SMILES: *S]

In some embodiments, for a given peptide feature, at least 80% of cyclic precursors X′ and B′ of Formula (II) have combined to form Z¹. In some embodiments, at least 85%, 90%, 95%, or 97% of cyclic precursors X′ and B′ of Formula (II) have combined to form Z¹.

In some embodiments, the % of cyclic precursors X′ and B′ that have combined to form Z¹ is determined by MALDI analysis.

In some embodiments, the % of cyclic precursors X′ and B′ that have combined to form Z¹ is determined by:
(a) labeling any uncombined X′ or B′ with an affinity handle or a reporter probe;
(b) detecting any uncombined X′ or B′.

In some embodiments, the affinity handle is biotin. In some embodiments, the determination of the % of cyclic precursors X′ and B′ that have combined to form Z¹ further comprises a step wherein a fluorescently labeled streptavidin is contacted with the biotin affinity handle. In some embodiments, the reporter probe is a fluorescent dye comprising a functional group that reacts specifically with uncombined X′ or B′, or both. In some embodiments, any uncombined X′ or B′ in a given peptide feature is detected by fluorescence.

In some embodiments, m, n, and p of the at least one cyclic peptide feature are each independently 0-30.

In some embodiments, n of the at least one cyclic peptide feature is 0-30. In some embodiments, n of the at least one cyclic peptide feature is 0 to 1, 0 to 2, 0 to 3, 0 to 4, 0 to 5, 0 to 6, 0 to 7, 0 to 8, 0 to 9, 0 to 10, 0 to 11, 0 to 12, 0 to 13, 0 to 14, 0 to 15, 0 to 16, 0 to 17, 0 to 18, 0 to 19, 0 to 20, 0 to 21, 0 to 22, 0 to 23, 0 to 24, 0 to 25, 0 to 26, 0 to 27, 0 to 28, 0 to 29, or 0 to 30. In some embodiments, n of the at least one cyclic peptide feature is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n of the at least one cyclic peptide feature is 3.

In some embodiments, m of the at least one cyclic peptide feature is 0-30. In some embodiments, m of the at least one cyclic peptide feature is 0 to 1, 0 to 2, 0 to 3, 0 to 4, 0 to 5, 0 to 6, 0 to 7, 0 to 8, 0 to 9, 0 to 10, 0 to 11, 0 to 12, 0 to 13, 0 to 14, 0 to 15, 0 to 16, 0 to 17, 0 to 18, 0 to 19, 0 to 20, 0 to 21, 0 to 22, 0 to 23, 0 to 24, 0 to 25, 0 to 26, 0 to 27, 0 to 28, 0 to 29, or 0 to 30. In some embodiments, n of the at least one cyclic peptide feature is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, m of the at least one cyclic peptide feature is 0-18. In some embodiments, m of the at least one cyclic peptide feature is 0-2.

In some embodiments, p of the at least one cyclic peptide feature is 0-30. In some embodiments, p of the at least one cyclic peptide feature is 0 to 1, 0 to 2, 0 to 3, 0 to 4, 0 to 5, 0 to 6, 0 to 7, 0 to 8, 0 to 9, 0 to 10, 0 to 11, 0 to 12, 0 to 13, 0 to 14, 0 to 15, 0 to 16, 0 to 17, 0 to 18, 0 to 19, 0 to 20, 0 to 21, 0 to 22, 0 to 23, 0 to 24, 0 to 25, 0 to 26, 0 to 27, 0 to 28, 0 to 29, or 0 to 30. In some embodiments, p of the at least one cyclic peptide feature is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments, the peptide array further comprises at least one linear peptide feature. In some embodiments, the at least one linear peptide feature comprises an amino acid sequence substantially the same as that of the at least one cyclic peptide feature. In some embodiments, the at least one linear peptide feature comprises an amino acid sequence with greater than 80%, 85%, 90%, 95%, or 98% sequence homology with the at least one cyclic peptide feature. In some embodiments, the at least one linear peptide feature has the same amino acid sequence as the at least one cyclic peptide feature, except that it comprises amino acids at X and B that cannot combine to form linkage Z as in the at least one cyclic peptide feature.

In some embodiments, the peptide array further comprises other cyclic peptide features of Formula (I), wherein each X—Z—B of the other cyclic peptide features is the same as X—Z—B of the at least one cyclic peptide feature.

In some embodiments, the peptide array further comprises other cyclic peptide features of Formula (I), wherein each X—Z—B of the other cyclic peptide features is the same as or different than the X—Z—B of the at least one cyclic peptide feature.

In some embodiments, at least one of said peptides on the array comprises a disease-related peptide. In some embodiments, at least one of said peptides comprises a disease-related peptide in a reversed order. In some embodiments, at least one of said peptides comprises a disease-related peptide in a scrambled or randomized order. In some embodiments, at least one of said peptides has greater than 80%, greater than 85%, greater than 90%, greater than 95%, or greater than 98% sequence homology to a disease-related peptide. In some embodiments, at least one of said peptides has greater than 80%, greater than 85%, greater than 90%, greater than 95%, or greater than 98% sequence homology to a disease-related peptide in a reversed order. In some embodiments, at least one of said peptides has greater than 80%, greater than 85%, greater than 90%, greater than 95%, or greater than 98% sequence homology to a disease-related peptide in a scrambled or randomized order. In some embodiments, the disease-related peptide is an epitope, a receptor ligand, a receptor agonist, a receptor antagonist, an enzyme substrate, and enzyme inhibitor, an inhibitor of a protein-protein interaction.

In some embodiments, at least one of said peptides on the array is a random peptide sequence.

In some embodiments, the peptide features are 5 to 100 amino acids in length. In some embodiments, the peptide features are 5 to 30 amino acids in length.

In some embodiments, said array comprises at least about 10,000, 300,000, or 1 million peptide features. In some embodiments, said array comprises at least about 10,000, 300,000, 1 million, 2 million, or 3 million peptide features. In some embodiments, said array comprises about 16,000 peptide features. In some embodiments, said array comprises about 3.3 million peptide features.

In some embodiments, said array comprises at least about 10,000, 300,000, or 1 million peptide features per 1 cm². In some embodiments, said array comprises at least about 10,000, 50,000, 100,000, 200,000, 300,000, 400,000, 500,000, 600,000, 700,000, 800,000, 900,000, or 1 million peptide features per 1 cm². In some embodiments, said array comprises about 800,000 peptide features per 1 cm².

In some embodiments, said solid support is a substrate, bead, polymer, or chromatographic packing material. In some embodiments, said solid support is a Si/SiO₂ wafer.

In some embodiments, said peptides on the array are synthesized in situ.

In some instances, such array molecules include the in situ synthesis of large synthetic peptide arrays. In some embodiments, a molecule in an array is a mimotope, a molecule that mimics the structure of an epitope and is able to bind an epitope-elicited antibody with relative specificity and sensitivity. In some embodiments, an array of the invention is a peptide array comprising diverse peptide sequences. In some embodiments, the diverse peptide sequences may be derived from a proteome library, for example, from a specific organism (see, e.g., Mycobacterium tuberculosis (Mtb) proteome library (Schubert et al., Cell Host Microbe (2013) 13(5):602-12), or organelle (see, e.g., Mitochondrial (Mtd) proteome library (Calvo and Mootha, Annu. Rev. Genomics (2010) 11:25-44), and the like. In other instances, an array of the invention is a peptide array comprising peptide sequences that are generated independent of any known target or sequence, including for example random or semi-random peptide sequences.

In yet other embodiments, the diverse peptide sequences may be derived from a set of all known combinations of amino acids, for example at least 100% of all possible tetramers, at least 90% of all possible tetramers, at least 85% of all possible tetramers, at least 80% of all possible tetramers, at least 75% of all possible tetramers, at least 70% of all possible tetramers, at least 65% of all possible tetramers, at least 60% of all possible tetramers, at least 55% of all possible tetramers, at least 50% of all possible tetramers, at least 45% of all possible tetramers, at least 40% of all possible tetramers, at least 35% of all possible tetramers, at least 30% of all possible tetramers, or at least 25% of all possible tetramers. In still other embodiments, the diverse peptide sequences may be derived from a set of all possible pentamers, for example, at least 100% of all possible pentamers, at least 95% of all possible pentamers, at least 90% of all possible pentamers, at least 85% of all possible pentamers, at least 80% of all possible pentamers, at least 75% of all possible pentamers, at least 70% of all possible pentamers, at least 65% of all possible pentamers, at least 60% of all possible pentamers, at least 55% of all possible pentamers, at least 50% of all possible pentamers, at least 45% of all possible pentamers, at least 40% of all possible pentamers, at least 35% of all possible pentamers, at least 30% of all possible pentamers or at least 25% of all possible pentamers. In yet other embodiments, the diverse peptide sequences of an array may be derived from a set of amino acid combinations, for example from 25%-100% of all possible hexamers, from 25%-100% of all possible septamers, from 25%-100% of all possible octamers, from 25%-100% of all possible nonamers or from 25%-100% of all possible decamers, or combinations thereof. In some embodiments, the diverse peptide sequences of an array may be derived from a set of amino acid combinations, for example from 25%-100% of all possible hexamers.

Representation of the diverse peptide sequences is only limited by the size of the array. Accordingly, large arrays, for example, at least 1 million, at least 2 million, at least 3 million, at least 4 million, at least 5 million, at least 6 million, at least 7 million, at least 8 million, at least 9 million, at least 10 million or more peptides can be used with the methods, systems and assays disclosed herein. Alternatively or additionally, multiple substantially non-overlapping peptide libraries/arrays may be synthesized. Focused or directed arrays, which comprise peptides that may be directed to specific binding sequences and derivatives or portions thereof, of about 10,000, about 15,000, about 20,000, about 25,000, or about 30,000 may be synthesized to cover the substitution space surrounding the peptide sequence or motifs(s) recognized by the biological sample, peptides, or proteins. In some embodiments, focused arrays of about 15,000, about 16,000, about 17,000, about 18,000, about 19,000, about 20,000, about 21,000, about 22,000, about 23,000, about 24,000, or about 25,000 peptides may be synthesized to cover the substitution space surrounding the peptide sequence or motifs(s) recognized by the biological sample, peptides, or proteins. In some embodiments, focused arrays of about 16,000 peptides may be synthesized to cover the substitution space surrounding the peptide sequence or motifs(s) recognized by the biological sample, peptides, or proteins.

In some embodiments, the individual peptides on the array are of variable and/or different lengths. In some embodiments, the peptides are between about 5-30 amino acids in length, between about 5-25 amino acids in length, between about 5-20 amino acids in length, or between about 5-18 amino acids in length, or between about 5-15 amino acids in length, or between about 5-14 amino acids in length. In other embodiments, the peptides are at least 5 amino acids, at least 6 amino acids, at least 7 amino acids, at least 8 amino acids, at least 9 amino acids, at least 10 amino acids, at least 11 amino acids, at least 12 amino acids, at least 13 amino acids, at least 14 amino acids, at least 15 amino acids in length. In still other embodiments, the peptides are not more than 15 amino acids, not more than 14 amino acids, not more than 13 amino acids, not more than 12 amino acids, not more than 11 amino acids, not more than 10 amino acids, not more than 9 amino acids or not more than 8 amino acids in length. In still other embodiments, the peptides on the array have an average length of about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, or about 15 amino acids.

In yet other embodiments, the amino acid building blocks for the peptides on the array comprise all natural amino acids. In other embodiments, the amino acid building blocks for the peptides on the array comprise non-natural or synthetic amino acids. In yet other embodiments, only 19 amino acids are used as the building blocks for synthesizing the peptides on the array. In still other embodiments, only 18 amino acids, only 17 amino acids, only 16 amino acids, only 15 amino acids, or only 14 amino acids are used as the building blocks for synthesizing the peptides on the array. In some embodiments, cysteine is omitted during peptide synthesis. In other embodiments, methionine is omitted during peptide synthesis. In still other embodiments, isoleucine is omitted during peptide synthesis. In yet other embodiments, threonine is omitted during peptide synthesis. In still other embodiments, any amino acid can be omitted during peptide synthesis. In some embodiments, amino acids can be co-coupled during peptide synthesis. For example, in some embodiments aspartic acid and glutamic acid are applied to the array in the same coupling step.

In some embodiments, a molecule in an array is a conformationally constrained peptide. In some embodiments, a molecule in an array is a cyclic peptide. In some aspects, this conformational constraint is a covalent bond. In some embodiments, the conformational constraint is a disulfide, lactam, triazole, thioether, or the like. In some embodiments, the conformational constraint is a non-covalent interaction. In some embodiments, the conformational constraint is a metal chelate, salt bridge, hydrogen bond, or intramolecular nucleic acid hybridization. In some embodiments, the conformational constraint is a bond between a sidechain of one amino acid and the sidechain of another amino acid (sidechain-to-sidechain, S2S). In other embodiments, the conformational constraint is a bond between a sidechain of one amino acid and the C-terminal head or N-terminal tail of another amino acid (sidechain-to-tail, S2T, or head-to-sidechain (H2S). In still other embodiments, the conformational constraint is a bond between the C-terminal head of one amino acid and the N-terminal tail of another amino acid (head-to-tail, H2T).

In some embodiments, an array comprises peptide features that have not been cyclized. In some embodiments, an array comprises peptide features that have been cyclized. In some embodiments, an array comprises some peptide features that have been cyclized and some peptide features that have not been cyclized. In some embodiments, the amount of cyclized peptide molecules in a given peptide feature is at least 50%, at least 55%, at least 60%, at least 65%, at least 70%, at least 75%, at least 80%, at least 85%, at least 90%, at least 95%, or at least 97%. In other embodiments, the amount of cyclized peptide molecules in a given peptide feature is at least 10%, at least 15%, at least 20%, at least 25%, at least 30%, at least 35%, at least 40%, or at least 45%.

In some embodiments, an array of the invention is a peptide array comprising a focused or limited set of peptide sequences, all derived from an input amino acid or peptide sequence, or an input amino acid or peptide motif. One or more peptide arrays may be used with the methods, systems and assays disclosed herein, including a diverse or semi-random peptide array and/or a focused or limited set of peptide sequences. For example, the methods, systems and assays disclosed herein may utilize both a diverse set of peptides and a focused or limited set of peptides are chosen. The peptide arrays may be used either in parallel or sequentially with a biological sample as disclosed herein. For example, a diverse peptide array may be used initially, and at least one motif (either sequence or structure-based) or sequence is obtained for a monoclonal antibody, for example, with an unknown binding profile. The identified motif or sequence may be then used as the input sequence for the creation of at least one focused or limited set of peptide sequences, and assays performed as described herein. Using the methods, systems and arrays described herein, multiple focused or limited set of peptide arrays may be used to characterize antibody binding for the unknown monoclonal antibody.

Nearly all therapeutic antibody screens incorporate some level of epitope mapping and epitope binning on a select number of leads and these data drive decisions on which leads move forward into the development pipeline. Epitope mapping studies commonly utilize systematic overlapping sequences of peptides to determine the amino acids responsible for the antibody-target interaction. Epitope binning studies map the epitopes of several lead antibodies and then bin the antibodies by their binding affinity/kinetics towards identified epitopes. Epitope binning studies assist in identifying lead antibodies with different epitope reactivity and potentially different modes-of-action and off-target effects. Typically epitope binning and mapping characterizations are done using synthesized libraries of targeted peptide sequences related to known epitope(s), which limits analyses to a few thousand targeted interactions (e.g. 10 lead antibodies vs. 100 peptides) due to limited analysis throughput and the high cost of purified synthetic peptide libraries. Characterization of such a small number of antibody-target interactions allows many off-target and/or low-affinity interactions to go undetected which increases failure rates of candidates late in the development pipeline.

A common weakness of current epitope mapping/binning platforms is severely limited antibody-epitope interaction analysis throughput relative to the total number of possible interactions. This analytical throughput limitation limits antibody discovery scientists to reduce the number of leads selected for further development. As a result, the reduced number of leads increases the risk of late-stage antibody therapeutic candidate failure. Risks associated with limited analytical throughput are increasing with the advent of multi-specific antibody screens that require selection of more numerous lead antibodies to identify candidates with particular multi-specificity relevant to the target disease and minimal off-target effects.

The technologies disclosed herein include an in situ peptide array synthesis platform that produces conformationally constrained peptide array-based libraries on silicon wafers. By utilizing an orthogonal protecting group strategy, linear peptides can be synthesized on the array followed by a final cyclization step. In some embodiments disclosed herein, the cyclization is between a sidechain of a first amino acid of the linear peptide and a sidechain of a second amino acid of the same linear peptide, referred to as sidechain-to-sidechain cyclization (S2S). In other embodiments, the cyclization is between a sidechain of a first amino acid of the linear peptide and the N-terminal tail of a second amino acid of the same linear peptide, referred to as sidechain-to-tail cyclization (S2T). In other embodiments, the cyclization is between a C-terminal head of a first amino acid of the linear peptide and the N-terminal tail of a second amino acid of the same linear peptide, referred to as head-to-tail cyclization (H2T). In other embodiments, the cyclization is between a sidechain of a first amino acid of the linear peptide and the C-terminal head of a second amino acid of the same linear peptide, referred to as sidechain-to-tail cyclization (H2S).

In one aspect described herein, the S2S cyclization is performed by a 3+2 cycloaddition, or Click, reaction and the product is a triazole, as shown in Scheme 1.

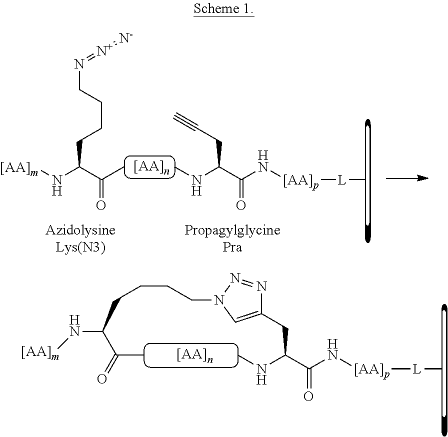

In another aspect described herein, the S2S cyclization is performed by amide bond formation and the product is a lactam, as shown in Scheme 2.

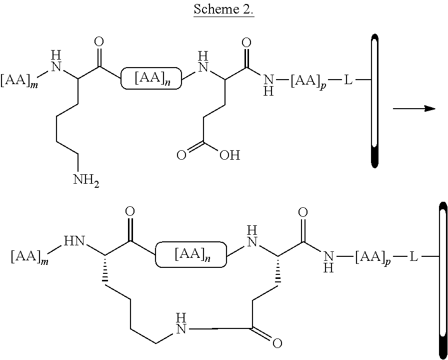

In another aspect described herein, the H2T or H2S cyclization is performed by amide bond formation and the product is a lactam, as shown in Scheme 3.

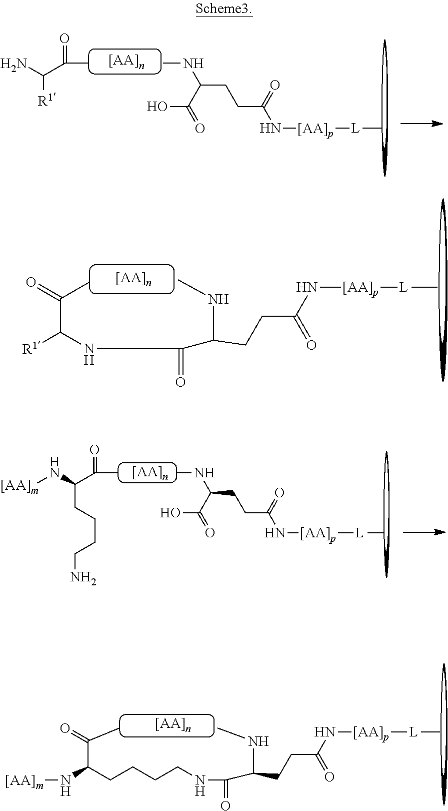

In some embodiments, the amide coupling reagent is DCC, DIC, EDC, BOP, PyBOP, PyBrOP, PyAOP, PyOxim, DEPBT, TBTU, HCTU, HDMC, COMU, HBTU, HATU, TBTU, TATU, TOTT, TFFH, EEDQ, T3P, DMTMM, CDI, BTC, or the like. In some embodiments, the amide coupling reagent is PyBOP.

In another aspect described herein, the S2S cyclization is performed by disulfide formation by metal catalysis, as shown in Scheme 4.

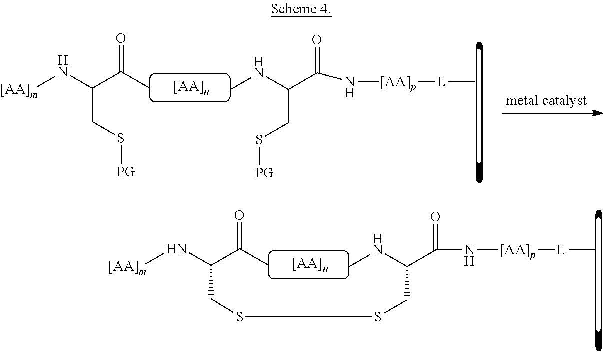

In some embodiments, the metal catalyst both deprotects the free cysteine thiol and forms the disulfide in a single step. In some embodiments, the cysteine thiols are protected by a substituted benzyl group. In some embodiments, the cysteine thiols are protected by a 4-methyl-benzyl protecting group. In some embodiments, the metal catalyst is a transition metal catalyst. In some embodiments, the metal catalyst is a post-transition metal catalyst. In some embodiments, the metal catalyst is a Tl(III) salt. In some embodiments, the metal catalyst is Tl(OTFA)₃.

In another aspect described herein, the S2S cyclization is performed by disulfide formation by oxidation of free cysteine thiols, as shown in Scheme 5.

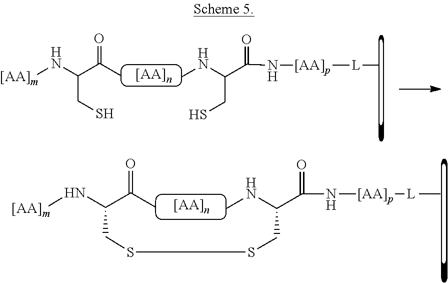

In some embodiments, the free cysteine thiols are oxidized by a metal catalyst. In some embodiments, the free cysteine thiols are oxidized by air oxidation. In some embodiments, the free cysteine thiols are oxidized by a soluble oxidant or an oxidation buffer, such as, for example, oxidized glutathione, cystine, or 5,5′-dithiobis-(2-nitrobenzoic acid) (DTNB).

In some embodiments, the cysteine thiols are protected by a protecting group which is removed prior to the oxidation step. In some embodiments, the cysteine thiols are protected by a substituted benzyl group. In some embodiments, the cysteine thiols are protected by a 4-methyl-benzyl, or a 4-methoxy-benzyl protecting group. In some embodiments, the thiol protecting group is removed by treatment with a Lewis acid or a metal catalyst. In some embodiments, the thiol protecting group is removed by treatment with TMS-OTf, TFA, or Tl(OTFA)₃, or a combination thereof.

In some embodiments, arrays with chemical libraries produced by the technologies disclosed herein are used for immune-based diagnostic assays, for example called immunosignature assays. Using a patient's antibody repertoire from a drop of blood bound to the arrays, a fluorescence binding profile image of the bound array provides sufficient information to classify disease vs. healthy.

In another aspect, disclosed herein is a method of synthesizing a peptide array, comprising at least one cyclic peptide feature which comprises peptides of Formula (I):

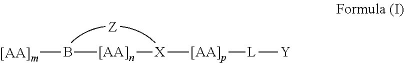

wherein,
   each AA is independently a natural or unnatural amino acid residue;
   m, n, and p are each independently an integer from 0 to 100;
   X is a natural or unnatural amino acid residue;
   B is an acid residue; wherein when m is not 0, B is a natural or unnatural amino acid residue;
      wherein each AA, B, and X residue is connected to adjacent residues through peptide bonds;
   Z is a linker that connects residues X and B;
      wherein Z is

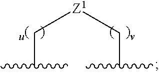

u and v are independently 0-5; and Z′ is a covalent or non-covalent linkage;
L is a tether that is optionally present; and
Y is a point of connection connecting the tether to a solid support having a reactive surface;
the method comprising the step of:
(b) reacting a functionalized peptide of Formula (II):

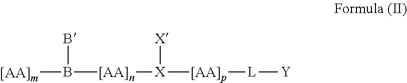

wherein,
   wherein X′ and B′ are complementary groups that combine to form Z¹; or
   X′ and B′ are groups that each combine with a third group Z² to form Z¹;
under conditions that cause linkage Z¹ to form.

In some embodiments, u is 0 to 5. In some embodiments, u is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, u is 0, 1, 2, 3, 4, or 5.

In some embodiments, v is 0 to 5. In some embodiments, v is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, v is 0, 1, 2, 3, 4, or 5.

It is the intention of this disclosure for each Z to be connecting B and X in either direction. In some embodiments, Z as pictured connects to B and X from left-to-right. In some embodiments, Z as pictured connects to B and X from right-to-left.

In some embodiments, each peptide of Formula (I) is independently represented by Formula (Ia) or Formula (Ib):

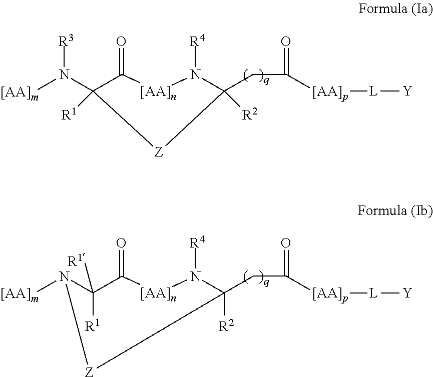

wherein:
   q is 0-5;
   R¹ and R² are each independently hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl;
   R^{1′} is a natural or unnatural amino acid sidechain;
   R³ and R⁴ are each independently hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl.

In some embodiments, linker Z which connects residues X and B, comprises Z¹; wherein Z¹ is a covalent linkage. In some embodiments, Z¹ is:

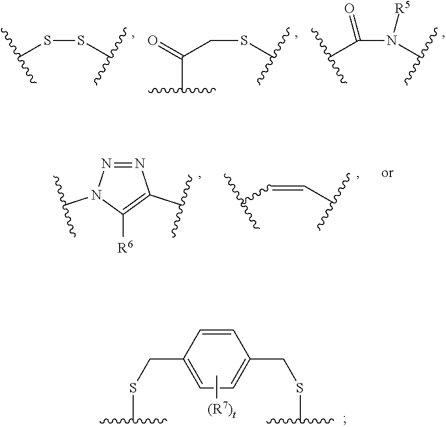

wherein:
   R⁵ is hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl;
   R⁶ and R⁷ are each independently halogen, hydroxy, C₁-C₆ alkyl, C₁-C₆ alkoxyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl; and
   t is 0-4.

In some embodiments, t is 0 to 5. In some embodiments, t is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, t is 0, 1, 2, 3, 4, or 5.

In some embodiments, R¹ and R² are each independently hydrogen, or C₁-C₆ alkyl; R³ and R⁴ are each independently hydrogen or C₁-C₆ alkyl; R⁵ is hydrogen or C₁-C₆ alkyl; and R⁶ and R⁷ are each independently halogen, or C₁-C₆ alkyl.

In some embodiments, R¹ and R² are each hydrogen; R³ and R⁴ are each hydrogen; and R⁵ is hydrogen. In some embodiments, R¹ and R² are each hydrogen; R³ and R⁴ are each hydrogen; R⁵ is hydrogen; and t is 0.

In some embodiments, linker Z which connects residues X and B, comprises Z¹; wherein Z¹ is

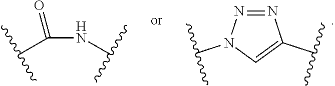 [SMILES: *NC(*)=O ; *c1cn(*)nn1]

In some embodiments, Z¹ is

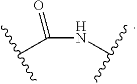 [SMILES: *NC(*)=O]

In some embodiments, Z is

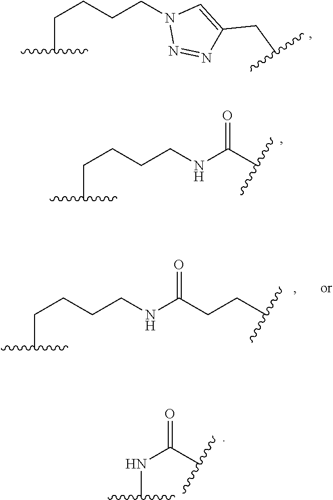 [SMILES: *CCCCn1cc(C*)nn1 ; *CCCCNC(*)=O ; *CCCCNC(=O)CC* ; *NC(*)=O]

In some embodiments, linker Z which connects residues X and B, comprises Z¹; wherein Z¹ is

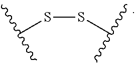 [SMILES: *SS*]

In some embodiments, Z is

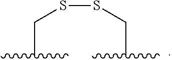 [SMILES: *CSSC*]

In some embodiments, linker Z which connects residues X and B, comprises Z¹; wherein Z¹ is a non-covalent linkage. In some embodiments, Z¹ is a metal chelate, a salt bridge, or nucleic acid hybridization.

In some embodiments, linker Z which connects residues X and B, comprises Z¹; wherein Z¹ is a metal chelate. In some embodiments, Z¹ is

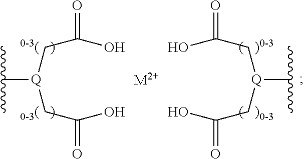

wherein Q is N or CH, and M²⁺ is a divalent metal cation. In some embodiments, M²⁺ is Mg²⁺, Cu²⁺, Ni²⁺, Zn²⁺ or Co²⁺. In some embodiments, M²⁺ is Mg²⁺ or Zn²⁺.

In some embodiments, linker Z which connects residues X and B, comprises Z¹; wherein Z¹ is a nucleic acid hybridization. In some embodiments, Z¹ is

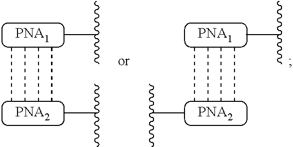

wherein PNA₁ and PNA₂ are polynucleic acids with complementary base pair sequences.

In some embodiments, X′ and B′ of Formula (II) are each selected from the group consisting of a thiol, an amine, a carboxylic acid, a haloacetate, a haloalkane, a dihaloalkane, an alkyne, an azide, an alkene, a natural amino acid side chain, an unnatural amino acid side chain, an N-terminal amino group, and a C-terminal carboxyl group.

In some embodiments, X of Formula (II) comprises a sidechain comprising X′. In some embodiments, X of Formula (II) comprises X′ at the C-terminal head of X. In some embodiments, X is linked to [AA]ₚ-L- through an amino acid sidechain.

In some embodiments, B of Formula (II) comprises a sidechain comprising B′. In some embodiments, B of Formula (II) comprises B′ at the N-terminal tail of B. In some embodiments, B of Formula (II) is a natural or unnatural amino acid.

In some embodiments, m of Formula (II) is 0, B is a natural or unnatural amino acid, or

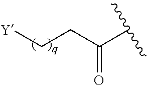

wherein q is 1-4. In some embodiments, q is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, q is 0, 1, 2, 3, 4, or 5.

In some embodiments, B′ of Formula (II) is

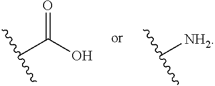

In some embodiments, X of Formula (II) is a natural or unnatural amino acid wherein X′ is

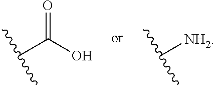

In some embodiments, one of X and B of Formula (II) is a suitably modified lysine, ornithine, diaminopropionic, or diaminobutyric acid. In some embodiments, one of X and B of Formula (II) is a suitably modified aspartic or glutamic acid.

In some embodiments, one of X′ and B′ of the functionalized peptide of Formula (II) is

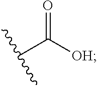 [SMILES: *C(=O)O]

the other of X′ and B′ of the functionalized peptide of Formula (II) is

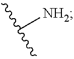 [SMILES: *N]

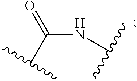 [SMILES: *NC(*)=O]

and conditions that cause linkage Z¹ to form comprise: i) contacting the peptide of Formula (II) with a suitable amide coupling reagent and a suitable base in a suitable solvent In some embodiments, X′ is

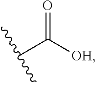 [SMILES: *C(=O)O]

### and B′ is

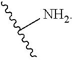 [SMILES: *N]

In some embodiments, the amide coupling reagent is DCC, DIC, EDC, BOP, PyBOP, PyBrOP, PyAOP, PyOxim, DEPBT, TBTU, HCTU, HDMC, COMU, HBTU, HATU, TBTU, TATU, TOTT, TFFH, EEDQ, T3P, DMTMM, CDI, BTC, or the like. In some embodiments, the amide coupling reagent is PyBOP.

In some embodiments, one of X′ and B′ of the functionalized peptide of Formula (II) is

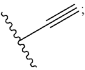 [SMILES: *C#C]

the other of X′ and B′ of the functionalized peptide of Formula (II) is

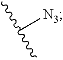 [SMILES: *N=[N+]=[N-]]

### Z¹ is

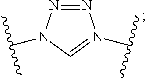

and conditions that cause linkage Z¹ to form comprise: i) contacting the peptide of Formula (II) with a solution comprising Cu(I). In some embodiments, the solution comprising Cu(I) is generated in situ from a Cu(II) salt. In some embodiments, the Cu(II) salt is CuSO₄, CuBr₂, CuCl₂, CuOH₂, or Cu(NO₃)₂. In some embodiments, the Cu(II) salt is CuSO₄.

In some embodiments, one of X′ and B′ of the functionalized peptide of Formula (II) is

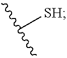 [SMILES: *S]

the other of X′ and B′ of the functionalized peptide of Formula (II) is

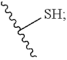 [SMILES: *S]

### Z¹ is

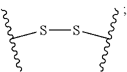 [SMILES: *SS*]

and conditions that cause linkage Z¹ to form comprise: i) contacting the peptide of Formula (II) with an oxidant. In some embodiments, the oxidant is atmospheric oxygen. In some embodiments, the oxidation is spontaneous. In some embodiments, the oxidant is an exogenous disulfide molecule such as oxidized glutathione, cystine, or 5,5′-dithiobis-(2-nitrobenzoic acid) (DNTB). In some embodiments, the oxidant is H₂O₂, I₂, a Cu(II) salt, or a Fe(III) salt. In some embodiments, the oxidant is a metal oxidant. In some embodiments, the oxidant is a Tl(III) salt.

In some embodiments, one of X′ and B′ of the functionalized peptide of Formula (II) is

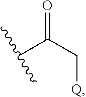

wherein Q is Cl or Br; the other of X′ and B′ of the functionalized peptide of Formula (II) is

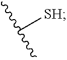 [SMILES: *S]

### and Z¹ is

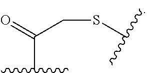 [SMILES: *SCC(*)=O]

In some embodiments, one of X′ and B′ of the functionalized peptide of Formula (II) is

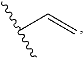

wherein W is Cl or Br; the other of X′ and B′ of the functionalized peptide of Formula (II) is

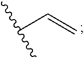 [SMILES: *C=C]

### Z¹ is

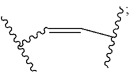

and conditions that cause linkage Z¹ to form comprise: i) contacting the peptide of Formula (II) with a metal catalyst suitable for olefin metathesis. In some embodiments, the metal catalyst is a Pd, Mo, Ru, Pt, W, Ti, or the like.

In some embodiments, one of X′ and B′ of the functionalized peptide of Formula (II) is

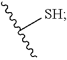 [SMILES: *S]

the other of X′ and B′ of the functionalized peptide of Formula (II) is

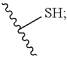 [SMILES: *S]

### Z¹ is

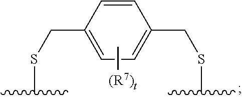

and conditions that cause linkage Z¹ to form comprise: i) contacting the peptide of Formula (II) with a dielectrophile of the structure

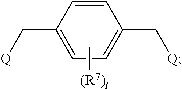

wherein Q is a suitable leaving group. In some embodiments, Q is a halogen. In some embodiments, Q is Br. In some embodiments,

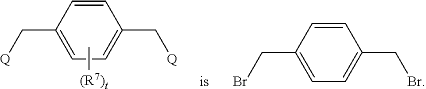

In some embodiments, one of X′ and B′ of the functionalized peptide of Formula (II) is

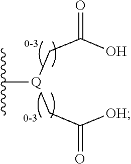

the other of X′ and B′ of the functionalized peptide of Formula (II) is

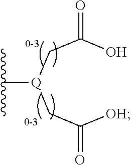

### Z¹ is

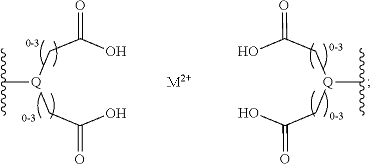

wherein Q is N or CH, and M²⁺ is a divalent metal cation; and conditions that cause linkage Z¹ to form comprise: i) contacting the peptide of Formula (II) with the divalent metal cation, M²⁺. In some embodiments M²⁺ is Mg²⁺, Cu²⁺, Ni²⁺, Zn²⁺ Co²⁺. In some embodiments, M²⁺ is Mg²⁺ or Zn²⁺.

In some embodiments, one of X′ and B′ of the functionalized peptide of Formula (II) is

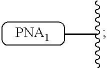

the other of X′ and B′ of the functionalized peptide of Formula (II) is

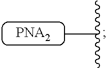

### and Z¹ is

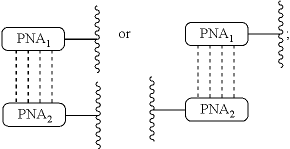

wherein PNA₁ and PNA₂ are polynucleic acids with complementary base pair sequences.

In some embodiments, m, n, and p of the at least one cyclic peptide feature are each independently 0-30.

In some embodiments, n of the at least one cyclic peptide feature is 0-30. In some embodiments, n of the at least one cyclic peptide feature is 0 to 1, 0 to 2, 0 to 3, 0 to 4, 0 to 5, 0 to 6, 0 to 7, 0 to 8, 0 to 9, 0 to 10, 0 to 11, 0 to 12, 0 to 13, 0 to 14, 0 to 15, 0 to 16, 0 to 17, 0 to 18, 0 to 19, 0 to 20, 0 to 21, 0 to 22, 0 to 23, 0 to 24, 0 to 25, 0 to 26, 0 to 27, 0 to 28, 0 to 29, or 0 to 30. In some embodiments, n of the at least one cyclic peptide feature is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n of the at least one cyclic peptide feature is 3.

In some embodiments, m of the at least one cyclic peptide feature is 0-30. In some embodiments, m of the at least one cyclic peptide feature is 0 to 1, 0 to 2, 0 to 3, 0 to 4, 0 to 5, 0 to 6, 0 to 7, 0 to 8, 0 to 9, 0 to 10, 0 to 11, 0 to 12, 0 to 13, 0 to 14, 0 to 15, 0 to 16, 0 to 17, 0 to 18, 0 to 19, 0 to 20, 0 to 21, 0 to 22, 0 to 23, 0 to 24, 0 to 25, 0 to 26, 0 to 27, 0 to 28, 0 to 29, or 0 to 30. In some embodiments, n of the at least one cyclic peptide feature is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, m of the at least one cyclic peptide feature is 0-18. In some embodiments, m of the at least one cyclic peptide feature is 0-2.

In some embodiments, p of the at least one cyclic peptide feature is 0-30. In some embodiments, p of the at least one cyclic peptide feature is 0 to 1, 0 to 2, 0 to 3, 0 to 4, 0 to 5, 0 to 6, 0 to 7, 0 to 8, 0 to 9, 0 to 10, 0 to 11, 0 to 12, 0 to 13, 0 to 14, 0 to 15, 0 to 16, 0 to 17, 0 to 18, 0 to 19, 0 to 20, 0 to 21, 0 to 22, 0 to 23, 0 to 24, 0 to 25, 0 to 26, 0 to 27, 0 to 28, 0 to 29, or 0 to 30. In some embodiments, p of the at least one cyclic peptide feature is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments, the peptide array further comprises at least one linear peptide feature. In some embodiments, the at least one linear peptide feature comprises an amino acid sequence substantially the same as that of the at least one cyclic peptide feature. In some embodiments, the at least one linear peptide feature comprises an amino acid sequence with greater than 80%, 85%, 90%, 95%, or 98% sequence homology with the at least one cyclic peptide feature. In some embodiments, the at least one linear peptide feature has the same amino acid sequence as the at least one cyclic peptide feature, except that it comprises amino acids at X and B that cannot combine to form linkage Z as in the at least one cyclic peptide feature.

In some embodiments, the peptide array further comprises other cyclic peptide features of Formula (I), wherein each X—Z—B of the other cyclic peptide features is the same as X—Z—B of the at least one cyclic peptide feature.

In some embodiments, the peptide array further comprises other cyclic peptide features of Formula (I), wherein each X—Z—B of the other cyclic peptide features is the same as or different than the X—Z—B of the at least one cyclic peptide feature.

In some embodiments, at least one of said peptides on the array comprises a disease-related peptide. In some embodiments, at least one of said peptides comprises a disease-related peptide in a reversed order. In some embodiments, at least one of said peptides comprises a disease-related peptide in a scrambled or randomized order. In some embodiments, at least one of said peptides has greater than 80%, greater than 85%, greater than 90%, greater than 95%, or greater than 98% sequence homology to a disease-related peptide. In some embodiments, at least one of said peptides has greater than 80%, greater than 85%, greater than 90%, greater than 95%, or greater than 98% sequence homology to a disease-related peptide in a reversed order. In some embodiments, at least one of said peptides has greater than 80%, greater than 85%, greater than 90%, greater than 95%, or greater than 98% sequence homology to a disease-related peptide in a scrambled or randomized order. In some embodiments, the disease-related peptide is an epitope, a receptor ligand, a receptor agonist, a receptor antagonist, an enzyme substrate, and enzyme inhibitor, an inhibitor of a protein-protein interaction.

In some embodiments, at least one of said peptides on the array is a random peptide sequence.

In some embodiments, the peptide features are 5 to 100 amino acids in length. In some embodiments, the peptide features are 5 to 30 amino acids in length.

In some embodiments, said array comprises at least about 10,000, 300,000, or 1 million peptide features. In some embodiments, said array comprises at least about 10,000, 300,000, 1 million, 2 million, or 3 million peptide features. In some embodiments, said array comprises about 16,000 peptide features. In some embodiments, said array comprises about 3.3 million peptide features.

In some embodiments, said array comprises at least about 10,000, 300,000, or 1 million peptide features per 1 cm². In some embodiments, said array comprises at least about 10,000, 50,000, 100,000, 200,000, 300,000, 400,000, 500,000, 600,000, 700,000, 800,000, 900,000, or 1 million peptide features per 1 cm². In some embodiments, said array comprises about 800,000 peptide features per 1 cm².

In some embodiments, said solid support is a substrate, bead, polymer, or chromatographic packing material. In some embodiments, said solid support is a Si/SiO₂ wafer.

In some embodiments, said peptides on the array are synthesized in situ.

In some embodiments, the method of synthesis further comprises the step: b) determining which peptide features of a peptide array have successfully cyclized after a cyclization step, and the % to which they have successfully cyclized. In some embodiments, for a given peptide feature, at least 80% of cyclic precursors X′ and B′ of Formula (II) have combined to form Z¹. In some embodiments, for a given peptide feature, at least 85%, 90%, 95%, or 97% of cyclic precursors X′ and B′ of Formula (II) have combined to form Z¹. In some embodiments, for a given peptide feature, at least 20%, 25%, 30%, 35%, 40%, 45%, 50%, 55%, 60%, 65%, 70%, or 75% of cyclic precursors X′ and B′ of Formula (II) have combined to form Z¹. In some embodiments, for a given peptide feature, at least 20% of cyclic precursors X′ and B′ of Formula (II) have combined to form Z¹.

### Determination of Cyclization Efficiency

One of the major deficiencies of conformationally constrained peptide arrays has been the inability to accurately measure the amount of each peptide feature that had successfully cyclized on the scale of the entire library. Without knowing the exact amount of a feature that is actually cyclized, the results of any binding assay could be over- or under-interpreted. In some embodiments, binding to a linear peptide is more beneficial than binding to a constrained peptide. In other embodiments, binding to constrained peptides is preferred. In either case, knowing the amount of constrained peptides in a given peptide array allows for more rigorous interpretation of the results. Disclosed herein are certain technologies to determine the amount of cyclized molecules in a given peptide feature on the array. Only peptide features that are resistant to cyclization are detected, which, when the cyclization efficiency is high, is advantageous for identifying those peptide features with low cyclization efficiency.

In some embodiments, the technologies include in situ mass spectrometry of synthesized peptides directly from the silicon wafer. Mass spectrometry is performed by incorporating a gas-phase cleavable linker between the silicon surface and the synthesized peptides so that cleavage of the peptide is done without diffusion from the array feature. Following peptide cleavage, Matrix-Assisted Laser Desorption Ionization (MALDI) mass spectrometry is performed directly on the silicon surface by applying a thin aerosol matrix layer and subsequently focusing the MALDI laser on individual peptide features to acquire a mass spectrum for each synthesized peptide. In some embodiments, the cyclized and acyclic peptides have different molecular weights and can be identified by their MALDI spectra. In some embodiments, the cyclized and acyclic peptides have the same molecular weight, and a quenching step can be used on one of the functional groups of the acyclic precursor in order to identify them by their now different molecular weights as observed in the MALDI spectra. Mass spectrometers do not actually measure the molecular weight (MW) of a compound, but rather the mass-to-charge ratio (m/z) of an ionic form of a molecule, either positively charged or negatively charged. In MALDI spectra, most often singly charged ions, either +1 charge state or −1 charge state are observed. When the source of the ion is a fixed (non-titratable) charge, for example a quaternary ammonium ion or a quaternary phosphonium ion, then the measured m/z equals the molecular weight of the ion. When the charge source of a positive ion is a proton, then the measured m/z equals the molecular weight plus the mass of that proton (+1.00). When the charge source of a positive ion is a sodium ion, then the m/z equals the MW of the ion plus the mass of the sodium ion (+22.99). Knowing the source of the charge enables the practitioner to convert between m/z and MW quantities. Different molecules have different propensities to form ions. However, usually molecules that are structurally similar, for example a peptide azide and its quenched form, will behave similarly in a MALDI experiment, and their relative peak intensities qualitatively reflect the true relative amounts within in a sample. Thus, MALDI spectra can be used to estimate the relative abundance of two or more species within a sample, provided they are structurally similar. When exact quantitation is required, isotopically labeled versions of the molecule can be synthesized as standards and a known amount spiked into the sample of interest. The analyte and its heavy isotope version behave identically in the MALDI experiment enabling true quantitation by measurement of relative m/z peak intensities.

In some embodiments, the technologies include a fluorescence-based assay for monitoring peptide cyclization. In some embodiments, the uncyclized fraction of peptides is labeled with an affinity handle or a reporter probe (FIG. 4). In some embodiments, the uncyclized fraction of peptides is labeled with an affinity handle which is biotin, E-tag, FLAG-tag, His-tag, V5 epitope tag, HA tag and the like. In some embodiments, the uncyclized fraction of peptides is labeled with an affinity handle which is biotin. In such embodiments, the affinity handle is subsequently contacted with a complementary molecule containing a reporter construct. In some embodiments, the uncyclized fraction of peptides is labeled with an affinity handle which is biotin, and the reporter is a fluorescent-streptavidin. In other embodiments, the uncyclized fraction of peptides is labeled with a reporter probe directly, such as Cy3-DBCO with an unreacted azide, or Cy3-NHS ester with an unreacted amine. The fluorescence-based assay monitors the degree of peptide cyclization across the array by detecting the uncyclized form of the peptides. In some embodiments, the fluorescence is subsequently correlated to MALDI spectra data to give an estimate of cyclization efficiency for each position. As shown in the examples, some cyclization systems have excellent cyclization efficiencies (for example, >90% of sequences cyclize with >90% yield) while others have moderate efficiency (for example >50% sequences cyclize with >80% yield). The whole array fluorescent assay provides useful information on the cyclization extent of each peptide feature on the array. This can in turn be used to subtract questionable data points from a data set thus increasing the confidence of the analysis and the power of the experiment. In some aspects, secondary antibody (detector/AF555-conjugate) alone with no primary antibody or serum sample is included as a negative control.

Technologies disclosed herein, thereby provide for methods to detect the amount of cyclic peptides in a given peptide feature on an array. This allows for selected analysis of which peptide features to include in the analysis of screening the array.

In another aspect, disclosed herein is a method for determining which peptide features of a peptide array have successfully cyclized after a cyclization step, and the % to which they have successfully cyclized, using MALDI mass spectrometry.

In another aspect, disclosed herein is a method for determining which peptide features of a cyclic peptide microarray have successfully cyclized after a cyclization step, and the % to which they have successfully cyclized, the method comprising:
(a) labeling the uncyclized fraction of the cyclic peptide feature with an affinity handle or a reporter probe;
(b) detecting the uncyclized fraction.

In some embodiments, said labeling occurs through a free amine of the uncyclized fraction. In some embodiments, the labeling occurs by reacting an NHS ester with the free amine. In some embodiments, the NHS ester comprises a fluorescent dye. In some embodiments, the fluorescent dye is an acridine dye, an Alexa Fluor™ dye, a BODIPY dye, a cyanine dye, a fluorone dye, an oxazine dye, a phenanthridine dye, or a rhodamine dye. In some embodiments, the fluorescent dye is an Alexa Fluor™ dye or a cyanine dye. In some embodiments, the fluorescent dye is Alexa Fluor™ 350, Alexa Fluor™ 405, Alexa Fluor™ 430, Alexa Fluor™ 488, Alexa Fluor™ 514, Alexa Fluor™ 532, Alexa Fluor™ 546, Alexa Fluor™ 555, Alexa Fluor™ 568, Alexa Fluor™ 594, Alexa Fluor™ 633, Alexa Fluor™ 635, Alexa Fluor™ 647, Alexa Fluor™ 660, Alexa Fluor™ 680, Alexa Fluor™ 700, Alexa Fluor™ 750, Alexa Fluor™ 790, Cy2, Cy3, Cy3B, Cy3.5, Cy5, Cy5.5, or Cy7.

In some embodiments, the NHS ester comprises biotin. In some embodiments, the method further comprises the step of adding a streptavidin-bound fluorescent dye; after step (a) and before step (b). In some embodiments, the fluorescent dye is an acridine dye, Alexa Fluor™dye, a BODIPY dye, a cyanine dye, a fluorone dye, an oxazine dye, a phenanthridine dye, or a rhodamine dye. In some embodiments, the fluorescent dye is an Alexa Fluor™ dye or a cyanine dye. In some embodiments, the fluorescent dye is Alexa Fluor™ 350, Alexa Fluor™ 405, Alexa Fluor™ 430, Alexa Fluor™ 488, Alexa Fluor™ 514, Alexa Fluor™ 532, Alexa Fluor™ 546, Alexa Fluor™ 555, Alexa Fluor™ 568, Alexa Fluor™ 594, Alexa Fluor™ 633, Alexa Fluor™ 635, Alexa Fluor™ 647, Alexa Fluor™ 660, Alexa Fluor™ 680, Alexa Fluor™ 700, Alexa Fluor™ 750, Alexa Fluor™ 790, Cy2, Cy3, Cy3B, Cy3.5, Cy5, Cy5.5, or Cy7.

In some embodiments, said labeling occurs through an azide of the uncyclized fraction.

In some embodiments, wherein said labeling occurs by reacting a diarylcyclooctyne (DBCO) derivative with the azide. In some embodiments, the DBCO derivative is a diarylazocyclooctyne derivative. In some embodiments, the DBCO derivative comprises a fluorescent dye. In some embodiments, the fluorescent dye is an acridine dye, an Alexa Fluor™ dye, a BODIPY dye, a cyanine dye, a fluorone dye, an oxazine dye, a phenanthridine dye, or a rhodamine dye. In some embodiments, the fluorescent dye is an Alexa Fluor™ dye or a cyanine dye. In some embodiments, the fluorescent dye is Alexa Fluor™ 350, Alexa Fluor™ 405, Alexa Fluor™ 430, Alexa Fluor™ 488, Alexa Fluor™ 514, Alexa Fluor™ 532, Alexa Fluor™ 546, Alexa Fluor™ 555, Alexa Fluor™ 568, Alexa Fluor™ 594, Alexa Fluor™ 633, Alexa Fluor™ 635, Alexa Fluor™ 647, Alexa Fluor™ 660, Alexa Fluor™ 680, Alexa Fluor™ 700, Alexa Fluor™ 750, Alexa Fluor™ 790, Cy2, Cy3, Cy3B, Cy3.5, Cy5, Cy5.5, or Cy7.

### Use of the Arrays for Binding Characterization

In various embodiments, the methods, systems and technologies disclosed herein provide peptide array platforms for detecting binding events, including protein-to-protein, protein-to-peptide and peptide-to-peptide binding occurring on the peptide arrays. In some embodiments, the arrays comprise individual peptides within a feature on the array spaced less than 0.5 nm, less than 1 nm, less than 2 nm, less than 3 nm, less than 4 nm, less than 5 nm, less than 6 nm, less than 7 nm, less than 8 nm, less than 9 nm, less than 10 nm apart, less than 11 nm apart, less than 12 nm apart, less than 13 nm apart, less than 14 nm part, less than 15 nm apart, less than 16 nm apart, less than 17 nm apart, less than 18 nm apart, less than 19 nm apart, or less than 20 nm apart.

In some embodiments, the methods, systems and technologies disclosed herein provide peptide array platforms for characterizing binding events, including protein-to-protein, protein-to-peptide and peptide-to-peptide binding occurring on the peptide arrays. In some embodiments, binding characteristics measured include affinity or avidity or other measurement of a protein or peptide binding to a binding partner on the surface of the peptide array.

In some embodiments, the methods, systems and technologies disclosed herein provide constrained lead peptides that can be developed into useful molecules for antagonizing a protein-protein interaction, for example cytokine binding to a cell surface receptor. Examples from the scientific literature include the development of cyclic peptide IL-5 receptor antagonists (England et al, Proc Natl Acad Sci USA. 2000 Jun. 6; 97(12): 6862-6867.) and cyclic peptide integrin antagonists (Wang et al. Mol Cancer Ther. 2016 February; 15(2): 232-240.).

In some embodiments, the methods, systems and technologies disclosed herein provide constrained lead peptides that can be developed into useful molecules that act as protein agonist mimetics, for example cyclic peptide agonists of the erythropoietin receptor. (Wrighton et al. Science. 1996 Jul. 26; 273(5274):458-64).

In some embodiments, the methods, systems and technologies disclosed herein provide constrained lead peptides that are derived from naturally occurring linear peptide precursors, for example peptide hormones. The constrained versions will have useful properties that differ from the parent linear hormone. These include receptor subtype selectivity(s), enhanced agonist activity, partial agonist activity, or antagonist activity. For example, cyclic peptides derived from naturally occurring analgesic peptide enkephalins show enhanced potency and opioid receptor subtype selectivities (Remesic et al. Curr Med Chem. 2016; 23(13):1288-303.). In addition to their desirable pharmacological properties, constrained peptides can also provide other drug-like properties relative to their linear precursors. These include reduced metabolic clearance rates by exoproteases and endoproteases, increased exposure to intracellular drug targets, and increased penetration of the blood-brain barrier.

In yet other embodiments, the methods, systems and technologies disclosed herein provide characterization of protein-to-protein, protein-to-peptide and peptide-to-peptide binding occurring on the peptide arrays, and ways of improving protein-to-protein, protein-to-peptide and peptide-to-peptide binding occurring on the peptide arrays. In some embodiments, the methods, systems and technologies disclosed herein allow stratification of binding events, including the identification of lower affinity binding partners to a target protein or peptide. In some embodiments, relative binding strengths can be measured, and peptides that bind to the target protein or peptide can be identified and characterized with regards to off-target sequences that can be detected when in a three-dimensional constrained structure.

In one aspect, disclosed herein is a method for characterizing protein binding to peptide targets, the method comprising:
(a) synthesizing a peptide array comprising at least one cyclic peptide feature which comprises peptides of Formula (I):

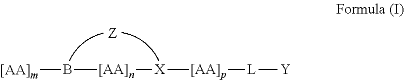

wherein,
each AA is independently a natural or unnatural amino acid residue;
m, n, and p are each independently an integer from 0 to 100;
X is a natural or unnatural amino acid residue;
B is an acid residue; wherein when m is not 0, B is a natural or unnatural amino acid residue;
   wherein each AA, B, and X residue is connected to adjacent residues through peptide bonds;
Z is a linker that connects residues X and B;
L is a tether that is optionally present; and
Y is a point of connection connecting the tether to a solid support having a reactive surface;
(b) contacting said peptide array with said protein at one or more concentrations in the presence and absence of a plurality of competitor molecules at one or more concentrations to obtain one or more individual peptide features, wherein the identified one or more individual peptide features exhibit a binding signal measured in the presence of the plurality of competitor molecules at one or more concentrations within a predetermined threshold of the binding signal measured in the absence of the plurality of competitor peptides; and
(c) characterizing binding of the protein against the peptide features on the peptide array.

In some embodiments, the peptide array is synthesized in situ.

In some embodiments, the method further comprises the steps:
(d) determining which peptide features of said peptide array have successfully cyclized after a cyclization step, and the % to which they have successfully cyclized;
(e) identifying peptide features from the array that have not cyclized to a second predetermined threshold; and
(f) removing peptide features in step (e) from the characterization in step (c).

In some embodiments, the second predetermined threshold of step (e) is at least 50%, at least 60%, at least 70%, at least 75%, at least 80%, at least 85%, at least 90%, or at least 95%.

In some embodiments, the predetermined threshold in step (b) is a binding signal in the presence of competitor molecules within at least 20-fold of the binding signal in the absence of competitor peptides.

In some embodiments, the predetermined threshold in step (b) is a binding signal in the presence of competitor molecules of at least 5% of the binding signal as compared in the absence of competitor.

In some embodiments, said protein is an antibody, a receptor, a receptor ligand, an enzyme, or a protein involved in another peptide-protein interaction.

In some embodiments, said competitor molecules comprise peptides. In some embodiments, the competitor molecules comprise a biological sample. In some embodiments, the biological sample is derived from donor blood products (serum, plasma, blood cells, platelets), tissue samples (donor or tissue culture cell lines), pathogen preparations (intact or lysate), purified antigens (protein or carbohydrate), or purified antibodies (monoclonal or polyclonal, different species, or differently labeled).

In some embodiments, the peptide array comprises at least 1000 unique peptide features. In some embodiments, the peptide array comprises at least 10,000 unique peptide features. In some embodiments, the peptide array comprises at least 100,000 unique peptide features. In some embodiments, wherein the peptide array comprises at least 1,000,000 unique peptide features.

In some embodiments, the binding signal is measured as an intensity of the signal in the absence and presence of the competitor peptides at one or more concentrations. In some embodiments, an apparent K_{d} is obtained in the presence and absence of the competitor peptides at one or more concentrations.

In another aspect, disclosed herein is a method for characterizing protein binding to peptide targets, the method comprising:
(a) synthesizing a peptide array comprising at least one cyclic peptide feature which comprises peptides of Formula (I):

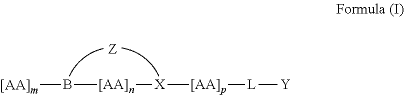

wherein,
each AA is independently a natural or unnatural amino acid residue;
m, n, and p are each independently an integer from 0 to 100;
X is a natural or unnatural amino acid residue;
B is an acid residue; wherein when m is not 0, B is a natural or unnatural amino acid residue;
   wherein each AA, B, and X residue is connected to adjacent residues through peptide bonds;
Z is a linker that connects residues X and B;
L is a tether that is optionally present; and
Y is a point of connection connecting the tether to a solid support having a reactive surface;
(b) contacting said peptide array with said protein at one or more concentrations to obtain one or more individual peptide features, wherein the identified one or more individual peptide features exhibit a binding signal measured at one or more concentrations; and
(c) characterizing binding of the protein against the peptide features on the peptide array.

In some embodiments, the peptide array is synthesized in situ.

In some embodiments, the method further comprises the steps:
(d) determining which peptide features of said peptide array have successfully cyclized after a cyclization step, and the % to which they have successfully cyclized;
(e) identifying peptide features from the array that have not cyclized to a predetermined threshold; and
(f) removing peptide features in step (e) from the characterization in step (c).

In some embodiments, the predetermined threshold of step (e) is at least 50%, at least 60%, at least 70%, at least 75%, at least 80%, at least 85%, at least 90%, or at least 95%.

In some embodiments, said protein is an antibody, a receptor, a receptor ligand, an enzyme, or a protein involved in another protein-protein interaction.

In some embodiments, the peptide array comprises at least 1000 unique peptide features. In some embodiments, the peptide array comprises at least 10,000 unique peptide features. In some embodiments, the peptide array comprises at least 100,000 unique peptide features. In some embodiments, the peptide array comprises at least 1,000,000 unique peptide features.

In some embodiments, the binding signal is measured as an intensity of the signal.

### Testing and Samples

Biological samples are added and allowed to incubate with the peptide arrays. Biological samples include blood, dried blood, serum, plasma, saliva, tears, tear duct fluid, cheek swab, biopsy, tissue, skin, hair, cerebrospinal fluid sample, feces, or urine sample. In some embodiments, a subject can, for example, use a “fingerstick”, or “fingerprick” to draw a small quantity of blood and add it to a surface, such as a filter paper or other absorbent source, or in a vial or container and optionally dried. A biological sample provided by a subject can be concentrated or dilute. In yet other embodiments, a biological sample is a purified antibody preparation, including a monoclonal antibody, a polyclonal antibody, an antibody fragment, single chain antibodies, chimeric antibodies, humanized antibodies, an antibody drug conjugate or the like. In yet other embodiments, a biological sample is a cell culture or other growth medium used to propagate recombinant antibodies in cell hosts.

In some embodiments, no more than about 0.5 nl to about 50 μl of biological sample is required for analysis by a method or system as disclosed herein. In yet other embodiments, about 0.5 nl to 25 μl, about 5 nl to 10 μl, about 5 nl to 5 μl, about 10 nl to 5 μl, about 10 nl to 2.5 μl, about 100 nl to 2.5 μl, or about 100 nl to 1 μl of biological sample is required for analysis. In some embodiments, a subject can provide a solid biological sample, from for example, a biopsy or a tissue. In some embodiments, about 0.1 mg, about 0.5 mg, about 1 mg, about 5 mgs, about 10 mgs, about 15 mgs, about 20 mgs, about 25 mgs, about 30 mgs, about 35 mgs, about 40 mgs, about 45 mgs, about 50 mgs, about 55 mgs, about 60 mgs, about 65 mgs, about 7 mgs, about 75 mgs, about 80 mgs, about 85 mgs, about 90 mgs, about 95 mgs, or about 100 mgs of biological sample are required for analysis by a method or system as disclosed herein.

In some embodiments, biological samples from a subject are too concentrated and require a dilution prior to being contacted with an array of the invention. A plurality of dilutions can be applied to a biological sample prior to contacting the sample with an array of the invention. A dilution can be a serial dilution, which can result in a geometric progression of the concentration in a logarithmic fashion. For example, a ten-fold serial dilution can be 1 M, 0.01 M, 0.001 M, and a geometric progression thereof. A dilution can be, for example, a one-fold dilution, a two-fold dilution, a three-fold dilution, a four-fold dilution, a five-fold dilution, a six-fold dilution, a seven-fold dilution, an eight-fold dilution, a nine-fold dilution, a ten-fold dilution, a sixteen-fold dilution, a twenty-five-fold dilution, a thirty-two-fold dilution, a sixty-four-fold dilution, and/or a one-hundred-and-twenty-five-fold dilution.

### Detection of Binding Events

Binding interactions between components of a sample and a peptide array can be detected in a variety of formats. In some formats, components of the samples are labeled. The label can be a radioisotype or dye among others. The label can be supplied either by administering the label to a patient before obtaining a sample or by linking the label to the sample or selective component(s) thereof.

Binding interactions can also be detected using a secondary detection reagent, such as an antibody. For example, binding of antibodies in a sample to an array can be detected using a secondary antibody specific for the isotype of an antibody (e.g., IgG (including any of the subtypes, such as IgG1, IgG2, IgG3 and IgG4), IgA, IgM, IgD, IgE, IgY), or a specific substructure of an antibody (e.g., Kappa or Lambda light chains). The secondary antibody is usually labeled and can bind to all antibodies, or parts thereof, in the sample being analyzed of a particular isotype(s), for example, an antibody that binds IgA or IgA and IgG isotypes. Different secondary antibodies (for example, from different hosts) can be used having different isotype specificities.

Binding interactions can also be detected using label-free methods, such as surface plasmon resonance (SPR) and mass spectrometry. SPR can provide a measure of dissociation constants, and dissociation rates, for example, using the A-100 Biocore/GE instrument for this type of analysis.

Detection of binding events can also occur in the presence of competitor peptides. In some embodiments, the competitive inhibitor is a peptide identical to, similar to or derived from a determined epitope or motif as disclosed herein. In some embodiments, the competitive inhibitor peptides comprises a mixture of at least 2, at least 3, at least 5, at least 10, at least 15, at least 20, at least 25, at least 30, at least 35, at least 40, at least 45 or at least 50 different peptides. In some embodiments, the competitor peptides comprise natural and/or non-natural amino acids. In some embodiments, the competitive inhibitor peptide comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% and/or at least 99% identical to a determined epitope or motif. In other embodiments, the competitive inhibitor peptide comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% and/or at least 99% similar to a determined epitope or motif. In some embodiments, the similarity can be determined by sequence or by structure. In other embodiments, the competitive inhibitor peptide may comprise a mixture of random or semi-random peptides. In yet other embodiments, the competitive peptide mixture can include a biological source, for example, serum, plasma or blood, added to or in place of the competitive inhibitor peptides disclosed herein. By adding competitive inhibitor peptides to the binding reaction, and measuring a change in binding signal in the absence and presence of the competitive inhibitor peptides, a measurement of specificity may be obtained that conveys information regarding the stringency of the interaction between peptides on the array and the biological sample. Specificity can be measured in terms of the affinity (Kd) measured in the presence of competitor and/or the number of identified peptides with a determined motif or sequence that bind to the biological sample or antibody and identified as a putative binding site.

Binding interactions can also occur in the presence of a chaotropic agent. A chaotropic agent can be potassium chloride, potassium thiocyanate, sodium thiocyanate, ammonium sulfate, guanidinium choloride, lithium percholorate, lithium acetate, magnesium choride, sodium dodecyl sulfate, thiourea, urea, n-butanol, ethanol, phenol, 2-propanol and the like. In some embodiments, the chaotropic salt is at least 0.01M at least 0.1M, at least 0.5M, at least 1.0 M, at least 1.5 M and at least 3.0 M. In some embodiments, the chaotropic salt is added to the incubation buffer. In yet other embodiments, the chaotropic agent is applied in the wash buffer following the primary sample addition, yet before the secondary (detector) antibody. In some embodiments, specificity is measured in terms of the avidity reflected in the concentration of chaotropic agent used to release the sample from the array. In some embodiments, the sample is a purified antibody, biological sample, purified protein, cell culture, or the like.

### Uses of the Peptide Arrays

In some embodiments, detection of protein binding on a peptide array poses some challenges that can be addressed by the technologies disclosed herein. The technologies disclosed herein allow for the in situ synthesis of constrained cyclic peptides on an array. The constrained cyclic peptide arrays can be used to probe any protein-peptide interaction, including but not limited to detection and characterization of protein-peptide, protein-protein and/or peptide-peptide interactions to determine characteristics of the binding example, for example, affinity, avidity and/or specificity of binding, as well the determination of specific binding interactions for identifying, for example, disease states or conditions in a subject. In some embodiments, the method, systems and technologies disclosed herein allow a user to determine binding characteristics between proteins or peptides to the constrained cyclic peptides on arrays as described herein. In other instances, the methods, systems and technologies disclosed herein allow a user to compare, for example, binding characteristics between proteins or peptides to both constrained cyclic peptides on arrays as well as linear peptides also bound to the arrays. The methods, systems and technologies disclosed herein provide flexibility to a user to design and implement different uses where constrained cyclic peptides can enhance or promote binding activity between proteins and/or peptides. The examples described below are exemplary of uses for the methods, systems and technologies disclosed herein, and are not limiting of the same.

### Antibody Epitope Binding Profiles

In one aspect, the constrained cyclic peptide arrays disclosed herein act as antigen screening arrays to produce antibody binding profiles on arrays that approach or correlate with solution-phase antibody binding. In some embodiments, peptide features represent the known epitope of an antibody and the surrounding sequences. In some embodiments, the array is then probed with the antibody to produce an antibody binding profile.

In some embodiments, the technologies disclosed herein address the method of antibody labeling in detection of antibody binding profiles using arrays. Direct fluorescence labeling of antibodies frequently suppresses, modifies or abrogates binding to known epitopes. To address this, the technologies disclosed herein include an indirect detection method, similar to the indirect ELISA assay, that first binds the unlabeled primary antibody (the antibody being profiled) to the array, which is followed by binding of a fluorescently labeled secondary antibody that binds to a fixed epitope on the unlabeled primary antibody (e.g. the Fc region of IgG antibodies). The binding of the labeled secondary to the primary antibody is validated prior to incubation on the arrays to ensure that the labeled secondary binds the primary antibody as expected.

In some embodiments, the methods, systems and technologies disclosed herein may be used to identify and characterize binding partners of, for example, antibodies. For example, nearly all therapeutic antibody screens incorporate some level of epitope mapping and epitope binning on a select number of leads for moving forward into a therapeutic development pipeline. Epitope binning studies map the epitopes of several lead antibodies and then bin the antibodies by their binding affinity/kinetics towards identified epitopes. The methods, systems and technologies disclosed herein may be used to screen, identify and characterize target (e.g. epitopes) and off-target binding partners of potential therapeutic antibodies by providing constrained peptide arrays that could detect binding events with higher sensitivity.

### Receptor Ligand Binding Profiles

In another aspect, the constrained cyclic peptide arrays disclosed herein act as screening arrays for receptor-ligand interactions. In some embodiments, the receptor-ligand interaction occurs at a known recognition site on the receptor. In some embodiments, the array represents the known contact point on the receptor and the surrounding sequences. In some embodiments, the array represents the known ligand for the receptor. In some embodiments, the array is then probed with either the receptor, ligand or portions thereof to produce a ligand binding profile.

### Receptor Agonist and Antagonist Binding Profiles

In another aspect, the constrained cyclic peptide arrays disclosed herein can be used to identify new ligands for a known receptor. In such embodiments, the array is made of random, or semi-random peptide features. In some embodiments, the array is then probed with a soluble form of the receptor or a known receptor binding site. In some embodiments, new ligands are identified with the resulting receptor binding profile. In some embodiments, the newly identified ligand is an antagonist, or an agonist of the receptor.

### Enzyme Substrate Binding Profile

In another aspect, the constrained cyclic peptide arrays disclosed herein can be used to identify substrates for a known enzyme. In some embodiments, the array is made of random or semi-random peptide features that may act as the substrate for the enzyme. In some embodiments, the array is probed with an enzyme. In some embodiments, the peptides which are changed by the enzyme are identified to produce a substrate binding profile. For example, the array may be made up of peptide features comprising free hydroxyl containing amino acids and the array can be probed with a kinase, wherein new phosphoryl groups can be detected on the array by, for example, anti-phospho-amino-acid antibodies. In another example, the array can be probed with a protease and the subsequent linear peptide features can be identified, for example, by the methods disclosed herein.

In a further aspect, enzyme inhibitors can be identified using the constrained cyclic peptide arrays disclosed herein. In some embodiments, the array is made of random or semi-random peptide features that may act as the substrate for the enzyme. In some embodiments, the array is probed with an enzyme in the presence and absence of different concentrations of a competitor or inhibitor molecules. In some embodiments, the differences in the rates of enzyme modification of array features in the presence and absence of different concentrations of competitor or inhibitor molecules are then used to identify competitor or inhibitor molecules.

In another aspect, protease inhibitor molecules are identified by probing an array with a fluorescently labeled protease. In some embodiment, constrained peptides that are substrates will be cleaved and the protease is unlikely to bind the product. In some embodiments, constrained peptides that are inhibitors will bind the protease and will be identified as fluorescent features.

### Protein-Protein Interaction Inhibitors

In another aspect, protein-protein interaction inhibitors can be identified using the constrained cyclic peptide arrays disclosed herein. In some embodiments, features of the array represent sequences one protein involved in a protein-protein interaction. In some embodiments, the array is probed with a soluble form of a second protein involved in the protein-protein interaction. In some embodiments, a binding profile is produced corresponding to the protein-protein interaction. In further embodiments, the array is probed with a soluble form of a second protein involved in the protein-protein interaction in the absence and presence of different concentrations of a competitor or inhibitor molecules. In some embodiments, the differences in the protein-protein binding profiles in the presence and absence of different concentrations of competitor or inhibitor molecules are then used to identify competitor or inhibitor molecules.

### Detection of Binding Partners to Protein Libraries

In some aspects, the methods, systems and technologies described herein may be used to detect and identify putative binding partners to known or unknown proteins, target molecules or portions thereof. In one embodiment, the constrained peptide libraries disclosed in the methods, systems and technologies described herein may comprise known or unknown libraries, for example, phage libraries that contain a plurality of peptides or peptide congeners representing or including, for example, expressed proteins in specific protein classes and diseases states (e.g., libraries comprising human scFvs, or proteins expressed in specific cancers), as well as libraries representing or including, for example, entire proteomes (e.g., mouse or human proteome).

In some embodiments, the libraries comprising a plurality of peptides or peptide congeners may be converted to constrained peptides and anchored onto a peptide array using the methods, systems and technologies disclosed herein. In some embodiments, the library constrained peptide arrays may be probed with a known protein, or biological sample from a subject with a known disease, to detect and identify putative binding partners to the known protein or proteins expressed in the subject using the methods, systems and technologies disclosed herein. In some embodiments, the binding characteristics between the known protein or expressed proteins in the subject biological sample may be further characterized using the same library constrained peptide array or a different constrained, linear or mixed (constrained and linear) peptide array to determine, for example, specificity, affinity or other binding characteristics.

In particular, described herein is a method to identify potential binding partners to a protein of interest. In some embodiments, two peptide arrays with identical feature sequences, wherein one array is comprises linear peptide features and the other array comprises cyclic peptide features, are used to identify peptide sequences which bind the protein of interest. In some embodiments, binding to the peptide arrays is noticeably different depending on if the peptides are linear or constrained. In some embodiments, the sequences that preferentially bind the constrained features are identified. In some embodiments, the key binding residues are identified through sequence alignment. In some embodiments, peptides that preferentially bind the cyclic features show relatively conserved binding sequences while peptides that preferentially bind the linear features do not. In some embodiments, the cyclic and linear arrays are counter screened with other proteins to identify peptide features that do not specifically bind to the protein of interest. In some embodiments, the arrays are counter screened with immunoglobulin depleted human serum (Non-Immunoglobulin Serum Components or NISC) to identify generally sticky peptide features. In some embodiments, the protein of interest is diaphorase. In some embodiments, the arrays are counter screened with Ferredoxin NADP+ Reductase (FNR) or Ferredoxin.

In some embodiments, binding of peptide features to a protein of interest is confirmed through independent synthesis of the identified features. In some embodiments, sequences which preferentially bind can be synthesized in both linear and constrained form. In some embodiments, binding to the protein of interest is then confirmed, for example, through affinity capture methods. For example, by linking a biotinyl group to the synthesized peptide, the protein of interest can be captured on a streptavidin plate or chip.

### Use of the Arrays for Detecting or Characterizing Diseases or Conditions

In some embodiments, the peptide arrays disclosed herein may be used to detect and/or characterize disease states or conditions, such as autoimmune diseases, inflammatory diseases, metabolic disorders, infectious diseases, cancer or tumors and the like.

In some instances, the peptide arrays disclosed herein are used to detect and/or characterize disease state[s], such as autoimmune diseases or conditions. In some embodiments, the autoimmune disease is arthritis (such as rheumatoid arthritis, psoriatic arthritis or osteoarthritis); transplant (such as organ transplant, acute transplant or heterograft or homograft (such as is employed in burn treatment)) rejection; protection from ischemic or reperfusion injury such as ischemic or reperfusion injury incurred during organ transplantation, myocardial infarction, stroke or other causes; transplantation tolerance induction; multiple sclerosis; inflammatory bowel disease, including ulcerative colitis and Crohn's disease; lupus (systemic lupus erythematosus); graft vs. host diseases; T-cell mediated hypersensitivity diseases, including contact hypersensitivity, delayed-type hypersensitivity, and Celiac disease; Type 1 diabetes; psoriasis; contact dermatitis; Hashimoto's thyroiditis; Sjogren's syndrome; Graves' Disease; Addison's disease; polyglandular disease; alopecia; pernicious anemia; vitiligo; Guillain-Barre syndrome; glomerulonephritis and uticaria.

In some instances, the diseases or conditions include infectious diseases, including exemplary bacterial, fungal and viral diseases, such as Valley Fever, Q-fever, Tularemia (Francisella tularensis), Rickettsia rickettsii, HSV types I and II, HVB, HVC, CMV, Epstein Barr virus, JC virus, influenza, A, B or C, adenovirus, and HIV.

In some instances, the diseases or conditions include inflammatory and/or allergic diseases such as respiratory allergies (asthma, hayfever, allergic rhinitis) or skin allergies; acute inflammatory responses and the like. In some instances, the diseases or conditions include inflammation or inflammatory diseases or conditions, including chronic diseases, such as inflammatory or allergic diseases such as systemic anaphylaxis or hypersensitivity responses and other diseases in which inflammatory responses are to be inhibited. In yet other instances, the diseases or conditions are metabolic disorders that are sensitive to inhibition of TNF or IL-1 signaling, such as obesity and complications thereof, type II diabetes, Syndrome X, insulin resistance, hyperglycemia, hyperuricemia, hyperinsulinemia, cachexia, hypercholesterolemia, hyperlipidemia, dyslipidemia, mixed dyslipidemia, hypertriglyceridemia and eating disorders such as anorexia nervosa and bulimia. In still other instances, the disease is an infectious disease, e.g., septic shock and bacteremia. In yet other embodiments, the disease or condition is a cardiovascular disorder, such as acute heart failure, hypotension, hypertension, angina pectoris, myocardial infarction, cardiomyopathy, congestive heart failure, atherosclerosis, coronary artery disease, restenosis and vascular stenosis.

In some instances, the peptide arrays disclosed herein are used to detect and/or characterize disease state[s], such as cancer. In some instances, the disease is cancer or tumors. In some embodiments, the cancer types include adrenal cortical cancer, anal cancer, aplastic anemia, bile duct cancer, bladder cancer, bone cancer, bone metastasis, CNS brain tumors, breast cancer, cervical cancer, vaginal cancer, Non-Hodgkin's lymphoma, gastrointestinal cancers, including stomach, colon and rectal cancer, ovarian cancer, endometrial cancer, esophageal cancer, Ewing's family of tumors, eye cancer, gallbladder cancer, gastrointestinal carcinoid tumors, gastrointestinal stromal tumors, Hodgkin's disease, Kaposi's sarcoma, kidney cancer, laryngeal and hypopharyngeal cancer, leukemia, liver cancer, lung cancer, multiple myeloma, myelodysplastic syndrome, nasal cavity and paranasal cancer, pancreatic cancer, penile cancer, pituitary tumor, prostate cancer, retinoblastoma, rhabdomyosarcoma, salivary gland cancer, sarcoma, skin cancer, testicular cancer.

In some instances, the peptide arrays disclosed herein are used to identify lead molecules that agonize or antagonize a protein-protein interaction, peptide-protein interaction, or peptide-peptide interaction. Many biological processes depend upon such interactions and as such they serve as useful and desirable points of intervention for many diseases, including cancer, metabolic diseases, chronic or acute pain, cardiovascular disease, and others. In some embodiments, such leads can be developed into actual therapies for treating specific diseases.

### Mass Spectrometry Detection

In some embodiments, the technologies disclosed herein utilize in situ mass spectrometry detection of a set of peptide sequences on each chip that interrogate every synthesis step to quantify the efficiency and purity of each step. The technologies build on initial MALDI development to enable yield and purity quantitation. In some embodiments, in situ MALDI mass spectra are acquired from the synthesized peptide array by incorporating a gas-phase cleavable, safety-catch linker (SCL) (see: Heidler, et al., Bioorg. Med. Chem. 2005, 13(3): 585-99) that is stable to binding assay conditions and can be cleaved without diffusion from the silicon surface using ammonia gas. The SCL will be coupled to the amine functionalized silicon surface and peptides will be built from the SCL surface linkage. After peptide array synthesis on an 8-inch wafer, 13 microscope slide dimensioned chips and several replicate analytical MALDI arrays are diced from the wafer and one or more analytical chips are reserved for MALDI mass spectra acquisition. The SCL is activated for subsequent cleavage by treatment with and alkylating agent, for example iodoactetonitrile, then ammonia gas treatment of the MALDI reserved chip cleaves the synthesized peptide from the silicon surface without diffusion. Following gas-phase cleavage, a MALDI matrix that facilitates peptide desorption/ionization is applied to the chip using microdroplet aerosol application without diffusion of the cleaved peptides on the array surface. In some embodiments, the MALDI matrix is made of α-cyano-4-hydroxycinnamic acid (CHCA), or sinapinic acid. In some embodiments, the MALDI matrix is made of α-cyano-4-hydroxycinnamic acid (CHCA). Finally, MALDI mass spectra are acquired in situ from the synthesized peptide array by aligning the MALDI laser to specific cleaved peptide array features relative to a set of alignment fiducial markers to ensure the laser is centered on the intended array feature for mass spectrum acquisition.

Alternatively, the SCL can be replaced with a diketopiperazine (DKP) precursor linker, for example a protected lysyl-prolyl-glycolic acid ester moiety (Cbz-Lys(Boc)-Pro-O-Glyc). Following incorporation of the DKP precursor onto peptide features on the analytical chip, peptide array synthesis is conducted through the Lys(Boc) side chain. Following array peptide synthesis the precursor is activated to its self-cleavable form by strong acid removal of the N-terminal Cbz group followed by washing of the substrate with acidic solvents such as 2% TFA in IPA. Upon treatment with a base, for example gaseous ammonia, the deprotected peptide ester cyclizes to a DKP with concomitant cleavage of the Pro-O-Glyc ester bond resulting in peptide detachment from the support.

In some embodiments, to quantify the efficiency of peptide synthesis steps by MALDI mass spectrometry, a set of 200-1000 μm² MALDI synthesis-analysis array features is included on the SiO₂ wafer for post synthesis analysis. The features can contain peptides of 1 to about 30 amino acids in length. Following synthesis and wafer dicing, one of more of the analytical chips is N-terminal deprotected by treatment with an acid such as TFA and washed well. The chip is then treated with a solution tris(2,4,6-trimethoxyphenyl)phosphonium succinimidyl ester (TMPP-NHS). TMPP is a MALDI signal enhancer that installs a fixed positive charge in the analyte, and aids in the comparison of relative peak intensities for purity and yield estimation.

### Binding Profile Reproducibility

In various embodiments, the technologies disclosed herein includes quantifying intra- and inter-array binding profile reproducibility with a set of 5 engineered antibodies and confirm binding profiles with peptide resynthesis and surface plasmon resonance (SPR).

In some embodiments, a set of 5 monoclonal antibodies and 5 separate arrays are used to quantify antibody binding profile reproducibility (i.e., % CV). By using a defined set of antibodies, antibody concentration and sample composition can be tightly controlled to measure variability of the array production vs. variability in the samples or assay.

In an exemplary embodiment to test the binding profile reproducibility obtained, five unrelated peptide epitopes with lengths in the range of 6-10 amino acids will be identified from literature and used as input sequences for 5 separate peptide array syntheses of epitope variant libraries. Five IgG monoclonal antibodies engineered to bind the selected epitopes are used. Each of the five antibodies is bound separately to their respective variant library array. Primary antibody binding is labeled using a fluorescently labeled anti-IgG Fc secondary antibody that binds to the Fc region of the primary IgG antibody based on an indirect assay protocol. Intra-array % CVs will be calculated using replicate peptide feature fluorescence intensities within one array. Inter-array % CVs will be calculated using identical feature fluorescence intensities on replicate arrays. Five epitope variant sequences are selected from each of the five antibody array binding profiles (25 total peptides) for synthesis and purification followed by solution-phase SPR binding analysis.

### EXAMPLES

The following non-limiting examples provide illustrative examples of the invention, but do not limit the scope of the invention.

### Example 1: Preparation of Silicon Oxide Surface for Peptide Array Synthesis

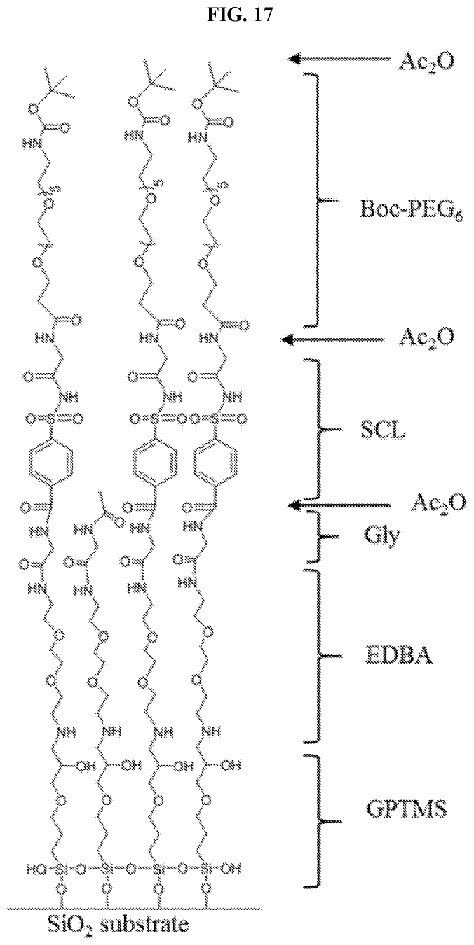

A 200 mm diameter silicon wafer with SiO₂ surface was functionalized in a multi-step process to generate the polyfunctional linker shown in FIG. 17. Briefly, the SiO₂ surface was cleaned by immersion in concentrated H₂SO₄/H₂SO₅ (Nano-Strip 2X®) at 85° C. for 30 min, and then rinsed with water and spin dried. The wafer was functionalized by treatment with 90.5 mM 3-glycidoxypropyltrimethoxysilane (GPTMS) in mesitylene at 70° C. for 95 min. The wafer was rinsed with IPA and spin dried. The epoxides were extended by vapor phase deposition of 2,2′-(ethylenedioxy)bis(ethylamine) (EDBA) in a commercial chemical vapor deposition system at 100° C. for 30 min. Reactive amines were treated with a 0.1 M solution of Boc-Gly-OH, 0.1 M HATU, 0.2 M DIEA for 30 min. Unreactive surface amines were capped by treatment with acetic anhydride in THF. The Boc group was removed by treatment of the wafer with TFA or another suitable acid followed by washing with NMP, 5% DIEA in NMP, IPA, then spin dried.

When desired, a cleavable safety catch linker (SCL) was installed by wafer treatment with 0.1 M 4-(N-((tert-butoxycarbonyl)glycyl)sulfamoyl)benzoic acid, 0.1 M HATU, 0.2 M DIEA (2×5 min) followed by washing with NMP, IPA and spin drying. The Boc group was removed by treatment of the wafer with TFA or another suitable acid followed by washing with NMP, 5% DIEA in NMP.

Finally, a long flexible linker was installed by treatment with an NMP solution of 0.1 M BocNH-PEG6-COOH, 0.1 M HATU, 0.2 M DIEA (2×5 min), and then washed with NMP. Unreactive amines were capped by treatment with Ac₂O in THF. The wafer was washed with NMP and IPA, and spin dried.

### Example 2: Synthesis of Carbobenzyloxy (Cbz) Protected Diketopiperazine Linker on Functionalized Silicon Oxide Surface

A 200 mm SiO₂ wafer with Boc-PEG6-Gly-EDBA-GPTMS surface prepared in Example 1 was subjected to a ‘dicing’ process that created SiO₂ wafer fragments of convenient dimensions, for example 20×25 mm. The fragments were placed facing upwards in glass Petri dishes (100×15 mm) and subjected to standard Boc-based solid phase peptide synthesis steps at room temperature.

Step 1) The N-terminal Boc group was removed by immersion in 12 mL of neat TFA for 10 min with gentle swirling. The TFA was decanted and the fragments washed with NMP, 5% DIEA in NMP, IPA, then dried under stream of nitrogen.

Step 2) A freshly prepared NMP solution of Fmoc-proline glycolic acid ester (Fmoc-Pro-O-Glyc-OH, CAS #131228-94-9), HATU, and DIEA (0.1, 0.1, 0.2 M respectively) was added to the fragment, which was swirled gently for 15 min. The solution was decanted and fragment washed with NMP (3×).

Step 3) The N-terminal Fmoc group was removed by immersion in 20% piperidine/NMP for 10 min. The fragment was washed with NMP (4×).

Step 4) A freshly prepared NMP solution of Cbz-Lys(Boc)-OH (CAS #2389-60-8), HATU, DIEA (0.1, 0.1, 0.2 M respectively) was added to the fragment, which was swirled gently for 15 min. The solution was decanted and fragment washed with NMP (2×), IPA (2×), then dried under nitrogen to provide the Cbz-Lys(Boc)-Pro-O-Glyc-PEG6-Gly-EDBA-GPTMS-SiO₂ surface.

### Example 3: Synthesis of Boc Protected Diketopiperazine Linker on Functionalized Silicon Oxide Surface

The procedure in Example 2 was followed except that Boc-Lys(Fmoc)-OH (CAS #115186-31-7) was used in Step 4 to provide the Boc-Lys(Fmoc)-Pro-O-Glyc-PEG6-Gly-EDBA-GPTMS-SiO₂ surface.

### Example 4: Fmoc-Based Synthesis of Click Cyclization Peptide TMPP-Lys(N3)-Phe-Pro-Gly-Leu-Pra-Leu-(Boc-Lys-Pro-O-Glyc)-PEG6-Gly-EDBA-GPTMS-SiO₂

Silicon oxide wafer fragments functionalized with the Boc protected DKP linker from Example 3 were used. A 0.20 M NMP stock solution of HATU and 0.2 M NMP stock solutions of following Nα-Fmoc protected amino acids were prepared: Fmoc-6-azido-L-norleucine (Fmoc-Lys(N3)-OH, CAS #159610-89-6), Fmoc-Phe-OH, Fmoc-Pro-OH, Fmoc-Gly-OH, Fmoc-Leu-OH, and Fmoc-propargylglycine (Fmoc-Pra-OH, CAS #198561-07-8).

The peptide was assembled stepwise by repetitive Fmoc deprotections with 20% piperidine/NMP for 10 min followed by washing with NMP (4×), then coupling of freshly prepared solution of 5 mL Fmoc amino acid stock solution, 5 mL HATU, 0.5 mL neat DIEA for 15 min, followed by washing with NMP (3×). After coupling of the last amino acid, the Fmoc group was removed and the fragment washed with NMP (2×), IPA (2×) then dried under nitrogen. A freshly prepared solution of 80 mg/mL TMPP-NHS (CAS #226409-58-1) in 1% DIEA/DMF was dripped onto the face of the wafer fragment and allowed to react at room temperature for 1.5 hr. The fragment was then washed well with NMP (2×), IPA (3×), and dried.

### Example 5: Copper(I) Catalyzed Click Cyclization of TMPP-Lys(N3)-Phe-Pro-Gly-Leu-Pra-Leu-(Boc-Lys-Pro-O-Glyc)-PEG6-Gly-EDBA-GPTMS-SiO₂

Ultra-pure water from a Milli-Q purification system (18 MΩ resistivity) was vacuum degassed just prior to use. Aqueous stock solutions of the following chemicals were freshly prepared: 7.5 mg/mL (30 mM) CuSO₄ 5H₂O, 13 mg/mL (30 mM) tris-hydroxypropyltriazolylmethylamine (THPTA, CAS #760952-88-3), 21 mg/mL (120 mM) ascorbic acid.

The TMPP labeled peptide on wafer fragment from Example 4 was placed in a screw top plastic vial. 6.0 mL of CuSO₄ 5H₂O solution and 6.0 mL of THPTA solution were mixed. The faint blue CuSO₄ solution turned to darker shade of blue. 6.0 mL of ascorbic acid solution was added and the solution turned nearly colorless. (Final concentration CuSO₄, THPTA, and ascorbic acid=10 mM, 10 mM, 40 mM, respectively). The Cu(I) solution was added to the vial containing the wafer fragment which was closed and placed in a sonication bath for 30 min. After incubation the Cu(I) solution was decanted and the fragment washed well with water (3×), then IPA (2×) and dried under nitrogen.

### Example 6: Copper-Free Conjugation of Unreacted Azidopeptide with DBCO Acid

A 4.0 mM solution of 11,12-didehydro-γ-oxo-dibenzazocine-5(6H)-butanoic acid (DBCO-acid, CAS #135016-70-2) dissolved in either methanol or 3:1 IPA/NMP was freshly prepared. A peptide wafer fragment of control azidopeptide from Example 4 and a fragment of Click cyclized peptide from Example 5 were placed in a glass Petri dish. The fragments were treated with the DBCO-acid solution with gentle swirling for 15 min, washed well with NMP (2×), IPA (2×), then dried.

### Example 7: Boc-Protected DKP Peptide Detachment and Detection by MALDI Mass Spectrometry

Peptide-wafer fragments containing the Boc protected DKP linker were prepared for MALDI analysis in the following manner:

Step 1) The Boc group was removed by treatment with neat TFA for 10 min. The TFA was decanted, the fragment washed twice with 2% TFA in IPA, then dried with nitrogen. Caution: any contact with neutral or basic liquids at this point will result in loss of peptide from the SiO₂ surface.

Step 2) Wafer fragments were placed in a stainless steel gas reaction ‘bomb’ vessel and exposed to flowing ammonia gas at room pressure for 5 min.

Step 3) A thin coating of α-cyano-4-hydroxycinnamic acid (CHCA) matrix was applied by spraying a 15 mg/mL solution of CHCA in acetonitrile/water/TFA (60:40:0.4) onto the SiO₂ surface.

Mass spectra were collected directly from the SiO₂ wafer fragment surface using a Bruker AutoFlex Speed MALDI spectrometer. FIG. 12 displays typical mass spectra obtained for the Click cyclized peptide described in Example 5 and control DBCO conjugate peptide.

### Example 8: Calculation of Click Cyclization Yield by MALDI MS

As the starting material and product of a Click cyclization yield are isomers, they cannot be measured independently in a MALDI experiment. Thus following the cyclization procedure in Example 5, unreacted azidopeptide is conjugated to DBCO acid as described in Example 6. This provides the uncyclized peptide a mass gain of 305.1 amu, differentiating it from cyclic Click product. To calculate the Click cyclization yield, the expected m/z for the monoisotopic ion for the cyclic product and linear DBCO acid adduct are calculated. The corresponding MALDI peak intensities (PI) are collected from the digital spectra and the yield is assigned based upon the formula.

% Yield=(PI product/(PI product+PI DBCO adduct))×100

### Example 9: Biotinylation of Unreacted Azidopeptide with DBCO-Biotin

Peptide microarrays on SiO₂ were biotinylated following Click cyclization reaction by treatment with a solution of DBCO-PEG₄-biotin (4-40 nM, CAS #1255942-07-4) and DBCO-acid (2 mM) in NMP for 30 min. The arrays were washed well with NMP (2×), IPA (2×), then dried under nitrogen. Arrays were then subjected to side chain deprotection as described in Example Example 10: Boc-based synthesis of head-to-tail lactam cyclization peptide Fmoc-Lys(TMPP)-Glu(OBzl)-Asn(Xan)-Phe-Ser(OBzl)-Leu-Gly-Glu(X)-OFm, X=Cbz-Lys-Pro-O-Glyc-PEG6-Gly-EDBA-GPTMS-SiO₂

Silicon oxide wafer fragments functionalized with the Cbz protected DKP linker from Example 2 were used. 0.20 M NMP stock solution of HATU and 0.20 M NMP solutions of following Nα-Boc protected amino acids were prepared: Fmoc-Lys(Boc)-OH (CAS #71989-26-9), Boc-Glu(OBzl)-OH, Boc-Asn(Xan)-OH, Boc-Phe-OH, Boc-Ser(OBzl)-OH, Boc-Leu-OH, Boc-Gly-OH, Boc-Glu-OFm (CAS #133906-29-3).

Wafer fragments were placed face up in a Petri dish and treated with neat TFA for 10 min to remove the Boc group from the Cbz-Lys(Boc)-Pro-O-Glyc cleavable linker. The TFA was decanted and the fragments washed with NMP, 5% DIEA/NMP, NMP. In the proper sequence, 5 mL of Boc amino acid was mixed with 5 mL HATU and 0.5 mL DIEA. The solution was poured into the Petri dish which was swirled gently on an orbital shaker for 15 min. The amino acid solution was decanted and the fragments washed with NMP (4×). The neat TFA Boc deprotection and amino acid coupling steps were repeated until the final Fmoc-Lys(Boc)-OH was coupled. The Boc group was then removed by treatment with neat TFA for 10 min, washed with NMP, 5% DIEA/NMP, IPA (2×), then dried under nitrogen. A freshly prepared solution of 80 mg/mL TMPP-NHS in 1% DIEA/DMF was dripped onto the face of the wafer fragments and allowed to react at room temperature for 1.5 hr. The fragment was then washed well with NMP (2×), IPA (3×), and dried.

### Example 11: Head-to-Tail Lactam Cyclization of Fmoc-Lys(TMPP)-Glu(OBzl)-Asn(Xan)-Phe-Ser(OBzl)-Leu-Gly-Glu(X)-OFm, X=Cbz-Lys-Pro-O-Glyc-PEG6-Gly-EDBA-GPTMS-SiO₂

The wafer fragment from Example 10 was placed in a Petri dish and treated with 20% piperidine/NMP for 10 min. The fragment was washed with NMP (4×). A fresh solution of 50 mM PyBOP (CAS #128625-52-5) in 1% DIEA/NMP was prepared and added to the fragments, which were swirled gently at room temperature for 1 hr. The solution was decanted and the fragments washed with NMP (2×) and IPA (2×). The cyclic lactam product can be differentiated from its linear form by mass difference of 18 amu. In order to simplify MALDI analysis, unreacted lysine amino groups can be capped by treatment with acetic anhydride in THF and pyridine. This provides an additional mass shift of 42.0 amu so that uncyclized peptide peaks can be more easily distinguished from lactam peaks.

### Example 12: Amino Acid Side Chain Deprotection and Cbz Diketopiperazine Linker Deprotection with TFA/TMS-OTf

A mixture of TFA/TMS-OTf/m-cresol/thioanisole/ethanedithiol (v:v 59:18:2.0:11:10) was carefully prepared on ice and kept at 0-5° C. prior to and during reaction. The mixture was either stirred or swirled to prevent the components from separating. SiO₂ wafer fragments were immersed in the cold mixture for 4 h then washed twice with 2% TFA in IPA then dried under nitrogen. The prepared fragments are now ready for detachment with anhydrous ammonia gas and MALDI application as described in Example 7 Steps 2 and 3.

### Example 13: Detachment of Head-to-Tail Lactam Peptide Cyclo-(Lys(TMPP)-Glu-Asn-Phe-Ser-Leu-Gly-Glu(DKP)) from SiO₂ and Detection by MALDI MS

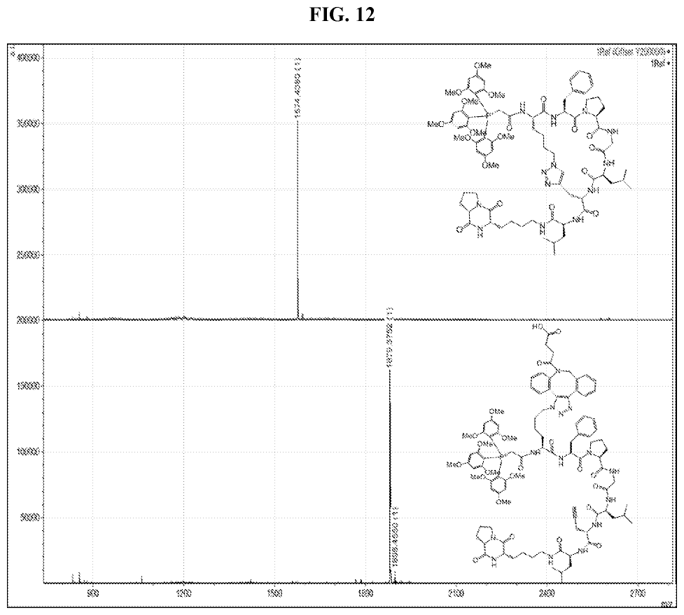

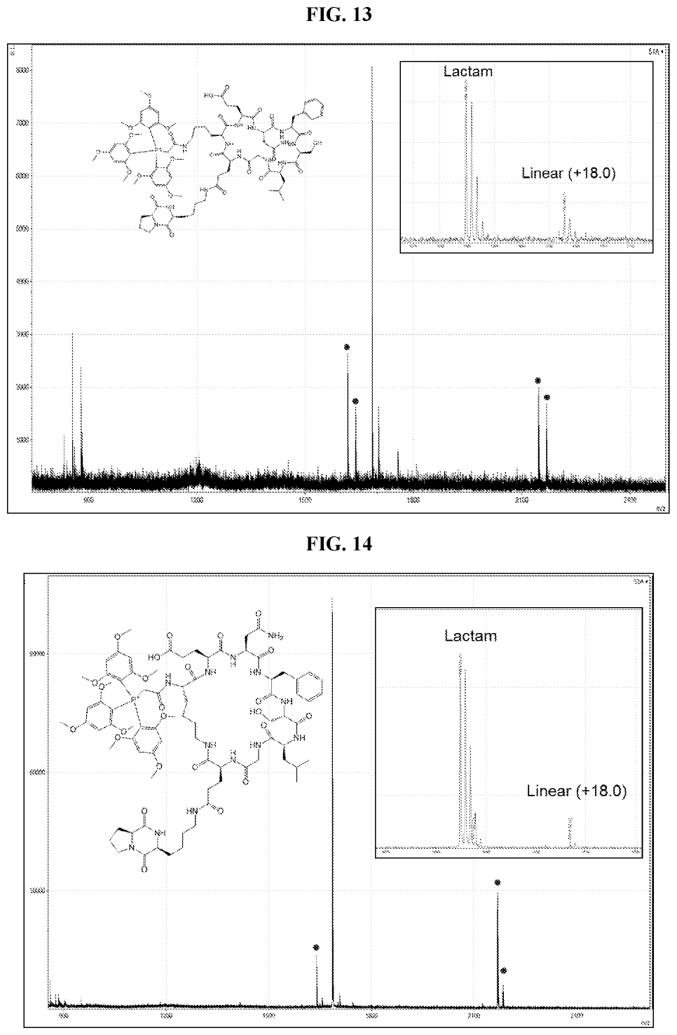

The SiO₂ wafer fragment from Example 11 was subjected to side chain deprotection as described in Example 12. The fragment was then exposed to ammonia and coated with MALDI matrix as described in Example 7. The MALDI mass spectrum is shown in FIG. 13. The product had the expected m/z peak (calc.=1684.8, found: 1684.7) and a small amount of linear peptide was detected (m/z calc.=1702.8, found: 1702.7).

### Example 14: Boc-Based Synthesis of Head-to-Sidechain Lactam Cyclization Peptide TMPP-Lys(Fmoc)-Glu(OBzl)-Asn(Xan)-Phe-Ser(OBzl)-Leu-Gly-Glu(X)-OFm, X=Cbz-Lys-Pro-O-Glyc-PEG6-Gly-EDBA-GPTMS-SiO₂

The procedure in Example 10 was followed except that Boc-Lys(Fmoc)-OH (CAS #115186-31-7) was used in place of Fmoc-Lys(Boc)-OH for the final amino acid coupling reaction.

The fragment was then processed as described in Examples 11-13, and exposed to ammonia and coated with MALDI matrix as described in Example 7. The MALDI mass spectrum is shown in FIG. 14. Calculated monoisotopic m/z for cyclized peptide=1684.8, found 1684.7).

### Example 15: Boc-Based Synthesis of Sidechain-to-Sidechain Lactam Cyclization

The procedure in Example 10 was followed except that Boc-Lys(Fmoc)-OH (CAS #115186-31-7) was used in place of Fmoc-Lys(Boc)-OH and Boc-Glu(OFm)-OH (CAS #123417-18-5) or Boc-Asp(OFm)-OH (CAS #117014-32-1) were used in place of Boc-Glu-OFm.

### Example 16: Boc-Based Synthesis of Sidechain-to-Tail Lactam Cyclization

The procedure in Example 10 was followed except that Boc-Glu(OFm)-OH (CAS #123417-18-5) or Boc-Asp(OFm)-OH (CAS #117014-32-1) were used in place of Boc-Glu-OFm.

### Example 17: Biotinylation of Unreacted Amine Following Lactam Cyclization Reaction

Peptide microarrays on SiO₂ were biotinylated following lactam cyclization reaction by treatment with a solution of NHS-C6-biotin (4-40 μM, CAS #72040-63-2) and butyric acid succinimidyl ester (2 mM, CAS #70741-39-8) in NMP for 30 min. The arrays were washed well with NMP (2×), IPA (2×), then dried under nitrogen. Arrays were then subjected to side chain deprotection as described in Example 12.

### Example 18: Determination of Cyclization Efficiency by MALDI MS

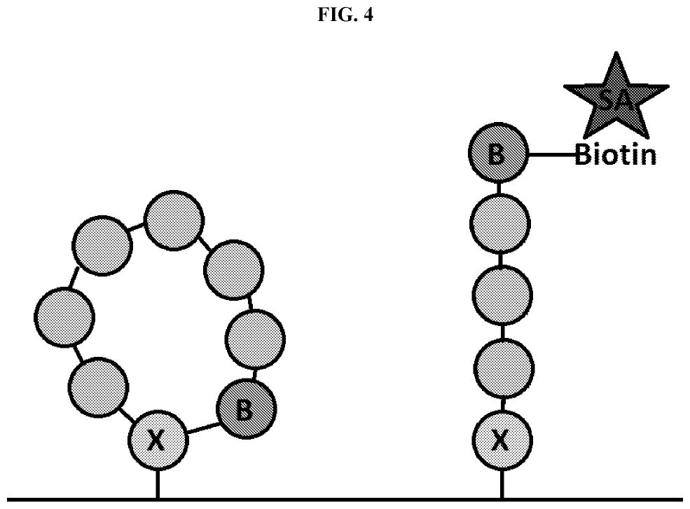

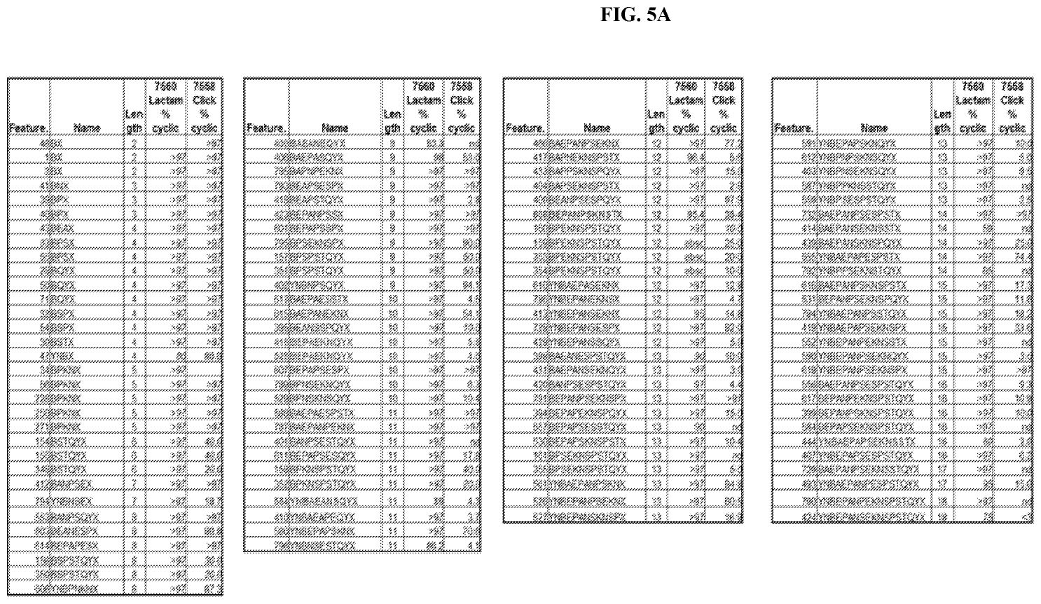

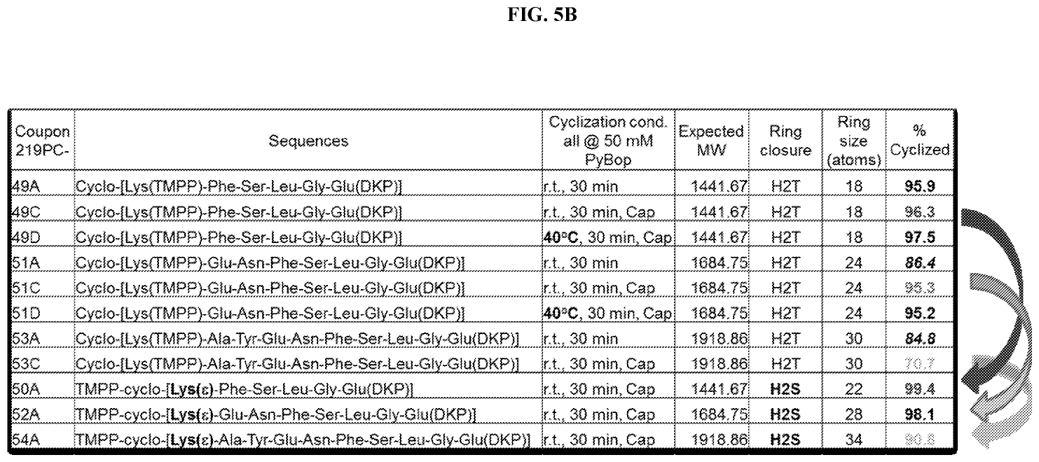

MALDI MS measured yields for constrained peptide arrays using triazole Click chemistry and head-to-sidechain lactam chemistry were determined as per Example 7. Cyclization yields (FIG. 5A) with both chemistries were high for short sequences (B-[AA]ₙ-X, n<5). H2S lactam chemistry gave consistently high yields for longer sequences (n>5). Approximately 90% of peptide features cyclized to >90% in the H2S lactam chemistry. Furthermore, demonstrated cyclization yields for head-to-sidechain are higher than head-to-tail (FIG. 5B).

### Example 19: Determination of Cyclization Efficiency by Fluorescence-based Assay

Following the cyclization reaction, Click and lactam cyclized microarrays were labeled with biotin as described in Example 9 and Example 17, respectively. Slides were extensively washed to remove non-covalently attached biotin prior to the standard operating procedure side chain deprotection reaction described in Example 12. Deprotected slides were incubated with 1.0 nM Streptavidin conjugated to Alexa Fluor™ 555 for 30 min to bind the biotin. Slides were washed, dried and imaged. Individual feature intensities were extracted from the images and analyzed in R using a custom script. BEPANPSKNSTX was selected as an example based on available MALDI data. In the peptide sequence, B and X represent the residues required for cyclization. Boxplots were presented using the 1:50 biotin to cold competitor ratio.

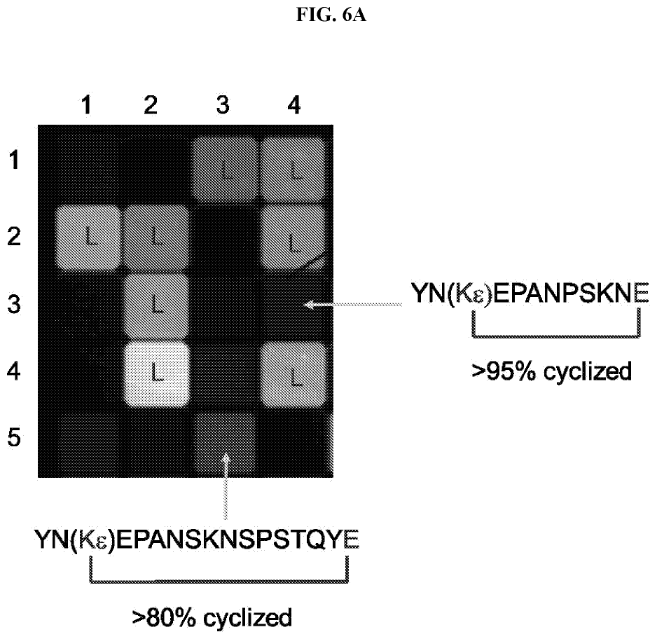

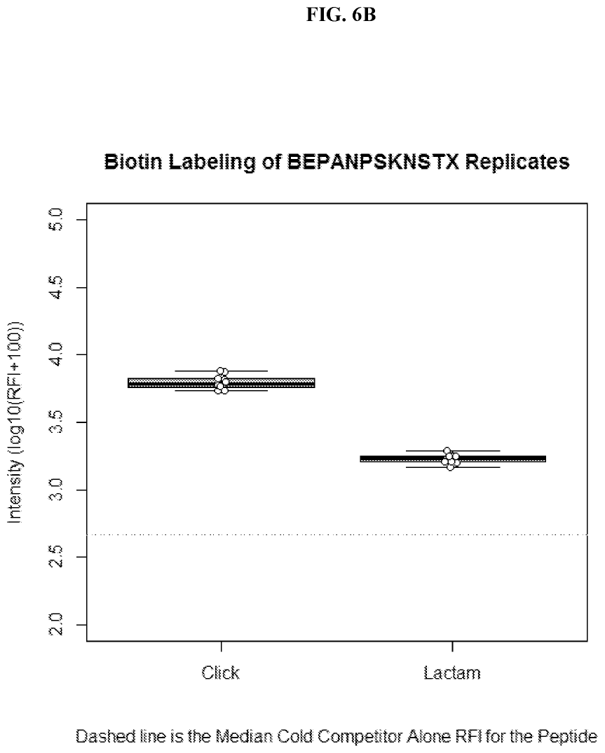

As shown in FIG. 6A, if <50% of peptide molecules have cyclized, strong fluorescence is expected. Peptide features where MALDI analysis indicated >95% cyclization displayed low or very weak fluorescence, while those with <80% cyclization could be observed and determined to not be completely cyclic.

FIG. 6B shows the fluorescence measurements of array peptides that have been labeled with biotin post-cyclization reaction. MALDI analysis indicated the lactam sequence is >98% cyclic while the Click sequence is <50% cyclic. Peptide chains that did not cyclize were labeled with biotin and detected with fluorescent streptavidin. As expected, the nearly quantitatively cyclized lactam peptide has 10-fold lower fluorescence intensity. Points plotted are replicate features within the same array.

### Example 20: Peptide Fluorescence Intensity Based on MALDI Cyclization Efficacy

Data from the slides labeled in Example 19 (FIG. 6) were further analyzed in R using a custom script to relate feature intensity in the biotin labeling assay to the cyclization efficacy calculated from MALDI data. The median replicate feature intensity was calculated for each feature present both on the main array and the MALDI array. Peptides were binned into the following categories: Click chemistry and lactam both greater than 97% cyclic, Click less than 40% cyclic with lactam greater than 97% cyclic, and Click less than 30% cyclic and lactam between 60 and 95% cyclic. Binned intensities were then plotted as a boxplot.

FIG. 7 shows the boxplot of peptide fluorescence intensity based on MALDI cyclization efficacy. Peptide sequences were selected from those present on both the MALDI array and the main library array. Fluorescent intensities are binned based on the MALDI efficacy on the x-axis. Points plotted are median intensity of replicate features, with the same peptides plotted in both the Click and lactam boxes for each section. The fluorescence intensity and MALDI cyclization efficacy generally agree.

### Example 21: Fluorescence Intensity Distributions

Data from the slides labeled in Example 19 (FIG. 6) were further analyzed in R using a custom script to compare the distribution of individual feature intensities. The arrays labeled with 1:50 ratio of biotin to cold competitor and bound by 1.0 nM streptavidin were used to calculate the probability density function (density function in the base R package, default settings). The probability density function was calculated separately for the peptides designed to cyclize and the peptides designed to remain linear.

FIG. 8 shows the fluorescence intensity distributions for both the Click and lactam arrays. Lines plotted represent the probability density function of the fluorescence intensity distribution. Curves labeled linear include the peptide features lacking one of the cyclization residues and are thus solely acyclic, while curves labeled cyclic include the peptide features containing both required cyclization residues. Both linear and cyclic peptides were contained on the same array. The Click array shows two populations corresponding to mostly cyclic, and partially or fully acyclic. The lactam array is mostly cyclic with a small percentage of partially cyclic peptides.

### Example 22: Rituximab Binding to Constrained Peptide Arrays

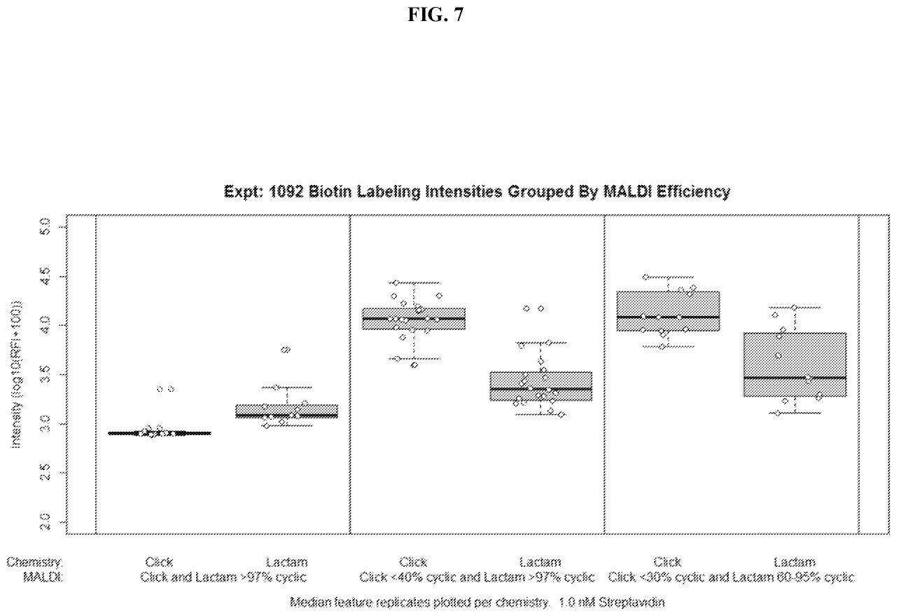

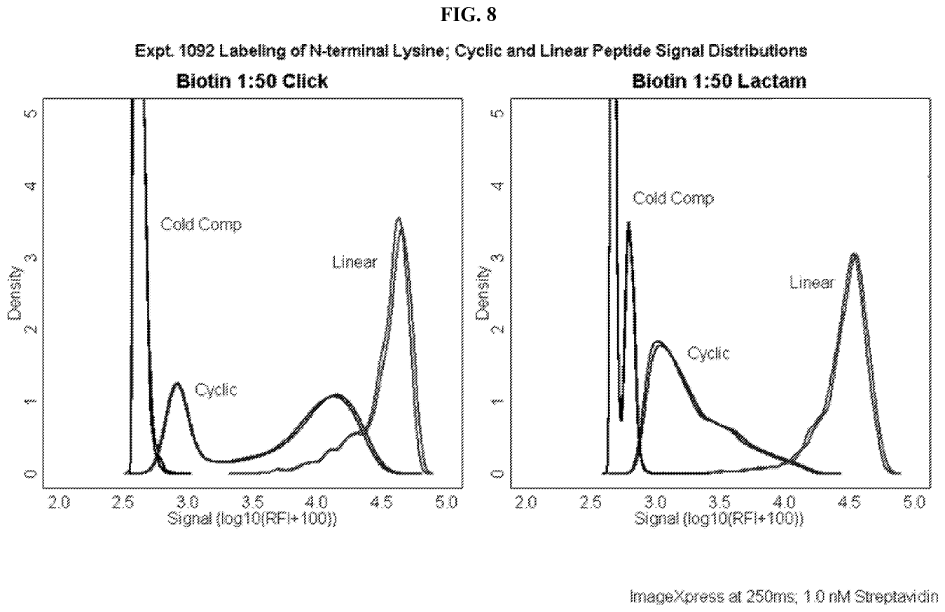

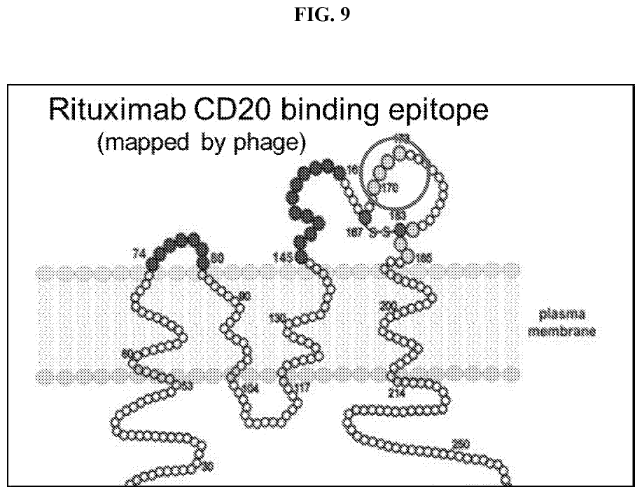

Rituximab is a therapeutic monoclonal antibody (mAb) that targets the extracellular domain of the CD20 receptor (FIG. 9). Rituximab is known to interact with the amino acid loop EPANPSEKNSPSTQY which is bounded on either side by cysteine residues that form a constrained disulfide bridge.

Cyclized and linear microarrays from Click and lactam wafers were probed using a seven point dilution series of Rituximab (Creative Diagnostics, TAB-016) ranging from 24.0 nM to 0.032 nM with threefold steps. Bound Rituximab was detected using an anti-human IgG-Alexa Fluor™ 555 (Thermo-Fisher) at 4.0 nM. All incubation times were for 1 hr and washes between incubation steps were conducted using Phosphate Buffered Saline with 0.05% Tween 20 (PBST). After the secondary detection step, slides were sequentially washed using PBST, distilled water and a 90% isopropanol rinse. Slides were immediately dried by centrifugation following the isopropanol rinse. Imaged arrays were gridded using commercial software to extract intensities from individual features. Data was analyzed using a custom R script. For both chemistries tested, 24.0 nM Rituximab gave the greatest differential between linear and cyclic copies of the same peptide (present on the same array) and was used for example boxplots. Lactam peptide BAEANPSX was amongst the top Rituximab binders and selected for an example plot. In the peptide sequence, B and X represent lactam cyclization residues Lys and Glu respectively. Individual feature replicates for BAEANPSX (cyclic) and BAEANPS (linear) from replicate arrays assayed on two separate slides were used to prepare the boxplot. A p-value comparing the replicate feature intensities was calculated using the anova function in R.

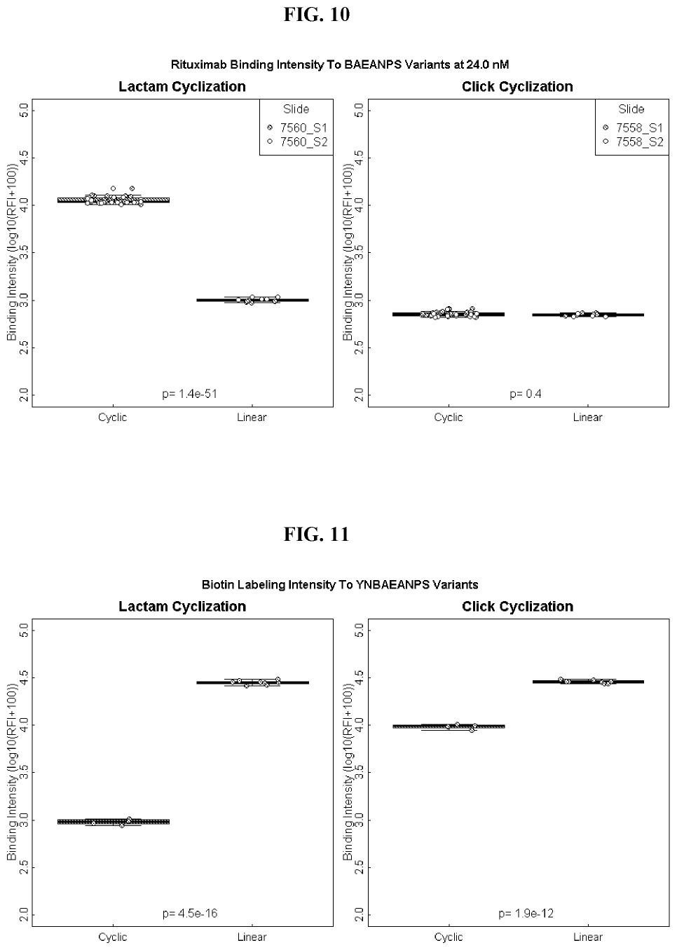

It was found that Rituximab binding to the short peptide BAEANPSX increases upon lactam based cyclization, but not the Click cyclization (FIG. 10). While this could demonstrate an improved binding to the lactam cycled molecules, potentially due to a better 3-dimensional constraint, when the level of cyclization of the peptides was probed, it was found that the Rituximab binding peptide YNBAEANPSX has improved cyclization in lactam chemistry over the Click chemistry (FIG. 11). Thus, the lower amount of cyclization in the Click peptide feature could be the most reasonable explanation for the lower Rituximab binding.

### Example 23: Monoclonal 2117 Binding to Constrained Peptide Arrays

Cyclized and side chain deprotected linear arrays from Click and lactam wafers were probed using a seven point dilution series of 2H7 (Biolegend #302302) ranging from 24.0 nM to 0.032 nM with three-fold steps. Anti-mouse IgG-Alexa Fluor™ 555 (Thermo Fisher) was used to detect bound 2H7. The assay followed the 1 hr incubations and wash steps between incubation steps were conducted using Phosphate Buffered Saline with 0.05% Tween 20 (PBST). After the secondary detection step, slides were sequentially washed using PBST, distilled water and a 90% isopropanol rinse. Slides were immediately dried by centrifugation following the isopropanol rinse. Commercial software was used to extract individual feature intensities from imaged arrays. Data was analyzed using a custom R script.

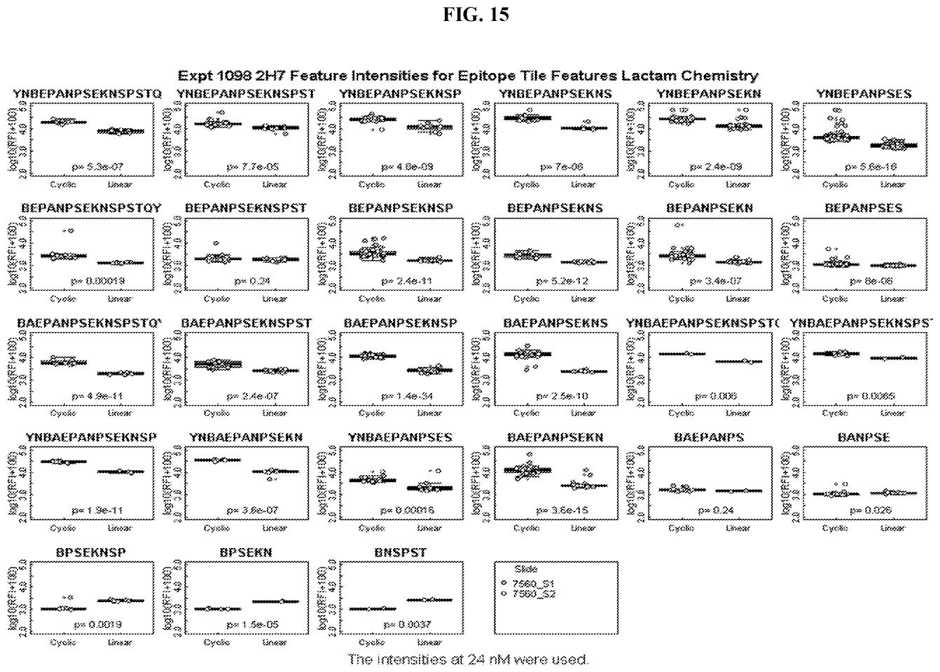

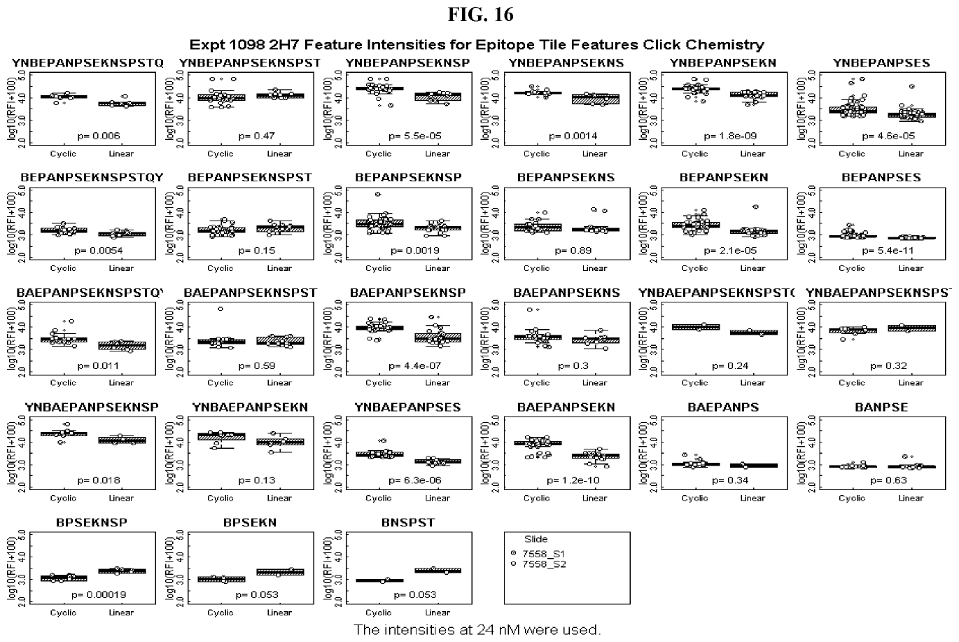

For both the Click and lactam chemistries, the arrays probed with 24.0 nM 2H7 gave the greatest differential between linear and cyclic versions of the same peptide. The peptides selected for inclusion in the boxplot represent those with the key epitope residues (ANPS) for which binding increases upon cyclization, and those without that decrease on cyclization. Individual feature replicates from the cyclized and the linear peptide variants assayed at 24.0 nM on two separate slides were used to prepare the boxplots (FIGS. 15 and 16). Individual replicate features are presented as superimposed points. A p-value comparing the linear and cyclic intensities was calculated for each peptide using the anova function in R.

### Example 24: Solid Phase Synthesis of Cyclo-(KεAEANPSE)-PEG₃-Biotin (HTP115)

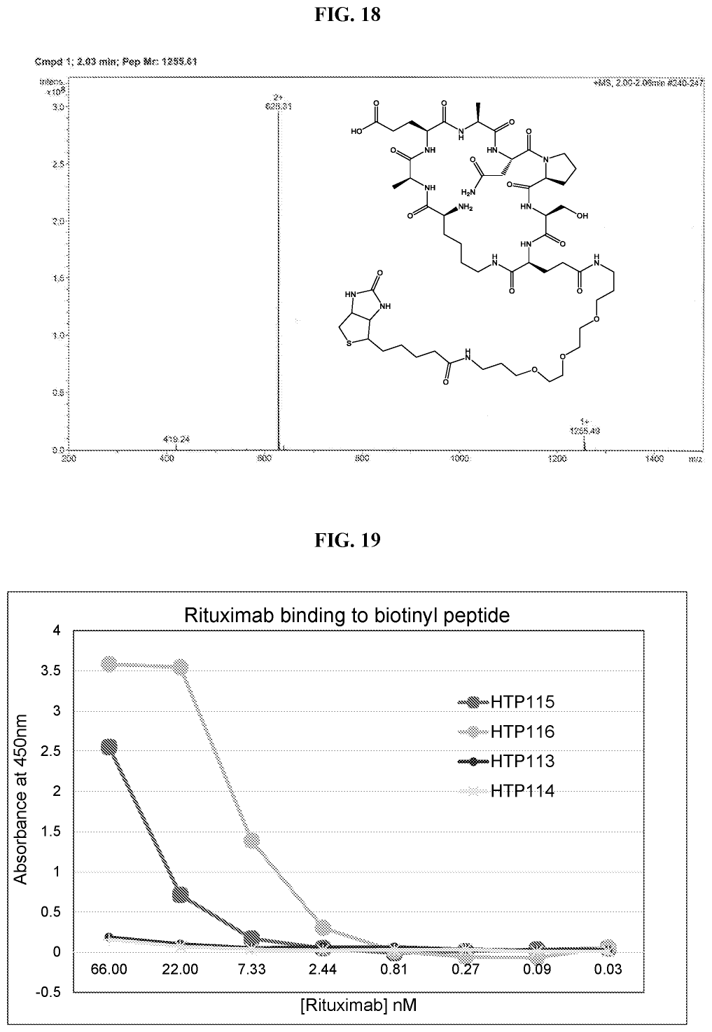

Using standard Fmoc solid phase peptide synthesis methods, Fmoc-Glu(PEG₃-biotin)-OH (CAS #817169-73-6) was coupled to 2-chlorotrityl chloride solid phase resin. Then normal Fmoc amino acid cycles were used to couple Ser(tBu), Pro, Asn(Trt), Ala, Glu(OtBu), Ala, and Boc-Lys(ivDde). The ivDde group was removed by treatment with 2% hydrazine in DMF and the resin washed well. The partially protected peptide was released from the resin by immersion in 20% hexafluoroisopropanol, 1% TFA, 79% DCM for 30 min, the resin was filtered and the filtrate lyophilized. The residue was dissolved in DMF containing 2% DIEA. Cyclization of the Lys side chain amine to the Glu C-terminal acid was accomplished by addition of PyAOP and stirring for 4 hr. The solution was diluted into water and the precipitate collected by centrifugation and dried under vacuum. The solid was dissolved in 95% TFA, 5% TIS for 1 hr, diluted into diethyl ether, centrifuged, and the precipitate dried under vacuum. The crude product was purified by C18 reverse phase HPLC. Fractions containing the product were combined and lyophilized to provide the product. ESMS (FIG. 18) expected monoisotopic MH⁺=1255.6, found=1255.5.

### Example 25: Rituximab Binding to Biotinyl Peptides

To confirm Rituximab binding to the sequences identified in Example 22, the peptides in Table 1 were prepared by Fmoc-based solid phase synthesis. Peptides were biotinylated at either the N-terminal or through the glutamine side chain with a long chain biotin (LCBiot). Biotinylated peptides were bound to streptavidin ELISA plates. Rituximab in PBST was applied to the array in a dilution series ranging from 66.0 to 0.03 nM using three-fold dilution steps. Plates were incubated for 1 hr at 37° C. and washed 3× with PBST. An anti-human IgG-HRP conjugate was applied to the array at 1.0 μg/mL for 1 hr at 37° C. Plates were washed 3× with PBST and TMB substrate applied for 15 min at 37° C. followed by 2N HCl to stop the reaction. Absorbance was read at 450 nm and the median of triplicate wells plotted against mAb concentration in FIG. 19. Rituximab bound constrained peptide HTP115 but not linear version HTP113 or a constrained sequence with LCBiot at the N-terminal (HTP114). Rituximab also binds to HTP116, a biotinylated version of a cyclic disulfide sequence previously reported to bind to Rituximab (Perosa et al. Blood, 2006, 107 (3), 1070-1077).

TABLE 1
Peptides synthesized to confirm Rituximab binding to microarray
peptide sequences

| Peptide | Sequence | Source |
|---|---|---|
| HTP113 | LCBiot-K(Ac)AEANPSQ | Linear form from |
| | amide | Example 22 |
| HTP114 | LCBiot-cyclo[KeAEANPSQ] | Constrained form from |
| | | Example 22 |
| HTP115 | cyclo[KεAEANPSQ(LCBiot)] | Derived from Example |
| | amide | 24 |
| HTP116 | LCBiot-ACPYANPSLC | Reported¹ |
| | amide (cyclic disulfide) | |

¹Perosa et al. Blood, 2006, 107 (3), 1070-1077.

### Example 26: Synthesis of Disulfide Constrained Peptides on SiO₂ by Thallium (III) Oxidation of Cys(MeBzl) Peptides

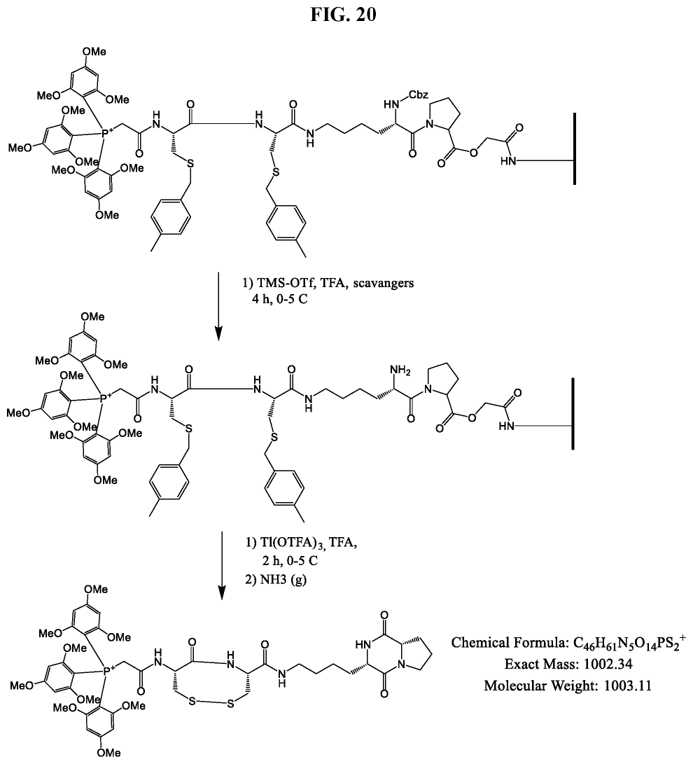

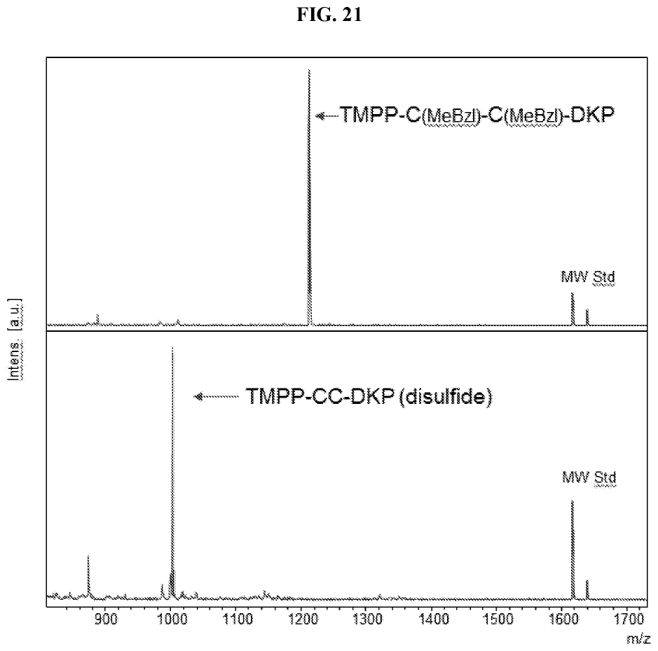

The procedure follows synthetic scheme in FIG. 20. Silicon oxide wafer fragments functionalized with the Cbz protected DKP linker from Example 2 were used. The Cbz-Lys(Boc) was deprotected by treatment with TFA and then Boc-Cys(MeBzl)-OH was coupled to the surface. The cycle was repeated and the N-terminal deprotected with TFA and coupled to TMPP-NHS. The Cbz group was removed as described in Example 12 and the fragments washed twice with 1% TFA/IPA. The fragments were blown dry with N₂ stream. One fragment was then exposed to ammonia and coated with MALDI matrix as described in Example 7. The MALDI mass spectrum of TMPP-Cys(MeBzl)-Cys(MeBzl)-DKP is shown in FIG. 21 (top). The product had the expected m/z peak (calc.=1212.5, found=1212.4). A second fragment was immersed in a solution of 100 mg thallium trifluoroacetate in 10 mL TFA for 2 hrs. The red solution was decanted, the fragment washed twice with 1% TFA/IPA, dried, then exposed to ammonia and coated with MALDI matrix as described in Example 7. The MALDI mass spectrum of the cyclic disulfide TMPP-Cys-Cys-DKP is shown in FIG. 21 (bottom). The product had the expected m/z peak (calc.=1002.4, found=1002.7).

### Example 27: Synthesis of Disulfide Constrained Peptide Microarrays by Air Oxidation of Cysteine Containing Peptides

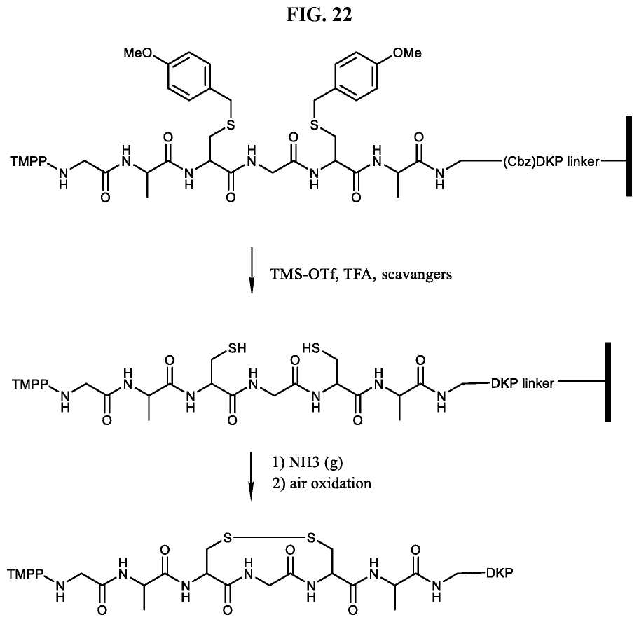

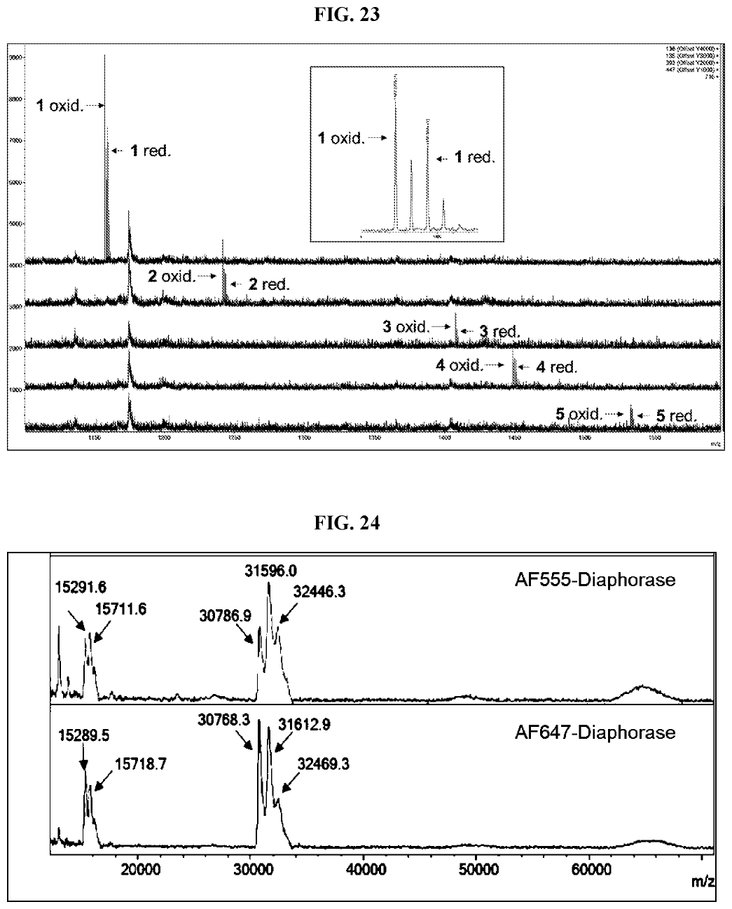

Peptide arrays on SiO₂ surface functionalized with the Cbz protected DKP linker from Example 2 was synthesized as previously described. The arrays were designed so that most of the peptide sequences would include two cysteine residues for eventual oxidation to a cyclic disulfide. Cysteine monomer Boc-Cys(MeOBzl)-OH was used during array synthesis because the methoxybenzyl group is cleanly removed during peptide side chain deprotection as described in Example 12. Following synthesis and side chain deprotection two arrays were treated with ammonia gas to cyclize the DKP and break the covalent linkage to the SiO₂ surface as shown in FIG. 22. The ammonia also neutralizes any residual TFA on the array surface and deprotonates cysteine thiols to thiolates, which are more prone to disulfide formation. The arrays were exposed to ambient air for 30 min or 18 hr then coated with MALDI matrix as described in Example 7. Comparison of the MALDI MS relative peak intensities (RPI) of the oxidized disulfide peptide and reduced peptide (+2.0 amu) enables one to calculate the fraction oxidized as displayed in FIG. 24. After 30 min air exposure peptides were about 25% oxidized. After 18 hrs, peptides were approximately 63-80% oxidized as displayed in Table 2.

TABLE 2

| Spec- | | | | | Frac- | Frac- |
|---|---|---|---|---|---|---|
| tum | TMPP- | Target | RPI | RPI | tion | tion |
| FIG. | Peptide- | M+ | oxi- | re- | Oxi- | Re- |
| 24 | DKP | MW | dized | duced | dized | duced |
| | CC | 1002.4 | 58442 | 14530 | 0.80 | 0.20 |
| | KCCL | 1243.6 | 1735 | 493 | 0.78 | 0.22 |
| | YCCS | 1252.5 | 16264 | 7132 | 0.70 | 0.30 |
| 1 | CPGC | 1156.4 | 49004 | 14956 | 0.77 | 0.23 |
| 2 | CQYC | 1293.4 | 42318 | 16107 | 0.72 | 0.28 |
| | CTHC | 1240.5 | 28404 | 7834 | 0.78 | 0.22 |
| | KCPGCL | 1397.6 | 6622 | 2438 | 0.73 | 0.27 |
| 3 | YCPGCS | 1406.6 | 10084 | 3190 | 0.76 | 0.24 |
| 4 | CQPGYC | 1447.6 | 9424 | 3114 | 0.75 | 0.25 |
| | CTPGHC | 1394.6 | 7658 | 2090 | 0.79 | 0.21 |
| 5 | CTQHYC | 1531.6 | 4144 | 1400 | 0.75 | 0.25 |
| | KCTPGHCL | 1635.7 | 696 | 413 | 0.63 | 0.37 |

### Example 28: Synthesis of Disulfide Constrained Peptide Array by Treatment of Cysteine Containing Peptides with Glutathione Buffer

Peptide microarrays containing Cys(MeOBzl) peptides were subjected to side chain deprotection as described in Example 12. Arrays were washed twice with 1% TFA/IPA then blow dried. The arrays were then immersed for 2 hrs at room temperature in aqueous oxidation buffer (10 mM ammonium bicarbonate, 0.5 mM reduced glutathione (GSH), 2.0 mM oxidized glutathione (GSSG)). The arrays were then immersed for 30 min at room temperature in 10 mM ammonium bicarbonate, washed well with deionized water, washed well with IPA, then blow dried. Disulfide oxidation yields were estimated by fluorescent assay as described in Example 29.

### Example 29: Whole Array Confirmation of Disulfide Formation by Labeling with Alexa Fluor™ 555-Maleimide

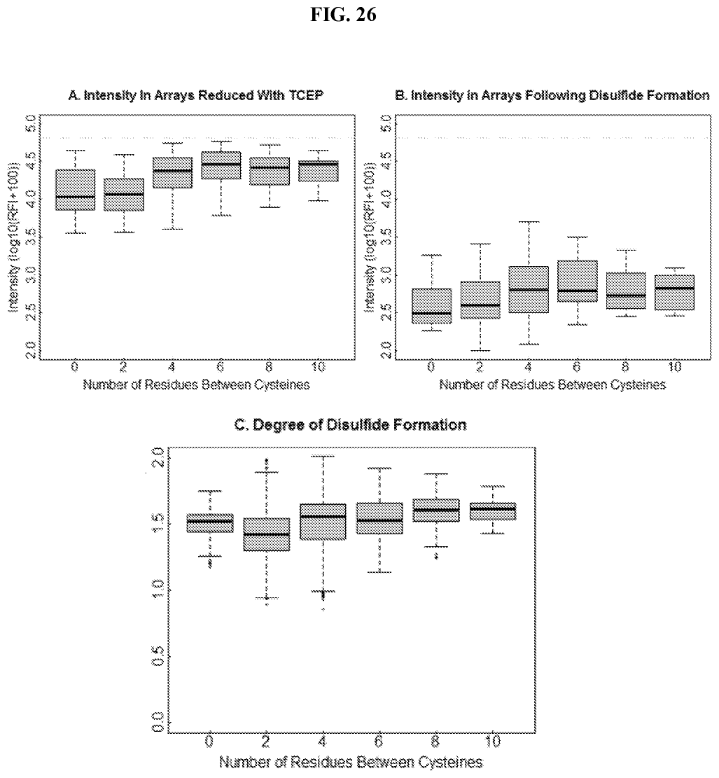

The degree of cyclization by disulfide formation was measured on the array by labeling free cysteines not participating in a disulfide bond. Free thiol groups were labeled using maleimide activated Alexa Fluor™ 555 in the presence of 100 fold excess N-Ethylmaleimide (NEM) as a competitor to attenuate signal. The reaction was conducted in a standard assay cassette in PBS with all reagents at a pH 7.4. Arrays were incubated for 30 min in the presence of 90 uL PBS or 1.0 mM TCEP at room temperature. Without washing, 90 uL Maleimide-Alexa Fluor™ 555 and 100× NEM were added to cassette wells and incubated for 2 hrs at room temperature. Slides were washed 10× in PBS and 4× with distilled water, dried and imaged. The per peptide intensities from the TCEP reduced arrays were used as maximum degree (FIG. 26A) or labeled following the oxidation reaction to form disulfide bonds (FIG. 26B). The delta value (FIG. 26C) was calculated on a per peptide basis as the difference in log 10 transformed intensity values (reduced minus oxidized). Peptide disulfide bonds were successfully formed for each of the loop sizes tested.

### Example 30: Labeling of Diaphorase with Alexa Fluor™ 555-NHS or Alexa Fluor™ 647-NHS

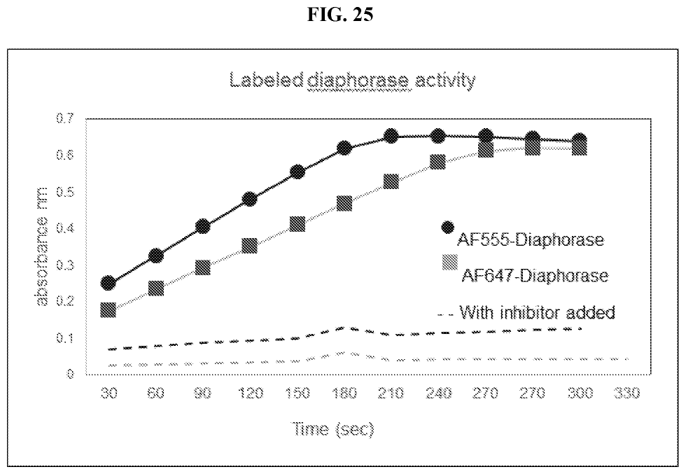

0.40 mg of human diaphorase (NQO1, SigmaAldrich) was dissolved in 0.80 mL of 40 mM NaHCO₃. A 0.1 mg aliquot of Alexa Fluor™ 555-NHS ester (Invitrogen) was dissolved in 200 uL water, quickly transferred to the diaphorase solution, then mixed well. The solution was allowed to react for 90 min at room temperature and was partially purified by passage over a PD MidiTrap G-25 column (GE Healthcare) that was preequilibrated with 0.1 M NH₄OAc. The protein was collected in a 1.5 mL fraction and characterized by MALDI MS. Three major peaks m/z=30780, 31600, 32450 corresponding to 0, 1, and 2 copies of Alexa Fluor™ 555 were observed (FIG. 24, top). Similar results were obtained from a second labeling with Alexa Fluor™ 647-NETS (FIG. 24, bottom). Diaphorase enzymatic activity was confirmed using an NQO1 activity kit (AbCam). As displayed in FIG. 25, both labeled samples retained the activity with the parent unlabeled enzyme. Other proteins including FNR and ferredoxin were labeled by the same method.

### Example 31: Diaphorase Binding to Constrained Peptide Arrays

Diaphorase is NAD(P)H dehydrogenase (UniProtKB: P15559) encoded by the NQO1 gene. Diaphorase has utility as a model enzyme; for which peptide based synthetic affinity agents to diaphorase are required. A 3.3 million feature peptide array was used to identify potential binding partner peptides for diaphorase. Ferredoxin NADP+ Reductase (FNR) and Ferredoxin are included as counter screens for a specific application, while immunoglobulin depleted human serum (Non-Immunoglobulin Serum Components or NISC) was used as a complex mixture to control for generally sticky peptides.

Two wafers from the same synthesis batch were obtained where one wafer was prepared with the lactam chemistry and constrained into a cyclized form. The degree of cyclization was confirmed using the biotinylation assay described in Examples 9, 17, and 19.

Peptide arrays were prepared for protein binding by pretreating the arrays in PBST for 30 min at 57° C. Direct labeled diaphorase (Alexa Fluor™ 555), direct labeled FNR (Alexa Fluor™ 555) and biotinylated ferredoxin were applied to the arrays at 1.0 and 10.0 nM along with direct labeled NISC at 0.001 mg/mL for 1 hr at 37° C. Arrays were washed 3× with PBST and Streptavidin-Alexa Fluor™ 555 was applied to the biotinylated ferredoxin and streptavidin alone arrays. All other arrays received PBST and were incubated for 1 hr at 37° C. Arrays were washed with PBST and p53Ab1-Alexa Fluor™ 647 applied to all arrays to illuminate the edge fiducials for 10 min at 37° C. Following the final incubation arrays were washed in PBST and water, dried and imaged on the ImageXpress (Molecular Dynamics). Triplicate arrays were used for the subsequent analysis except for when rejection of an array was necessary due to the presence of artifacts. Data were analyzed using a custom R script.

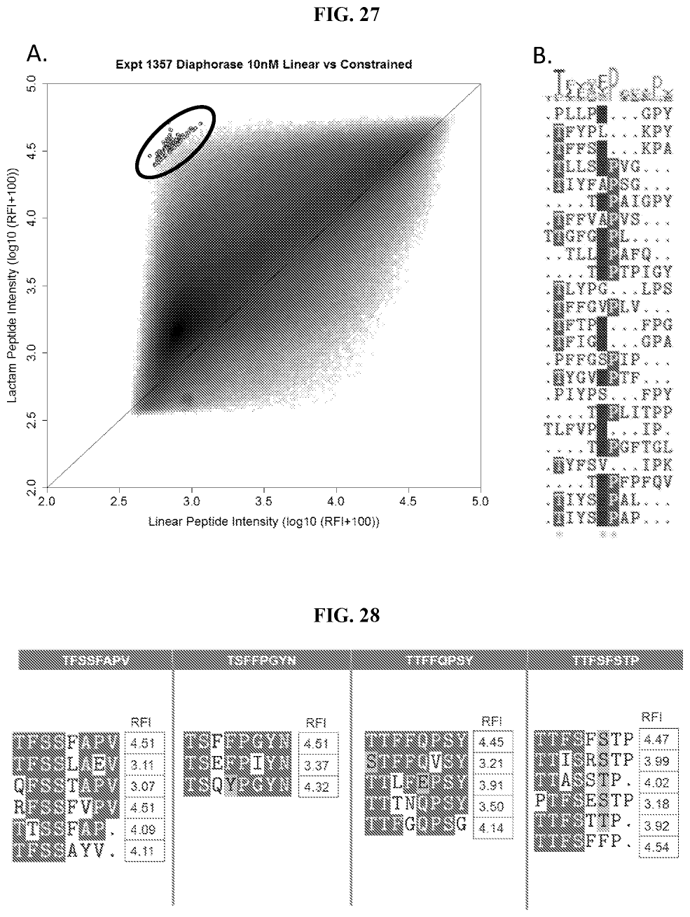

Diaphorase binding to the peptide arrays was notably different depending on whether the peptides were linear or constrained as shown in FIG. 27A. Intensities plotted are the log 10 transformed median intensity from replicate arrays. Coloring of the points represents the density of features at that intensity using a gray-scale map. Light grey represents areas of low peptide numbers while black represents the intensity having the highest number of peptides. The diagonal line is equivalence line. The circled data points represent the lactam constrained sequences that exhibit the greatest enhanced affinity relative to the linear versions. CLUSTAL sequence alignment (FIG. 27B) of the sequences indicated similarities in amino acid composition. Highlighted amino acids are conserved in over 60% of the peptides for that position or are a chemically similar amino acid (ie: F and Y).

The top peptides ranked on the difference in log 10 transformed intensities (LFG_{constrained} minus LFGₗᵢₙₑₐᵣ) and exhibiting nearly 100-fold selectivity over FNR and Ferredoxin are displayed in Table 3. The sequences contain similar amino acid usage. Four peptides having the highest degree of cyclization in the quality control assay were identified and binding intensities compared to other library peptides having one or two amino acid substitutions as shown in FIG. 28. Substitutional analysis provides confidence in the array data by indicating which residues are important for binding. Dark grey boxes highlight indicate a residue conserved in more than 60% of the peptides. Light grey boxes highlight indicate chemically similar amino acids to the conserved residue. RFI is the intensity is the median replicate log 10 intensity for the 10.0 nM Diaphorase.

TABLE 3

| | Diaphorase¹ | | FNR¹ | | Ferredoxin¹ | | NISC² | | Streptavidin³ | | |
|---|---|---|---|---|---|---|---|---|---|---|---|
| | Lactam⁵ | Linear | Lactam | Linear | Lactam | Linear | Lactam | Linear | Lactam | Linear | Closure⁴ |
| TISYPLTP | 4.55 | 2.86 | 2.56 | 2.53 | 2.84 | 2.55 | 3.49 | 2.59 | 2.54 | 2.53 | 0.69 |
| TFSSFAPV | 4.52 | 2.86 | 2.56 | 2.55 | 2.70 | 2.55 | 3.36 | 2.65 | 2.53 | 2.53 | 0.59 |
| TSFFPGYN | 4.51 | 2.86 | 2.62 | 2.54 | 2.71 | 2.54 | 3.47 | 2.60 | 2.52 | 2.53 | 0.61 |
| TFGFNGFP | 4.49 | 2.84 | 2.59 | 2.55 | 2.70 | 2.55 | 3.49 | 2.62 | 2.53 | 2.52 | 0.54 |
| TTFSFSTP | 4.47 | 2.78 | 2.62 | 2.57 | 2.69 | 2.58 | 3.33 | 2.62 | 2.55 | 2.54 | 0.57 |
| TYPIYGPA | 4.46 | 2.77 | 2.59 | 2.53 | 2.80 | 2.55 | 3.43 | 2.59 | 2.54 | 2.52 | 0.59 |
| TTFFQPSY | 4.46 | 2.77 | 2.55 | 2.54 | 2.78 | 2.54 | 3.35 | 2.59 | 2.52 | 2.54 | 0.55 |
| TTLFQTPY | 4.45 | 2.80 | 2.63 | 2.54 | 2.66 | 2.54 | 3.28 | 2.69 | 2.53 | 2.52 | 0.62 |
| TYPGVYPS | 4.40 | 2.75 | 2.59 | 2.54 | 2.69 | 2.54 | 3.16 | 2.58 | 2.53 | 2.51 | 0.53 |

¹10.0 nM protein was applied to the array.
²0.001 mg/mL NISC was applied to the array.
NISC = Non-Immunoglobulin Serum Components.
³Streptavidin-Alexa Fluor ™ 555 was used to detect biotinylated ferredoxin.
⁴Closure is the difference in LFG between the linear and lactam constrained libraries when the lysine involved in cyclization is labeled with biotin.
Biotinylation was done in the quality control assay.
⁵Lactam indicates that the library peptides were cyclized by lactam chemistry

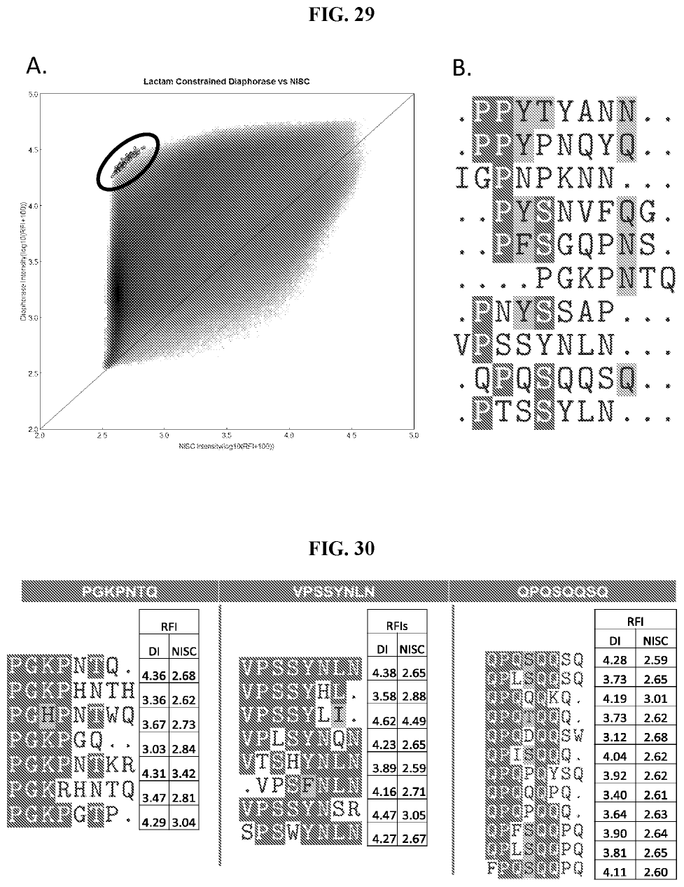

Peptides were also selected based on the difference in log 10 transformed intensities between diaphorase and NISC as the counter screen agent (LFG_{diaphorase} minus LFG_{NISC}). Intensities for 10.0 nM diaphorase and 0.001 mg/mL NISC are shown in FIG. 29A where intensities plotted are the log 10 transformed median intensity from replicate arrays. Coloring of the points represents the density of features at that intensity using a grey colorscale. Light grey represents areas of low peptide numbers while black represents the intensity having the highest number of peptides. The diagonal line is equivalence line. Selectivity vs NISC was not dependent on cyclization as diaphorase also bound the linear forms for some peptides (Table 4). Three peptides having the highest degree of cyclization in the quality control assay were identified and binding intensities compared to other library peptides having one or two amino acid substitutions (FIG. 30) Each CLUSTAL alignment contains the library peptides having a maximum of two substitutions to the parent peptide. Dark grey highlights indicate a residue conserved in more than 60% of the peptides. Light grey highlights indicate chemically similar amino acids to the conserved residue. The amino acid usage is indicated in the sequence logo above the alignment. Substitutional analysis provides confidence in the array data by indicating which residues are important for binding.

TABLE 4

| | Diaphorase¹ | | FNR¹ | | Ferredoxin¹ | | NISC² | | Streptavidin³ | | |
|---|---|---|---|---|---|---|---|---|---|---|---|
| | Lactam⁵ | Linear | Lactam | Linear | Lactam | Linear | Lactam | Linear | Lactam | Linear | Closure⁴ |
| PPYTYANN | 4.35 | 4.00 | 2.54 | 2.53 | 2.56 | 2.55 | 2.66 | 2.63 | 2.53 | 2.52 | 0.79 |
| PPYPNQYQ | 4.39 | 4.04 | 2.55 | 2.56 | 2.58 | 2.58 | 2.69 | 2.69 | 2.53 | 2.53 | 0.75 |
| IGPNPKNN | 4.34 | 4.22 | 2.54 | 2.57 | 2.56 | 2.59 | 2.65 | 2.78 | 2.52 | 2.52 | 0.74 |
| PYSNVFQG | 4.43 | 3.88 | 2.56 | 2.56 | 2.59 | 2.57 | 2.72 | 2.66 | 2.54 | 2.54 | 0.72 |
| PFSGQPNS | 4.38 | 3.70 | 2.58 | 2.58 | 2.59 | 2.59 | 2.67 | 2.63 | 2.57 | 2.56 | 0.71 |
| PGKPNTQ | 4.36 | 4.15 | 2.54 | 2.7 | 2.55 | 2.63 | 2.68 | 2.84 | 2.51 | 2.53 | 0.70 |
| PNYSSAP | 4.31 | 3.91 | 2.56 | 2.54 | 2.56 | 2.55 | 2.63 | 2.61 | 2.52 | 2.52 | 0.69 |
| VPSSYNLN | 4.38 | 3.82 | 2.58 | 2.57 | 2.6 | 2.58 | 2.68 | 2.65 | 2.57 | 2.57 | 0.68 |
| QPQSQQSQ | 4.28 | 3.42 | 2.55 | 2.55 | 2.56 | 2.56 | 2.59 | 2.59 | 2.53 | 2.54 | 0.68 |
| PTSSYLN | 4.32 | 3.75 | 2.53 | 2.53 | 2.56 | 2.55 | 2.63 | 2.61 | 2.52 | 2.53 | 0.67 |

¹10.0 nM protein was applied to the array.
²0.001 mg/mL NISC was applied to the array. Non-Immunoglobulin Serum Components.
³Streptavidin-Alexa Fluor ™ 555 was used to detect biotinylated ferredoxin.
⁴Closure is the difference in LFG between the linear and lactam constrained libraries when the lysine involved in cyclization is labeled with biotin.
Biotinylation was done in the quality control assay.
⁵Lactam indicates that the library peptides were cyclized by lactam chemistry

### Example 32: Confirmation of Lactam Constrained Diaphorase Binding Peptides Identified in Example 31

Peptide sequences are selected from the various sequence alignment families shown in FIG. 28 and FIG. 30 and prepared by solid phase synthesis methods as described in Example 24. Both biotinyl and non-biotinyl versions are used. For example, biotinyl lactam peptides and biotinyl controls are captured on a streptavidin plate and then the plate is washed well. The surface is then incubated with a diaphorase solution under conditions that minimize non-specific binding. Affinity captured diaphorase is detected by adding a colormetric substrate and reading absorbance as with the NQO1 assay described in Example 30.

Alternatively, affinity captured diaphorase can be detected by binding diaphorase specific monoclonal or polyclonal antibodies to captured diaphorase followed by an anti-IgG HRP conjugate.

Alternatively, biotinyl lactam peptides can be captured on a streptavidin chip. Solutions containing different concentrations of diaphorase are flowed over the chip and diaphorase binding detected by surface plasmon resonance on a Biacore instrument. Binding kinetic on-rates and off-rates are calculated from on-progression and off-progression curves, providing equilibrium peptide-diaphorase binding constants.

What is claimed is:

1. A peptide array, comprising at least one cyclic peptide feature which comprises peptides of Formula (I): 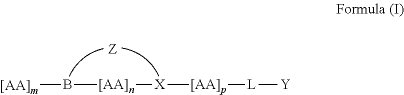
wherein,
each AA is independently a natural or unnatural amino acid residue;
m, n, and p are each independently an integer from 0 to 100;
X is a natural or unnatural amino acid residue;
B is an acid residue; wherein when m is not 0, B is a natural or unnatural amino acid residue;
wherein each AA, B, and X residue is connected to adjacent residues through peptide bonds;
Z is a linker that connects residues X and B;
L is a tether that is optionally present; and
Y is a point of connection connecting the tether to a solid support having a reactive surface.

2. The peptide array of claim 1, wherein each peptide of Formula (I) is independently represented by Formula (Ia) or Formula (Ib): 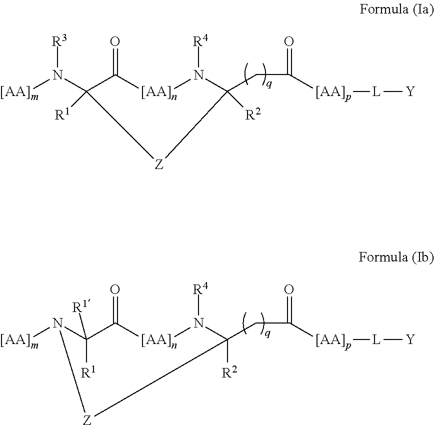
wherein:
q is 0-5;
R¹ and R² are each independently hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl;
R^{1′} is a natural or unnatural amino acid sidechain;
R³ and R⁴ are each independently hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl.

3. The peptide array of either of claim 1 or 2, wherein Z of the at least one cyclic peptide feature is 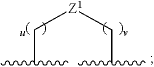
wherein u and v are independently 0-5; and Z¹ is a covalent or non-covalent linkage.

4. The peptide array claim 3, wherein Z¹ is a covalent linkage.

5. The peptide array of claim 4, wherein Z¹ is: 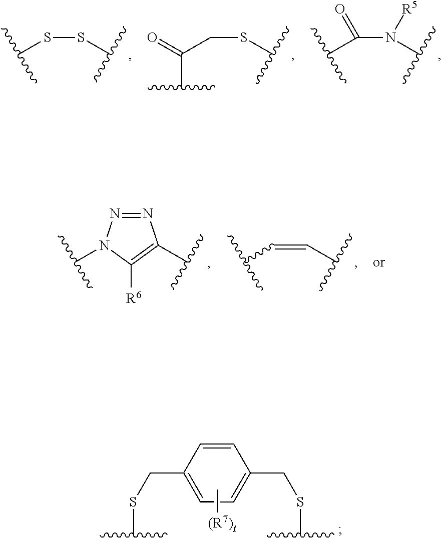
wherein:
R⁵ is hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl;
R⁶ and R⁷ are each independently halogen, hydroxy, C₁-C₆ alkyl, C₁-C₆ alkoxyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl; and
t is 0-4.

6. The peptide array of either of claim 4 or 5, wherein:
R¹ and R² are each independently hydrogen, halogen, or C₁-C₆ alkyl;
R³ and R⁴ are each independently hydrogen or C₁-C₆ alkyl;
R⁵ is hydrogen or C₁-C₆ alkyl; and
R⁶ and R⁷ are each independently halogen, or C₁-C₆ alkyl.

7. The peptide array of any one of claims 4-6, wherein:
R¹ and R² are each hydrogen;
R³ and R⁴ are each hydrogen;
R⁵ is hydrogen; and
t is 0.

8. The peptide array of any one of claims 4-7, wherein Z¹ is

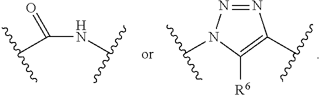

9. The peptide array of any one of claims 4-8, wherein Z¹ is

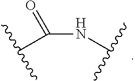 [SMILES: *NC(*)=O]

10. The peptide array of any one of claims 4-8, wherein Z is 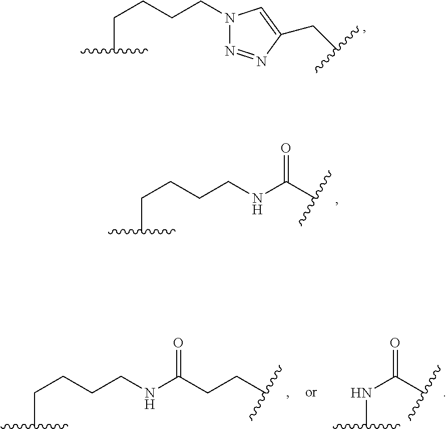 [SMILES: *CCCCn1cc(C*)nn1 ; *CCCCNC(*)=O ; *CCCCNC(=O)CC* ; *NC(*)=O]
[add disulfides]

11. The peptide array of any one of claims 4-7, wherein Z¹ is

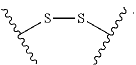 [SMILES: *SS*]

12. The peptide array of claim 11, wherein Z is

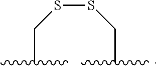 [SMILES: *CSSC*]

13. The peptide array of claim 3, wherein Z¹ is a non-covalent linkage.

14. The peptide array of claim 13, wherein Z¹ is a metal chelate, a salt bridge, or nucleic acid hybridization.

15. The peptide array of any one of claims 1-14, wherein the peptide of Formula (I) of the at least one peptide feature is synthesized from a functionalized peptide of Formula (II): 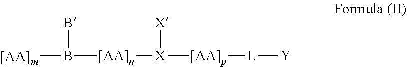
wherein,
each AA is a natural or unnatural amino acid residue;
m, n, and p are each independently an integer from 0 to 100;
X is a natural or unnatural amino acid residue which comprises cyclic precursor X′;
B is an acid residue which comprises cyclic precursor B′;
wherein when m is not 0, B is a natural or unnatural amino acid residue;
wherein each AA, B, and X residue is connected to the neighboring residues through peptide bonds;
L is a tether;
Y is a point of connection connecting the tether to a solid support having a reactive surface; and
wherein X′ and B′ are complementary groups that combine to form Z¹; or
X′ and B′ are groups that each combine with a third group Z² to form Z¹.

16. The peptide array of claim 15, wherein X′ and B′ of Formula (II) are each selected from the group consisting of a thiol, an amine, a carboxylic acid, a haloacetate, a haloalkane, a dihaloalkane, an alkyne, an azide, an alkene, a natural amino acid side chain, an unnatural amino acid side chain, an N-terminal amino group, and a C-terminal carboxyl group.

17. The peptide array of either of claim 15 or 16, wherein X of Formula (II) comprises a sidechain comprising X′.

18. The peptide array of either of claim 15 or 16, wherein X of Formula (II) comprises X′ at the C-terminal head of X.

19. The peptide array of claim 18, wherein X is linked to [AA]ₚ-L- through an amino acid sidechain.

20. The peptide array of any one of claims 15-19, wherein B of Formula (II) comprises a sidechain comprising B′.

21. The peptide array of any one of claims 15-19, wherein B of Formula (II) comprises B′ at the N-terminal tail of B.

22. The peptide array of any one of claims 15-21, wherein B of Formula (II) is a natural or unnatural amino acid.

23. The peptide array of any one of claims 15-21, wherein when m of Formula (II) is 0, B is a natural or unnatural amino acid, or 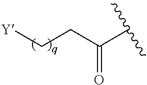
wherein q is 1-4.

24. The peptide array of any one of claims 15-23, wherein B′ of Formula (II) is

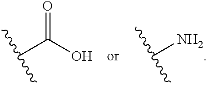

25. The peptide array of any one of claims 15-24, wherein X of Formula (II) is a natural or unnatural amino acid wherein X′ is

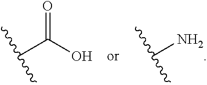

26. The peptide array of any one of claims 15-25, wherein one of X and B of Formula (II) is a suitably modified lysine, ornithine, diaminopropionic, or diaminobutyric acid.

27. The peptide array of any one of claims 15-26, wherein one of X and B of Formula (II) is a suitably modified aspartic or glutamic acid.

28. The peptide array of any one of claims 15-27, wherein X′ of Formula (II) is 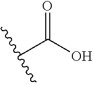 [SMILES: *C(=O)O]
and B′ of the at least one peptide feature is

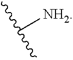 [SMILES: *N]

29. The peptide array of any one of claims 15-23, wherein B′ of Formula (II) is

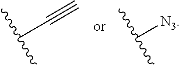

30. The peptide array of any one of claim 15-23 or 29, wherein X′ of Formula (II) is

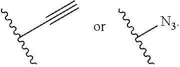

31. The peptide array of any one of claims 15-23, wherein B′ of Formula (II) is

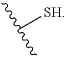 [SMILES: *S]

32. The peptide array of any one of claim 15-23 or 31, wherein X′ of Formula (II) is

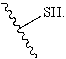 [SMILES: *S]

33. The peptide array of any one of claims 15-32, wherein for a given peptide feature, at least 80% of cyclic precursors X′ and B′ of Formula (II) have combined to form Z¹.

34. The peptide array of claim 33, wherein for a given peptide feature, at least 85%, 90%, 95%, or 97% of cyclic precursors X′ and B′ of Formula (II) have combined to form Z¹.

35. The peptide array of either of claim 33 or 34, wherein the % of cyclic precursors X′ and B′ that have combined to form Z¹ is determined by MALDI analysis.

36. The peptide array of either of claim 33 or 34, wherein the % of cyclic precursors X′ and B′ that have combined to form Z¹ is determined by:
(a) labeling any uncombined X′ or B′ with an affinity handle or a reporter probe;
(b) detecting any uncombined X′ or B′.

37. The peptide array of claim 36, wherein the affinity handle is biotin.

38. The peptide array of claim 37, further comprising a step wherein a fluorescently labeled streptavidin is contacted with the biotin affinity handle.

39. The peptide array of claim 36, wherein the reporter probe is a fluorescent dye comprising a functional group that reacts specifically with uncombined X′ or B′, or both.

40. The peptide array of any one of claims 36-39, wherein any uncombined X′ or B′ in a given peptide feature is detected by fluorescence.

41. The peptide array of any one of claims 1-40, wherein m, n, and p of the at least one cyclic peptide feature are each independently 0-30.

42. The peptide array of any one of claims 1-41, wherein n of the at least one cyclic peptide feature is 0-18.

43. The peptide array of any one of claims 1-42, wherein n of the at least one cyclic peptide feature is 3.

44. The peptide array of any one of claims 1-43, wherein m of the at least one cyclic peptide feature is 0-18.

45. The peptide array of any one of claims 1-44, wherein m of the at least one cyclic peptide feature is 0-2.

46. The peptide array of any one of claims 1-45, further comprising at least one linear peptide feature.

47. The peptide array of claim 46, wherein the at least one linear peptide feature comprises an amino acid sequence substantially the same as that of the at least one cyclic peptide feature.

48. The peptide array of claim 47, wherein the at least one linear peptide feature comprises an amino acid sequence with greater than 80%, 85%, 90%, 95%, or 98% sequence homology with the at least one cyclic peptide feature.

49. The peptide array of claim 48, wherein the at least one linear peptide feature has the same amino acid sequence as the at least one cyclic peptide feature, except that it comprises amino acids at X and B that cannot combine to form linkage Z as in the at least one cyclic peptide feature.

50. The peptide array of any one of claims 1-49, further comprising other cyclic peptide features of Formula (I), wherein each X—Z—B of the other cyclic peptide features is the same as X—Z—B of the at least one cyclic peptide feature.

51. The peptide array of any one of claims 1-49, further comprising other cyclic peptide features of Formula (I), wherein each X—Z—B of the other cyclic peptide features is the same as or different than the X—Z—B of the at least one cyclic peptide feature.

52. The peptide array of any one of claims 1-51, wherein at least one of said peptides comprises a disease-related peptide.

53. The peptide array of any one of claims 1-51, wherein at least one of said peptides comprises a disease-related peptide in a reversed order.

54. The peptide array of any one of claims 1-51, wherein at least one of said peptides comprises a disease-related peptide in a scrambled or randomized order.

55. The peptide array of any one of claims 1-54, wherein at least one of said peptides has greater than 80%, greater than 85%, greater than 90%, greater than 95%, or greater than 98% sequence homology to a disease-related peptide.

56. The peptide array of any one of claims 1-54, wherein at least one of said peptides has greater than 80%, greater than 85%, greater than 90%, greater than 95%, or greater than 98% sequence homology to a disease-related peptide in a reversed order.

57. The peptide array of any one of claims 1-54, wherein at least one of said peptides has greater than 80%, greater than 85%, greater than 90%, greater than 95%, or greater than 98% sequence homology to a disease-related peptide in a scrambled or randomized order.

58. The peptide array of any one of claims 52-57, wherein the disease-related peptide is an epitope, a receptor ligand, a receptor agonist, a receptor antagonist, an enzyme substrate, and enzyme inhibitor, an inhibitor of a protein-protein interaction.

59. The peptide array of any one of claims 1-58, wherein at least one of said peptides is a random peptide sequence.

60. The peptide array of any one of claims 1-59, wherein the peptide features are 5 to 100 amino acids in length.

61. The peptide array of any one of claims 1-60, wherein the peptide features are 5 to 30 amino acids in length.

62. The peptide array of any one of claims 1-61, wherein said array comprises at least about 10,000, 300,000, 1 million, 2 million, or 3 million peptide features per 1 cm².

63. The peptide array of any one of claims 1-62, wherein said solid support is a substrate, bead, polymer, or chromatographic packing material.

64. The peptide array of any one of claims 1-63, wherein said solid support is a Si/SiO₂ wafer.

65. The peptide array of any one of claims 1-64, wherein said peptides are synthesized in situ.

66. A method of synthesizing a peptide array, comprising at least one cyclic peptide feature which comprises peptides of Formula (I): 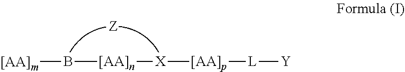
wherein,
each AA is independently a natural or unnatural amino acid residue;
m, n, and p are each independently an integer from 0 to 100;
X is a natural or unnatural amino acid residue;
B is an acid residue; wherein when m is not 0, B is a natural or unnatural amino acid residue;
wherein each AA, B, and X residue is connected to adjacent residues through peptide bonds;
Z is a linker that connects residues X and B;
wherein Z is 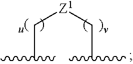
u and v are independently 0-5; and Z′ is a covalent or non-covalent linkage;
L is a tether that is optionally present; and
Y is a point of connection connecting the tether to a solid support having a reactive surface;
the method comprising the step of:
(a) reacting a functionalized peptide of Formula (II): 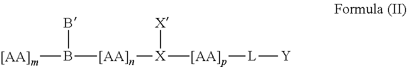
wherein,
wherein X′ and B′ are complementary groups that combine to form Z′; or
X′ and B′ are groups that each combine with a third group Z² to form Z¹;
under conditions that cause linkage Z¹ to form.

67. The method of claim 66, wherein each peptide of Formula (I) is independently represented by Formula (Ia) or Formula (Ib): 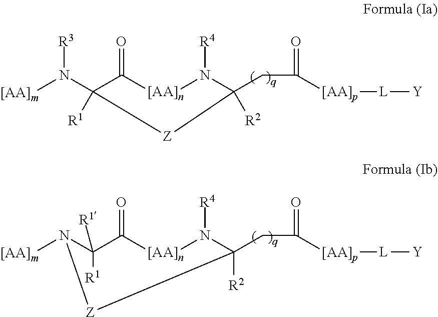
wherein:
q is 0-5;
R¹ and R² are each independently hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl;
R^{1′} is a natural or unnatural amino acid sidechain;
R³ and R⁴ are each independently hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl.

68. The method of either of claim 66 or 67, wherein Z¹ is a covalent linkage.

69. The method of claim 68, wherein Z¹ is: 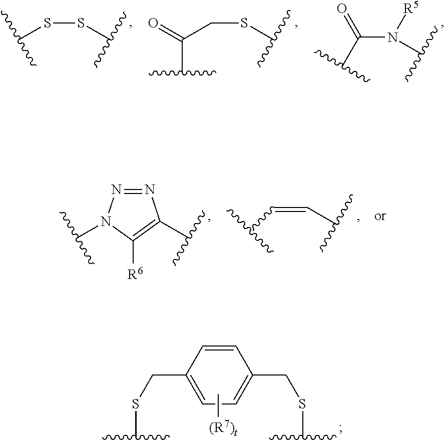
wherein:
R⁵ is hydrogen, C₁-C₆ alkyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl;
R⁶ and R⁷ are each independently halogen, hydroxy, C₁-C₆ alkyl, C₁-C₆ alkoxyl, C₁-C₆ alkenyl, C₁-C₆ alkynyl, cycloalkyl, heterocycloalkyl, aryl, or heteroaryl; wherein alkyl, alkenyl, alkynyl, cycloalkyl, heterocycloalkyl, aryl, and heteroaryl are each unsubstituted or substituted with halogen, hydroxy, alkoxy, or haloalkyl; and
t is 0-4.

70. The method of either of claim 68 or 69, wherein:
R¹ and R² are each independently hydrogen, halogen, or C₁-C₆ alkyl;
R³ and R⁴ are each independently hydrogen or C₁-C₆ alkyl;
R⁵ is hydrogen or C₁-C₆ alkyl; and
R⁶ and R⁷ are each independently halogen, or C₁-C₆ alkyl.

71. The method of any one of claims 68-70, wherein:
R¹ and R² are each hydrogen;
R³ and R⁴ are each hydrogen;
R⁵ is hydrogen; and
t is 0.

72. The method of any one of claims 68-71, wherein Z¹ is

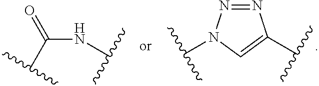 [SMILES: *NC(*)=O ; *c1cn(*)nn1]

73. The method of any one of claims 68-72, wherein Z¹ is

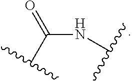 [SMILES: *NC(*)=O]

74. The method of any one of claims 68-72, wherein Z is

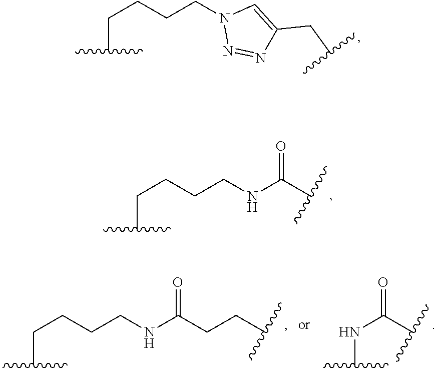 [SMILES: *CCCCn1cc(C*)nn1 ; *CCCCNC(*)=O ; *CCCCNC(=O)CC* ; *NC(*)=O]

75. The method of any one of claims 68-71, wherein Z¹ is

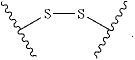 [SMILES: *SS*]

76. The method of claim 75, wherein Z is

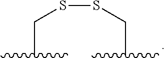 [SMILES: *CSSC*]

77. The method of either of claim 66 or 67, wherein Z¹ is a non-covalent linkage.

78. The method of claim 77, wherein Z¹ is a metal chelate, a salt bridge, or nucleic acid hybridization.

79. The method of any one of claims 66-78, wherein X′ and B′ of Formula (II) are each selected from the group consisting of a thiol, an amine, a carboxylic acid, a haloacetate, a haloalkane, a dihaloalkane, an alkyne, an azide, an alkene, a natural amino acid side chain, an unnatural amino acid side chain, an N-terminal amino group, and a C-terminal carboxyl group.

80. The method of any one of claims 66-79, wherein X of Formula (II) comprises a sidechain comprising X′.

81. The method of any of claims 66-79, wherein X of Formula (II) comprises X′ at the C-terminal head of X.

82. The method of claim 80, wherein X is linked to [AA]ₚ-L- through an amino acid sidechain.

83. The method of any one of claims 66-82, wherein B of Formula (II) comprises a sidechain comprising B′.

84. The method of any one of claims 66-82, wherein B of Formula (II) comprises B′ at the N-terminal tail of B.

85. The method of any one of claims 66-84, wherein B of Formula (II) is a natural or unnatural amino acid.

86. The method of any one of claims 66-84, wherein when m of Formula (II) is 0, B is a natural or unnatural amino acid, or 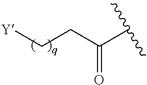
wherein q is 1-4.

87. The method of any one of claims 66-86, wherein B′ of Formula (II) is

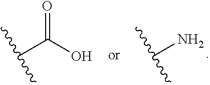

88. The method of any one of claims 66-87, wherein X of Formula (II) is a natural or unnatural amino acid wherein X′ is

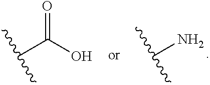

89. The method of any one of claims 66-88, wherein one of X and B of Formula (II) is a suitably modified lysine, ornithine, diaminopropionic, or diaminobutyric acid.

90. The method of any one of claims 66-89, wherein one of X and B of Formula (II) is a suitably modified aspartic or glutamic acid.

91. The method of any one of claim 66-74 or 79-90, wherein:
one of X′ and B′ of the functionalized peptide of Formula (II) is 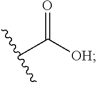 [SMILES: *C(=O)O]
the other of X′ and B′ of the functionalized peptide of Formula (II) is 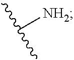 [SMILES: *N]
Z¹ is 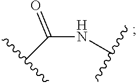 [SMILES: *NC(*)=O]
and
conditions that cause linker Z¹ to form comprise:
i. contacting the peptide of Formula (II) with a suitable amide coupling reagent and a suitable base in a suitable solvent.

92. The method of claim 91, wherein X′ is 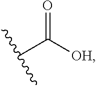 [SMILES: *C(=O)O]
and B′ is

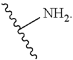 [SMILES: *N]

93. The method of either of claim 91 or 92, wherein the suitable amide coupling reagent is PyBOP.

94. The method of any one of claim 66-72, 74, or 79-86, wherein:
one of X′ and B′ of the functionalized peptide of Formula (II) is 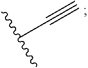 [SMILES: *C#C]
the other of X′ and B′ of the functionalized peptide of Formula (II) is 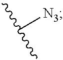 [SMILES: *N=[N+]=[N-]]
Z¹ is 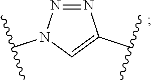 [SMILES: *c1cn(*)nn1]
and
conditions that cause linker Z¹ to form comprise:
i. contacting the peptide of Formula (II) with a solution comprising Cu(I).

95. The method of claim 94, wherein the solution comprising Cu(I) is generated in situ from a Cu(II) salt.

96. The method of claim 95, wherein the Cu(II) salt is CuSO₄.

97. The method of any one of claim 66-71, 75-76, or 79-86, wherein:
both X′ and B′ of the functionalized peptide of Formula (II) is 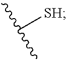 [SMILES: *S]
Z¹ is 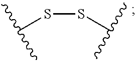 [SMILES: *SS*]
and
conditions that cause linker Z¹ to form comprise:
i. oxidizing the free thiols X′ and B′ to form Z¹.

98. The method of claim 97, wherein the free thiols are oxidized by air oxidation.

99. The method of claim 97, wherein the free thiols are oxidized by a metal catalyst.

100. The method of claim 97, wherein the free thiols are oxidized by an oxidation buffer which comprises oxidized glutathione, cystine, or 5,5′-dithiobis-(2-nitrobenzoic acid) (DTNB).

101. The method of any one of claim 66-71, 75-76, or 79-86, wherein:
both X′ and B′ of the functionalized peptide of Formula (II) is 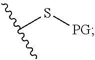
wherein PG is a thiol protecting group;
Z¹ is 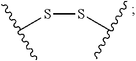 [SMILES: *SS*]
and
conditions that cause linker Z¹ to form comprise:
i. deprotecting X′ and B′ to yield free thiols; and
ii. oxidizing said free thiols to form Z¹.

102. The method of claim 101, wherein the thiol protecting group is an optionally substituted benzyl protecting group.

103. The method of claim 102, wherein the thiol protecting group is

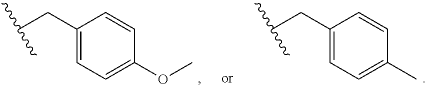

104. The method of any one of claims 101-103, wherein deprotecting the thiol protecting group comprises contacting the functionalized peptide of Formula (II) with a Lewis acid or a metal catalyst.

105. The method of any one of claims 101-103, wherein deprotecting the thiol protecting group comprises contacting the functionalized peptide of Formula (II) with TMS-OTf, TFA, or Tl(OTFA)₃, or a combination thereof.

106. The method of any one of claims 101-105, wherein the free thiols are oxidized by air oxidation.

107. The method of any one of claims 101-105, wherein the free thiols are oxidized by a metal catalyst.

108. The method of any one of claims 101-105, wherein the free thiols are oxidized by an oxidation buffer which comprises oxidized glutathione, cystine, or 5,5′-dithiobis-(2-nitrobenzoic acid) (DTNB).

109. The method of any one of claims 101-108, wherein steps i) and ii) are performed in separate steps.

110. The method of any one of claims 101-108, wherein steps i) and ii) are performed in a single step.

111. The method of any one of claims 66-110, wherein m, n, and p of the at least one cyclic peptide feature are each independently 0-30.

112. The method of any one of claims 66-111, wherein n of the at least one cyclic peptide feature is 0-18.

113. The method of any one of claims 66-112, wherein n of the at least one cyclic peptide feature is 3.

114. The method of any one of claims 66-113, wherein m of the at least one cyclic peptide feature is 0-18.

115. The method of any one of claims 66-114, wherein m of the at least one cyclic peptide feature is 0-2.

116. The method of any one of claims 66-115, wherein the peptide array further comprises at least one linear peptide feature.

117. The method of claim 116, wherein the at least one linear peptide feature comprises an amino acid sequence substantially the same as that of the at least one cyclic peptide feature.

118. The method of claim 117, wherein the at least one linear peptide feature comprises an amino acid sequence with greater than 80%, 85%, 90%, 95%, or 98% sequence homology with the at least one cyclic peptide feature.

119. The method of claim 118, wherein the at least one linear peptide feature has the same amino acid sequence as the at least one cyclic peptide feature, except that it comprises amino acids at X and B that cannot combine to form linkage Z as in the at least one cyclic peptide feature.

120. The method of any one of claims 66-119, wherein the peptide array further comprises other cyclic peptide features of Formula (I), wherein each X—Z—B of the other cyclic peptide features is the same as X—Z—B of the at least one cyclic peptide feature.

121. The method of any one of claims 66-119, wherein the peptide array further comprises other cyclic peptide features of Formula (I), wherein each X—Z—B of the other cyclic peptide features is the same as or different than the X—Z—B of the at least one cyclic peptide feature

122. The method of any one of claims 66-121, wherein at least one of said peptides comprises a disease-related peptide.

123. The method of any one of claims 66-121, wherein at least one of said peptides comprises a disease-related peptide in a reversed order.

124. The method of any one of claims 66-121, wherein at least one of said peptides comprises a disease-related peptide in a scrambled or randomized order.

125. The method of any one of claims 66-124, wherein at least one of said peptides has greater than 80%, greater than 85%, greater than 90%, greater than 95%, or greater than 98% sequence homology to a disease-related peptide.

126. The method of any one of claims 66-124, wherein at least one of said peptides has greater than 80%, greater than 85%, greater than 90%, greater than 95%, or greater than 98% sequence homology to a disease-related peptide in a reversed order.

127. The method of any one of claims 66-124, wherein at least one of said peptides has greater than 80%, greater than 85%, greater than 90%, greater than 95%, or greater than 98% sequence homology to a disease-related peptide in a scrambled or randomized order.

128. The method of any one of claims 122-127, wherein the disease-related peptide is an epitope, a receptor ligand, a receptor agonist, a receptor antagonist, an enzyme substrate, and enzyme inhibitor, an inhibitor of a protein-protein interaction.

129. The method of any one of claims 66-128, wherein at least one of said peptides is a random peptide sequence.

130. The method of any one of claims 66-129, wherein the peptide features are 5 to 100 amino acids in length.

131. The method of any one of claims 66-130, wherein the peptide features are 5 to 30 amino acids in length.

132. The method of any one of claims 66-131, wherein said microarray comprises at least about 10,000, 300,000, 1 million, 2 million, or 3 million peptide features per 1 cm².

133. The method of any one of claims 66-132, wherein said solid support is a substrate, bead, polymer, or chromatographic packing material.

134. The method of any one of claims 66-133, wherein said solid support is a Si/SiO₂ wafer.

135. The method of any one of claims 66-134, wherein said peptides are synthesized in situ.

136. The method of any one of claims 66-135, further comprising the step:
(b) determining which peptide features of a peptide array have successfully cyclized after a cyclization step, and the % to which they have successfully cyclized.

137. The method of claim 136, wherein for a given peptide feature, at least 80% of cyclic precursors X′ and B′ of Formula (II) have combined to form Z¹.

138. The method of either of claim 136 or 137, wherein for a given peptide feature, at least 85%, 90%, 95%, or 97% of cyclic precursors X′ and B′ of Formula (II) have combined to form Z¹.

139. A method for determining which peptide features of a peptide array have successfully cyclized after a cyclization step, and the % to which they have successfully cyclized, using MA LDI mass spectrometry.

140. A method for determining which peptide features of a cyclic peptide microarray have successfully cyclized after a cyclization step, and the % to which they have successfully cyclized, the method comprising:
(a) labeling the uncyclized fraction of the cyclic peptide feature with an affinity handle or a reporter probe;
(b) detecting the uncyclized fraction.

141. The method of claim 140, wherein said labeling occurs through a free amine of the uncyclized fraction.

142. The method of claim 141, wherein the labeling occurs by reacting an NHS ester with the free amine.

143. The method of claim 142, wherein the NHS ester comprises a fluorescent dye.

144. The method of claim 143, wherein the fluorescent dye is an acridine dye, an Alexa Fluor™ dye, a BODIPY dye, a cyanine dye, a fluorone dye, an oxazine dye, a phenanthridine dye, or a rhodamine dye.

145. The method of claim 142, wherein the NHS ester comprises biotin.

146. The method of claim 145, wherein the method further comprises the step of adding a streptavidin-bound fluorescent dye; after step (a) and before step (b).

147. The method of claim 146, wherein the fluorescent dye is an acridine dye, an Alexa Fluor™ dye, a BODIPY dye, a cyanine dye, a fluorone dye, an oxazine dye, a phenanthridine dye, or a rhodamine dye.

148. The method of claim 140, wherein said labeling occurs through an azide of the uncyclized fraction.

149. The method of claim 148, wherein said labeling occurs by reacting a diarylcyclooctyne (DBCO) derivative with the azide.

150. The method of claim 149, wherein the DBCO derivative comprises a fluorescent dye.

151. The method of claim 150, wherein the fluorescent dye is an acridine dye, an Alexa Fluor™ dye, a BODIPY dye, a cyanine dye, a fluorone dye, an oxazine dye, a phenanthridine dye, or a rhodamine dye.

152. A method for characterizing protein binding to peptide targets, the method comprising:
(a) synthesizing a peptide array comprising at least one cyclic peptide feature which comprises peptides of Formula (I): 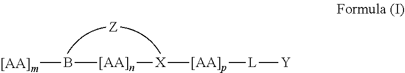
wherein,
each AA is independently a natural or unnatural amino acid residue;
m, n, and p are each independently an integer from 0 to 100;
X is a natural or unnatural amino acid residue;
B is an acid residue; wherein when m is not 0, B is a natural or unnatural amino acid residue;
wherein each AA, B, and X residue is connected to adjacent residues through peptide bonds;
Z is a linker that connects residues X and B;
L is a tether that is optionally present; and
Y is a point of connection connecting the tether to a solid support having a reactive surface;
(b) contacting said peptide array with said protein at one or more concentrations in the presence and absence of a plurality of competitor molecules at one or more concentrations to obtain one or more individual peptide features, wherein the identified one or more individual peptide features exhibit a binding signal measured in the presence of the plurality of competitor molecules at one or more concentrations within a predetermined threshold of the binding signal measured in the absence of the plurality of competitor peptides; and
(c) characterizing binding of the protein against the peptide features on the peptide array.

153. The method of claim 152, wherein the peptide array is synthesized in situ.

154. The method of either of claim 152 or 153, further comprising the steps:
(d) determining which peptide features of said peptide array have successfully cyclized after a cyclization step, and the % to which they have successfully cyclized;
(e) identifying peptide features from the array that have not cyclized to a second predetermined threshold; and
(f) removing peptide features in step (e) from the characterization in step (c).

155. The method of claim 154, wherein the second predetermined threshold of step (e) is at least 50%, at least 60%, at least 70%, at least 75%, at least 80%, at least 85%, at least 90%, or at least 95%.

156. The method of any one of claims 152-155, wherein the predetermined threshold in step (b) is a binding signal in the presence of competitor molecules within at least 20-fold of the binding signal in the absence of competitor peptides.

157. The method of any one of claims 152-156, wherein the predetermined threshold in step (b) is a binding signal in the presence of competitor molecules of at least 5% of the binding signal as compared in the absence of competitor.

158. The method of any one of claims 152-157, wherein said protein is an antibody, a receptor, a receptor ligand, an enzyme, or a protein involved in another peptide-protein interaction.

159. The method of any one of claims 152-158, wherein said competitor molecules comprise peptides.

160. The method of any one of claims 152-159, wherein the competitor molecules comprise a biological sample.

161. The method of claim 160, wherein the biological sample is derived from donor blood products (serum, plasma, blood cells, platelets), tissue samples (donor or tissue culture cell lines), pathogen preparations (intact or lysate), purified antigens (protein or carbohydrate), or purified antibodies (monoclonal or polyclonal, different species, or differently labeled).

162. The method of any one of claims 152-161, wherein the peptide array comprises at least 1000 unique peptide features.

163. The method of any one of claims 152-162, wherein the peptide array comprises at least 10,000 unique peptide features.

164. The method of any one of claims 152-163, wherein the peptide array comprises at least 100,000 unique peptide features.

165. The method of any one of claims 152-164, wherein the peptide array comprises at least 1,000,000 unique peptide features.

166. The method of any one of claims 152-165, wherein the binding signal is measured as an intensity of the signal in the absence and presence of the competitor peptides at one or more concentrations.

167. The method of any one of claims 152-166, wherein an apparent K_{d} is obtained in the presence and absence of the competitor peptides at one or more concentrations.

168. A method for characterizing protein binding to peptide targets, the method comprising:
(a) synthesizing a peptide array comprising at least one cyclic peptide feature which comprises peptides of Formula (I): 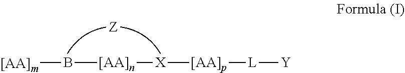
wherein,
each AA is independently a natural or unnatural amino acid residue;
m, n, and p are each independently an integer from 0 to 100;
X is a natural or unnatural amino acid residue;
B is an acid residue; wherein when m is not 0, B is a natural or unnatural amino acid residue;
wherein each AA, B, and X residue is connected to adjacent residues through peptide bonds;
Z is a linker that connects residues X and B;
L is a tether that is optionally present; and
Y is a point of connection connecting the tether to a solid support having a reactive surface;
(b) contacting said peptide array with said protein at one or more concentrations to obtain one or more individual peptide features, wherein the identified one or more individual peptide features exhibit a binding signal measured at one or more concentrations; and
(c) characterizing binding of the protein against the peptide features on the peptide array.

169. The method of claim 168, wherein the peptide array is synthesized in situ.

170. The method of either of claim 168 or 169, further comprising the steps:
(d) determining which peptide features of said peptide array have successfully cyclized after a cyclization step, and the % to which they have successfully cyclized;
(e) identifying peptide features from the array that have not cyclized to a predetermined threshold; and
(f) removing peptide features in step (e) from the characterization in step (c).

171. The method of claim 170, wherein the predetermined threshold of step (e) is at least 50%, at least 60%, at least 70%, at least 75%, at least 80%, at least 85%, at least 90%, or at least 95%.

172. The method of any one of claims 168-171, wherein said protein is an antibody, a receptor, a receptor ligand, an enzyme, or a protein involved in another protein-protein interaction.

173. The method of any one of claims 168-172, wherein the peptide array comprises at least 1000 unique peptide features.

174. The method of any one of claims 168-173, wherein the peptide array comprises at least 10,000 unique peptide features.

175. The method of any one of claims 168-174, wherein the peptide array comprises at least 100,000 unique peptide features.

176. The method of any one of claims 168-175, wherein the peptide array comprises at least 1,000,000 unique peptide features.

177. The method of any one of claims 168-176, wherein the binding signal is measured as an intensity of the signal.
